SEQUENCE LISTING (18 sequences)

SEQ ID NO: 1 - DNA, 72 bp, Artificial Sequence
  Synthetic
ggaaacctgatcatgtagatcgaatggactctaaatccgttcagccgggttagattcccggggtttccgcca

SEQ ID NO: 2 - DNA, 72 bp, Artificial Sequence
  Synthetic
ggaaacctgatcatgtagatcgaatggactttaaatccgttcagccgggttagattcccggggtttccgcca

SEQ ID NO: 3 - DNA, 72 bp, Artificial Sequence
  Synthetic
ggaaacctgatcatgtagatcgaatggacttcaaatccgttcagccgggttagattcccggggtttccgcca

SEQ ID NO: 4 - DNA, 73 bp, Artificial Sequence
  Synthetic
ggaaacctgatcatgtagatcgaatggacttctaaatccgttcagccgggttagattcccggggtttccgcca

SEQ ID NO: 5 - DNA, 747 bp, Artificial Sequence
  Synthetic
atggcatagagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcccgttatccggatcacatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacactaagtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgaactctacaaagagctccatcaccatcaccatcactga

SEQ ID NO: 6 - protein, 248 aa, Artificial Sequence
  Synthetic
MAXSKGEELFTGVVPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHXVYITADKQKNGIKANFKIRHNIEDGSVQLADHYQQNTPIGDGPVLLPDNHYLSTQSALSKDPNEKRDHMVLLEFVTAAGITHGMDELYKELHHHHHH

SEQ ID NO: 7 - DNA, 73 bp, Artificial Sequence
  Synthetic
ggaaacctgatcatgtagatcgaatggacttcctaatccgttcagccgggttagattcccggggtttccgcca

SEQ ID NO: 8 - DNA, 39 bp, Artificial Sequence
  Synthetic
agcgcggccgcgtcgacggtaccctcgagtctggtaaag

SEQ ID NO: 9 - DNA, 40 bp, Artificial Sequence
  Synthetic
attgcggccgcccatggtatatctccttcttatacttaac

SEQ ID NO: 10 - DNA, 36 bp, Artificial Sequence
  Synthetic
tatacatatggcctaggattaaaaattagttgtcgc

SEQ ID NO: 11 - DNA, 33 bp, Artificial Sequence
  Synthetic
cagttccatataggcttaatcgatgttcgggaa

SEQ ID NO: 12 - DNA, 34 bp, Artificial Sequence
  Synthetic
ttcccgaagaatcgattaagcctatatggaactg

SEQ ID NO: 13 - DNA, 45 bp, Artificial Sequence
  Synthetic
gagggtacctcagtgatggtgatggtgatgtttcggttcagtacc

SEQ ID NO: 14 - DNA, 26 bp, Artificial Sequence
  Synthetic
acctgcgataccggtttccacccaag

SEQ ID NO: 15 - DNA, 27 bp, Artificial Sequence
  Synthetic
cttaagttacaggttggtagaaatccc

SEQ ID NO: 16 - DNA, 705 bp, Artificial Sequence
  Synthetic
atggcctaggataaaaaattagttgtcgcgacggataccgccttcgttccgtttgaatttaaacagggcgataaatatgtgggctttgacgttgatctgtgggctgccatcgctaaagagctgaagctggattacgaactgaagccgatggatttcagtgggatcattccggcactgcaaaccaaaaacgtcgatctggcgctggcgggcattaccatcaccgacgagcgtaaaaaagcgatcgatttctctgacggctactacaaaagcggcctgttagtgatggtgaaagctaacaataacgatgtgaaaagcgtgaaagatctcgacgggaaagtggttgcggtgaagagcggcactggctccgttgattacgcgaaagcaaacatcaaaactaaagatctgcgtcagttcccgaacatcgattaagcctatatggaactgggcaccaaccgcgcagacgccgttctgcacgatacgccaaacattctgtacttcatcaaaaccgccggtaacggtcagttcaaagcggtaggcgactctctggaagcgcagcaatacggtattgcgttcccgaaaggtagcgacgagctgcgtgacaaagtcaacggcgcgttgaaaaccctgcgcgagaacggaacttacaacgaaatctacaaaaaatggttcggtactgaaccgaaacatcaccatcaccatcactga

SEQ ID NO: 17 - DNA, 1365 bp, Artificial Sequence
  Synthetic
atggataaaaaaccactaaacactctgatatctgcaaccgggctctggatgtccaggaccggaacaattcataaaataaaacaccacgaagtctctcgaagcaaaatctatattgaaatggcatgcggagaccaccttgttgtaaacaactccaggagcagcaggactgcaagagcgctcaggcaccacaaatacaggaagacctgcaaacgctgcagggtttcggatgaggatctcaataagttcctcacaaaggcaaacgaagaccagacaagcgtaaaagtcaaggtcgtttctgcccctaccagaacgaaaaaggcaatgccaaaatccgttgcgagagccccgaaacctcttgagaatacagaagcggcacaggctcaaccttctggatctaaattttcacctgcgataccggtttccacccaagagtcagtttctgtcccggcatctgtttcaacatcaatatcaagcatttctacaggagcaactgcatccgcactggtaaaagggaatacgaatcccattacatccatgtctgcccctgttcaggcaagtgcccccgcacttacgaagagccagactgacaggcttgaagtcctgttaaacccaaaagatgagatttccctgaattccggcaagcctttcagggagcttgagtccgaattgctctctcgcagaaaaaaagacctgcagcagatctacgcggaagaaagggagaattatctggggaaactcgagcgtgaaattaccaggttctttgtggacaggggttttctggaaataaaatccccgatcctgatccctcttgagtatatcgaaaggatgggcattgataatgataccgaactttcaaaacagatcttcagggttgacaagaacttctgcctgagacccatgatggctccaaacctgctgaactacgcccgcaagcttgacagggccctgcctgatccaataaaaatttttgaaataggcccatgctacagaaaagagtccgacggcaaagaacacctcgaagagtttaccatgctgaacttctgccagatgggatcgggatgtacacgggaaaatcttgaaagcataattacggacttcctgaaccacctgggaattgatttcaagatcgtaggcgattccttcatggtctatggggatacccttgatgtaatgcacggagacctggaactttcctctgcagtagtcggacccataccgcttgaccgggaatggggtattgataaaccctggataggggcaggtttcgggctcgaacgccttctaaaggttaaacacgactttaaaaatatcaagagagctgcaaggtccgagtcttactataacgggatttctaccaacctgtaa

SEQ ID NO: 18 - DNA, 1365 bp, Artificial Sequence
  Synthetic
atggataaaaaaccactaaacactctgatatctgcaaccgggctctggatgtccaggaccggaacaattcataaaataaaacaccacgaagtctctcgaagcaaaatctatattgaaatggcatgcggagaccaccttgttgtaaacaactccaggagcagcaggactgcaagagcgctcaggcaccacaaatacaggaagacctgcaaacgctgcagggtttcggatgaggatctcaataagttcctcacaaaggcaaacgaagaccagacaagcgtaaaagtcaaggtcgtttctgcccctaccagaacgaaaaaggcaatgccaaaatccgttgcgagagccccgaaacctcttgagaatacagaagcggcacaggctcaaccttctggatctaaattttcacctgcgataccggtttccacccaagagtcagtttctgtcccggcatctgtttcaacatcaatatcaagcatttctacaggagcaactgcatccgcactggtaaaagggaatacgaaccccattacatccatgtctgcccctgttcaggcaagtgcccccgcacttacgaagagccagactgacaggcttgaagtcctgttaaacccaaaagatgagatttccctgaattccggcaagcctttcagggagcttgagtccgaattgctctctcgcagaaaaaaagacctgcagcagatctacgcggaagaaagggagaattatctggggaaactcgagcgtgaaattaccaggttctttgtggacaggggttttctggaaataaaatccccgatcctgatccctcttgagtatatcgaaaggatgggcattgataatgataccgaactttcaaaacagatcttcagggttgacaagaacttctgcctgagacccatgcttgctccaaacctttacaactacctgcgcaagcttgacagggccctgcctgatccaataaaaatttttgaaataggcccatgctacagaaaagagtccgacggcaaagaacacctcgaagagtttaccatgctgaacttctgccagatgggatcgggatgcacacgggaaaatcttgaaagcataattacggacttcctgaaccacctgggaattgatttcaagatcgtaggcgattcctgcatggtctatggggatacccttgatgtaatgcacggagacctggaactttcctctgcagtagtcggacccataccgcttgaccgggaatggggtattgataaaccctggataggggcaggtttcgggctcgaacgccttctaaaggttaaacacgactttaaaaatatcaagagagctgcaaggtccgagtcttactataacgggatttctaccaacctgtaa

(19) United States
(12) Patent Application Publication
(10) Pub. No.: US 2013/0078671 A1
(43) Pub. Date: Mar. 28, 2013

(54) INCORPORATION OF TWO DIFFERENT NONCANONICAL AMINO ACIDS INTO A SINGLE PROTEIN

(71) Applicant: Wenshe Liu, College Station, TX (US)

(72) Inventor: Wenshe Liu, College Station, TX (US)

(73) Assignee: THE TEXAS A&M UNIVERSITY SYSTEM, College Station, TX (US)

(21) Appl. No.: 13/430,552

(22) Filed: Mar. 26, 2012

Related U.S. Application Data
(60) Provisional application No. 61/467,728, filed on Mar. 25, 2011.

(51) Int. Cl. C07K 1/107; C12P 21/00

(52) U.S. Cl. CPC C07K 1/107; C12P 21/00
USPC 435/69.1; 530/350; 252/183.11; 435/252.33; 435/252.31; 435/254.21; 435/254.23; 435/358; 435/366; 536/23.1

(57) ABSTRACT

The invention relates to methods, systems, and compositions for the genetic incorporation of a plurality of different noncanonical amino acids into one target protein. The invention provides for multiple, mutually orthogonal aminoacyl-tRNA synthetase/tRNA pairs that suppress two different selector codons engineered into a polynucleotide molecule. By virtue of the suppression of the selector codons, orthogonal aminoacyl-tRNA synthetase/tRNA pairs permit incorporation of their charged noncanonical amino acids into the corresponding positions in the protein. The noncanonical amino acids provide a wide array of functional capabilities. For example, the noncanonical amino acids can provide a reactive pair of moieties that facilitate the study and manipulation of the target protein.

### CROSS-REFERENCE TO RELATED APPLICATION

This application claims the benefit under 35 U.S.C. §1.119(e) of U.S. Application No. 61/467,728, filed Mar. 25, 2011, which is incorporated herein by reference in its entirety.

### STATEMENT OF GOVERNMENT LICENSE RIGHTS

This invention was made with Government support under grant 1R01CA161158 awarded by National Institute of Health. The Government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is: 39063_Seq_Final_{—}2012-03-26.txt. The text file is 12 KB was created on Mar. 26, 2012; and is being submitted via EFS-Web with the filing of the specification.

### BACKGROUND

Methods exist to incorporate unnatural, or noncanonical amino acids (NAAs) site-specifically into proteins in mammalian cells. Chemically aminoacylated suppressor tRNAs have been microinjected or electroporated into CHO cells and neurons, respectively, and used to suppress nonsense amber mutations with a series of unnatural amino acids (Monahan, et al., (2003), “Site-specific incorporation of unnatural amino acids into receptors expressed in Mammalian cells,” Chem Biol 10:573-580). However, the use of the aminoacylated tRNA as a stoichiometric reagent severely limits the amount of protein that can be produced.

Additional approaches have been developed to rationally incorporate noncanonical amino acids in proteins. For example, genetic incorporation of noncanonical amino acids into proteins has been performed at amber UAG codons using bio-orthogonal aminoacyl-tRNA synthetase (aaRS)-amber suppressor tRNA (tRNA_{CUA}) pairs. Evolved Methanococcus jannaschii tyrosyl-tRNA synthetase (MjTyrRS)-MjtRNA(Tyr/CUA) pairs have been used to incorporate a variety of NAAs with different properties into proteins. While the incorporation of these NAAs into proteins has dramatically increased the ability to manipulate protein structure and function, major limitations still exist for the technique. Namely, the technique in general only allows the incorporation of a single NAA into a single protein because the amber codon is the only one available for the incorporation of NAAs. Additionally, the nonsense suppression rate in living cells is low, resulting in a reduced yield of modified protein.

The present disclosure fulfills a need to for a simple, cost effective approach to rationally and predictably incorporate different noncanonical amino acids into a target protein to enhance functional studies of the proteins.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In aspect, the present disclosure provides a method of incorporating at least two different noncanonical amino acids into a target protein, comprising:

(a) providing to a translation system a first polynucleotide encoding a first tRNA mutated to suppress an ochre codon, an opal codon, or a four-base codon, or the first mutant tRNA encoded thereby;

(b) providing to the translation system a second polynucleotide encoding a first aminoacyl tRNA synthetase (aaRS) that can be a mutant or wild-type, or the first aaRS encoded thereby, wherein the first aaRS is capable of charging the first mutant tRNA with a first noncanonical amino acid (NAA);

(c) providing to the translation system a third polynucleotide encoding a second tRNA mutated to suppress an amber codon, or the second mutant tRNA encoded thereby, wherein the second mutant tRNA is orthogonal to the first mutant tRNA and the first aaRS;

(d) providing to the translation system a fourth polynucleotide encoding a second aaRS that can be a mutant or wild-type, or the second aaRS encoded thereby, wherein the second aaRS is capable of charging the second mutant tRNA with a second NAA that is different than the first NAA and wherein the second aaRS is orthogonal to the first aaRS and the first mutant tRNA;

(e) providing to the translation system the first NAA;

(f) providing to the translation system the second NAA;

(g) providing to the translation system a fifth polynucleotide encoding the target protein, wherein the fifth polynucleotide comprises (1) a sequence encoding an ochre codon, an opal codon, or a four-base codon at a first specified position, and (2) a sequence encoding an amber codon at a second specified position; and

(h) allowing translation of the fifth polynucleotide, thereby incorporating into the target protein (1) the first NAA at the first specified position and (2) the second NAA at the second specified position.

It is to be understood that typically the (a)(b) pair is orthogonal to both (i) all endogenous aaRS-tRNA pairs in the translational system, and (ii) to the (c)(d) pair in the same system. Additionally, the (c)(d) pair is orthogonal to both (i) all endogenous aaRS-tRNA pairs in the translational system, and (ii) the (a)(b) pair, in the same system.

In some embodiments, the provisions recited in steps (a)-(g) occur in a host cell. The host cell can be prokaryotic or eukaryotic, including a bacteria cell, a yeast cell, insect cell, or mammalian cell. The host cell can be an Escherichia coli cell, a Bacillus subtilis cell, a Saccharomyces cerevisiae cell, a Pichia pastoris cell, an SF9 cell, a Chinese Hamster Ovary (CHO) cell, or a human cell. Thus, in additional embodiments, both the (a)(b) and (c)(d) pairs of aaRS and cognate tRNAs are orthogonal to all endogenous aaRS-tRNA pairs in the host cell.

In some embodiments, some or all of the provisions recited in steps (a)-(g) occur in a cell-free translation system. In some embodiments, the cell-free translation system is a cell lysate or extract. It will be understood by persons of skill in the art that, in some embodiments, any one or more of the first tRNA, the first aaRS, the second tRNA, and the second aaRS are provided directly to the translation system in their translated, polypeptide forms. Accordingly, in some embodiments, step (a) is performed by contacting the translation system with the first mutant tRNA. In some embodiments, step (a) is performed by contacting the translation system with a solution comprising the first mutant tRNA. In some embodiments, step (b) is performed by contacting the translation system with the first aaRS. In some embodiments, step (b) is performed by contacting the translation system with a solution comprising the first mutant aaRS. In some embodiments, step (c) is performed by contacting the translation system with the second mutant tRNA. In some embodiments, step (c) is performed by contacting the translation system with a solution comprising the second mutant tRNA. In some embodiments, step (d) is performed by contacting the translation system with the second aaRS. In some embodiments, step (d) is performed by contacting the translation system with a solution comprising the second mutant aaRS.

Alternatively, in some embodiments where some or all of the provisions recited in steps (a)-(g) occur in a cell-free translation system, any one or more of the first tRNA, the first aaRS, the second tRNA, and the second aaRS can be provided to the translation system by providing the polynucleotides that encode the one or more of the first tRNA, the first aaRS, the second tRNA, and the second aaRS, respectively, and then allowing the translation thereof based on the encoding provided polynucleotide.

In some embodiments, step (e) is performed by contacting the translation system with a solution comprising the first NAA. In some embodiments, step (f) is performed by contacting the translation system with a solution comprising the second NAA.

In some embodiments, the first NAA and the second NAA are capable of forming a reactive pair. In further embodiments, one of the first NAA and second NAA comprises a donor moiety and the other NAA comprises an acceptor moiety, wherein the donor and acceptor moieties are capable of undergoing Förster resonance energy transfer (FRET). One or both of the donor and acceptor moieties can be incorporated into the cognate NAA before or after the cognate NAA has been incorporated into the target protein.

In some embodiments, the first aaRS is derived from a first organism and the second aaRS is derived from a second organism. In some embodiments, the first aaRS is derived from a Methanococcus mazei aaRS. In some embodiments, the first aaRS is derived from a Methanococcus barkeri aaRS. In some embodiments, the first aaRS is pyrrolysyl-tRNA synthetase (PylRS) or a mutant PylRS. The second aaRS can be derived from a Methanococcus jannascii aaRS. The second aaRS can be a mutant M. jannaschii tyrosyl-tRNA syrithetase (MjTyrRS). Some embodiments can further comprise verifying the incorporation of the first NAA and the second NAA, such as through mass spectrometry.

In another aspect, the present disclosure also provides a translation system comprising:

(a) a first polynucleotide encoding a first tRNA mutated to suppress an ochre codon, an opal codon, or a four-base codon, or the first mutant tRNA encoded thereby;

(b) a second polynucleotide encoding a first aminoacyl tRNA synthetase (aaRS) that can be a mutant or wild-type, or the first aaRS encoded thereby, wherein the first aaRS is capable of charging the first mutant tRNA with a first noncanonical amino acid (NAA);

(c) a third polynucleotide encoding a second tRNA mutated to suppress an amber codon, or the second tRNA encoded thereby, wherein the second mutant tRNA is orthogonal to the first mutant tRNA and the first aaRS;

(d) a fourth polynucleotide encoding second aaRS that can be a mutant or wild-type, or the second aaRS encoded thereby, wherein the second aaRS is capable of charging the second mutant tRNA with a second NAA that is different than the first NAA and wherein the second aaRS is orthogonal to the first aaRS and the first mutant tRNA;

(e) a fifth polynucleotide encoding a target protein, wherein the fifth polynucleotide comprises (1) an ochre codon, an opal codon, or a four-base codon at a first specified position, and (2) an amber codon at a second specified position; and

In some embodiments of the translation system, a host cell comprises components (a)-(e). In alternative embodiments, components (a)-(e) are in a cell-free translation system. In some embodiments, the cell-free translation system is a cell extract or lysate. It will be understood by persons of skill in the art that in some embodiments, any one or more of the first tRNA, the first aaRS, the second tRNA, and the second aaRS are provided directly to the translation system in their translated, polypeptide forms. Accordingly, in some embodiments, part (a) of the translation system is the first mutant tRNA. In some embodiments, part (a) is provided by contacting the translation system with a solution comprising the first mutant tRNA. In some embodiments, part (b) of the translation system is the first aaRS. In some embodiments, part (b) is provided by contacting the translation system with a solution comprising the first mutant aaRS. In some embodiments, part (c) of the translation system is the second mutant tRNA. In some embodiments, part (c) is provided by contacting the translation system with a solution comprising the second mutant tRNA. In some embodiments, part (d) of the translation system is the second aaRS. In some embodiments, part (d) is provided by contacting the translation system with a solution comprising the second mutant aaRS.

Alternatively, in some embodiments where some or all of the components recited in parts (a)-(g) are in a cell-free translation system, any one or more of the first tRNA, the first aaRS, the second tRNA, and the second aaRS are provided to the translation system by providing the polynucleotides that encode the one or more of the first tRNA, the first aaRS, the second tRNA, and the second aaRS, respectively, and then allowing the translation thereof based on the encoding provided polynucleotide.

In some embodiments, the translation system can further comprise a mutant tRNA^{Pyl} (pylT). A translation system according to this aspect can further comprise the first NAA, the second NAA, or both.

In another aspect, the present disclosure provides a mutant pylT comprising a mutation to suppress an ochre codon, an opal codon, or a four-base codon. The pylT can be, e.g., pylTUUA or pylTUCA. The pylT can further comprise, or be charged with, an NAA.

In another aspect, the present disclosure provides a translation system comprising the mutant pylT described herein.

In another aspect, the present disclosure provides one or more vectors for expressing a target protein comprising a noncanonical amino acid, comprising:

(a) a first polynucleotide encoding a first tRNA mutated to suppress an ochre codon, an opal codon, or a four-base codon;

(b) a second polynucleotide encoding a first aminoacyl tRNA synthetase (aaRS) that can be a mutant or wild-type, wherein the first aaRS is capable of charging the first mutant tRNA with a first noncanonical amino acid (NAA); and

(c) a third polynucleotide encoding the target protein, wherein the third polynucleotide comprises (1) an ochre codon, an opal codon, or a four-base codon at a first specified position, and (2) an amber codon at a second specified position.

Embodiments include a single vector comprising components (a)-(c) of this aspect. Additional embodiments include multiple vectors that comprise one or more of the components (a)-(c) of this aspect, wherein multiple different vectors are used to provide all three components (a)-(c) of this aspect.

A vector can be a plasmid. The first tRNA can be pylT. The one or more bevtors can comprise, for example, SEQ ID NO: 2, SEQ ID NO: 3, SEQ ID NO: 4, SEQ ID NO: 5, or SEQ ID NO: 7.

In another aspect, the present disclosure provides an isolated yeast cell or bacterial cell comprising a vector, as described herein. Also provided is an isolated yeast cell or bacterial cell transformed by a vector as provided herein.

In another aspect, the present disclosure also provides a method for incorporating at least two noncanonical amino acids into a target protein, comprising the steps of: (a) providing a first mRNA molecule comprising a first and second stop codon in a reading frame thereof, wherein read-though of the first stop codon results in the incorporation of a first noncanonical amino acid; (b) providing a first orthogonal aaRS, which charges a first mutant suppressor tRNA with a first noncanonical amino acid (NAA) more efficiently than an endogenous aaRS; (c) providing the first suppressor tRNA to incorporate the first noncanonical acid into the polypeptide encoded by the mRNA molecule at the first stop codon; (d) providing a second orthogonal aaRS, which charges a second mutant suppressor tRNA with a second noncanonical amino acid (NAA) more efficiently than an endogenous aaRS; (e) providing the second suppressor tRNA to incorporate the second noncanonical acid into the polypeptide encoded by the mRNA molecule at the second stop codon; wherein the first and second aaRSs are mutually orthogonal, and wherein the first and second suppressor tRNAs are mutually orthogonal, such that at least two different noncanonical amino acids are incorporated into a target protein. In some embodiments, the described components are provided to a cell comprising endogenous protein translation components. In some embodiments, the described components are provided to a cell-free translation system comprising protein translation components.

In another aspect, the present disclosure also provides a method for determining the spatial proximity of two amino acid positions within the tertiary structure of a target polypeptide molecule. The method comprises incorporating a first noncanonical amino acid (NAA) at a first position in the target polypeptide molecule, incorporating a second NAA at a second position in the target polypeptide molecule, wherein the first and second noncanonical amino acids are different and are capable of forming a reactive pair. The method also comprises permitting the polypeptide to assume a tertiary structure and determining if the first NAA and second NAA have formed a reactive pair within the tertiary structure of the polypeptide molecule.

In this aspect, the first NAA and second NAA are incorporated according to the methods, systems and compositions described herein. In some embodiments, one of the first NAA and second NAA comprises a donor moiety, and the other NAA comprises an acceptor moiety, wherein the donor and acceptor moieties are capable of Förster resonance energy transfer (FRET). One or both of the donor and acceptor moieties can be incorporated into the cognate NAA before or after the cognate NAA has been incorporated into the target protein. Upon the assuming a tertiary structure, the polypeptide is assessed for FRET signal, wherein a signal indicates a close proximity of the first NAA and second NAA within the tertiary structure of the polypeptide molecule.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 graphically illustrates the suppression levels of amber (UAG), ochre (UAA), opal (UGA), and four-base UAGA mutations encoding position 149 of GFP_{UV} by their corresponding mutant pylT suppressors, as described in Example 1. The suppression levels are represented by the relative fluorescent emission intensity of expressed full-length GFP_{UV} proteins. The excitation wavelength was 385 nm and the observation wavelength was 505 nm.

FIG. 2A illustrates the structures of noncanonical amino acids (NAAs) 1-4, which were incorporated into the polypeptide of GFP_{UV}, as described in Example 1.

FIG. 2B illustrates the expression level of full length GFP_{UV} with an amber mutation encoding position 1 and an ochre mutation encoding position 149 from cells transformed with pEVOL-AzFRS and pPylRS-pylT-GFP1TAG149TAA at different conditions, as described in Example 1. All NAAs supplemented into media were at 1 mM.

FIG. 2C graphically illustrates the deconvoluted mass spectra generated from ESI-MS analysis of purified GFP_{UV}(1+4), GFP_{UV}(2+4), and GFP_{UV}(3+4), as described in Example 1.

FIG. 3 schematically illustrates the highly efficient one-pot site-specific dual-labeling of the protein in a catalyst free-fashion, as described in Example 2. The genetic incorporation of one azide-containing noncanonical amino acid at an amber mutation site and one different keto-containing noncanonical amino acid at an ochre mutation site, followed by their orthogonal reactions with a cyclooctyne-containing dye and a hydroxylamine-containing dye, respectively.

FIG. 4A illustrates the labeling of QBP(3′+4′) with dye compound 9 and 10, separately and together. The top panel illustrates Coomassie blue stained proteins in a SDS-PAGE gel, whereas the bottom panel illustrates fluorescent imaging of the same gel under irradiation of 365 nm UV light with the image shows real colors captured by a regular camera, as described in Example 2. In color, the bottom panel shows lanes with no band, blue band, green band, and blue/green band, from left to right.

FIG. 4B graphically illustrates the deconvoluted mass spectra generated from ESI-MS analysis of QBP(3′+4′), which has a theoretical molecular weight of 26,028 Da, as described in Example 2.

FIG. 4C graphically illustrates the deconvoluted mass spectra generated from ESI-MS analysis of QBP(3′+4′)-10′, which has a theoretical molecular weight of 26,329 Da, as described in Example 2.

FIG. 4D graphically illustrates the deconvoluted mass spectra generated from ESI-MS analysis of QBP(3′+4′)-9′, which has a theoretical molecular weight of 26,661 Da, as described in Example 2.

FIG. 4E graphically illustrates the deconvoluted mass spectra generated from ESI-MS analysis of QBP(3′+4′)-9′-10′, which has a theoretical molecular weight of 26,962 Da, as described in Example 2.

FIG. 5 graphically illustrates the fluorescent emission spectra of QBP(3′+4′)-9′-10′ at different concentrations of guanidium chloride (GndCl), which indicates decreasing FRET signal as GndCl concentrations increase (leading to increased protein unfolding), as described in Example 2. The excitation wavelength was 430 nm.

FIG. 6 graphically illustrates the dependence of I₄₇₀ₙₘ/I₅₂₀ₙₘ on the concentration of GndCl occurring in the assay illustrated in FIG. 5, and described in Example 2.

### DETAILED DESCRIPTION

A methodology has been developed for the site-specific incorporation of two noncanonical amino acids into proteins. The general concept of orthogonal introduction of noncanonical amino acids into proteins using an aminoacyl-tRNA synthetase (aaRS or RS)-tRNA pair is known. See, e.g., U.S. Publ. Appl. Nos. 2003/0108885 to Schultz et al. and 2008/0254540 to Wang, each incorporated herein by reference in its entirety. However, as described in Example 1, the present disclosure employs two sets of aaRS-tRNA pairs, each orthogonal to each other and each orthogonal to the endogenous aaRS-tRNA pairs, to insert two different noncanonical amino acids into a single protein, wherein each noncanonical amino acid can be inserted at a specific desired position. Furthermore, Example 2 demonstrates that the incorporated NAAs can have donor and acceptor moieties incorporated to perform FRET signaling upon proper folding of the target protein.

As used herein a “noncanonical amino acid” refers to any amino acid other than the following twenty genetically encoded alpha-amino acids: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine. A variety of noncanonical amino acids are known in the art. See, e.g., U.S. Publ. Appl. No. 2003/0108885, incorporated herein by reference in its entirety. For example, a generic structure of an alpha-amino acid is illustrated below in Formula (I):

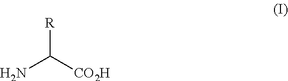

A noncanonical amino acid can be any structure having Formula I wherein the R group is any substituent other than one used in the twenty natural amino acids. Note that noncanonical amino acids can be naturally occurring compounds other than the twenty alpha-amino acids above. Because noncanonical amino acids typically differ from the natural amino acids in side chain only, the noncanonical amino acids form amide bonds with other amino acids, e.g., natural or noncanonical, in the same manner in which they are formed in naturally occurring proteins. However, the noncanonical amino acids have side chain groups that distinguish them from the natural amino acids. For example, R in Formula I optionally comprises an alkyl-, aryl-, acyl-, keto-, azido-, hydroxyl-, hydrazine, cyano-, halo-, hydrazide, alkenyl, alkynl, ether, thiol, seleno-, sulfonyl-, borate, boronate, phospho, phosphono, phosphine, heterocyclic, enone, imine, aldehyde, ester, thioacid, hydroxylamine, amino group, or the like or any combination thereof. Other noncanonical amino acids of interest include, but are not limited to, amino acids comprising a photoactivatable cross-linker, spin-labeled amino acids, fluorescent amino acids, metal binding amino acids, metal-containing amino acids, radioactive amino acids, amino acids with novel functional groups, amino acids that covalently or noncovalently interact with other molecules, photocaged or photoisomerizable amino acids, amino acids comprising biotin or a biotin analogue, glycosylated amino acids such as a sugar substituted serine, other carbohydrate modified amino acids, keto containing amino acids, amino acids comprising polyethylene glycol or polyether, heavy atom substituted amino acids, chemically cleavable or photocleavable amino acids, amino acids with an elongated side chains as compared to natural amino acids, e.g., polyethers or long chain hydrocarbons, e.g., greater than about 5 or greater than about 10 carbons, carbon-linked sugar-containing amino acids, redox-active amino acids, amino thioacid containing amino acids, and amino acids comprising one or more toxic moiety.

In addition to noncanonical amino acids that contain novel side chains, noncanonical amino acids also optionally comprise modified backbone structures, e.g., as illustrated by the structures of Formulas II and III:

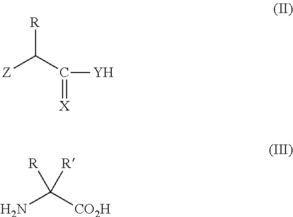

wherein Z typically comprises OH, NH2, SH, NH—R′, or S—R′; X and Y, which can be the same or different, typically comprise S or O, and R and R′, which are optionally the same or different, are typically selected from the same list of constituents for the R group described above for the noncanonical amino acids having Formula I as well as hydrogen. For example, noncanonical amino acids of the invention optionally comprise substitutions in the amino or carboxyl group as illustrated by Formulas II and III. Noncanonical amino acids of this type include, but are not limited to, alpha-hydroxy acids, alpha-thioacids alpha-aminothiocarboxylates, e.g., with side chains corresponding to the common twenty natural amino acids or noncanonical side chains. In addition, substitutions at the alpha-carbon optionally include L, D, or alpha-alpha-disubstituted amino acids such as D-glutamate, D-alanine, D-methyl-O-tyrosine, aminobutyric acid, and the like. Other structural alternatives include cyclic amino acids, such as proline analogues as well as 3, 4, 6, 7, 8, and 9 membered ring proline analogues, beta and gamma amino acids such as substituted beta-alanine and gamma-amino butyric acid.

For example, many noncanonical amino acids are based on natural amino acids, such as tyrosine, glutamine, phenylalanine, and the like. Tyrosine analogs include para-substituted tyrosines, ortho-substituted tyrosines, and meta substituted tyrosines, wherein the substituted tyrosine comprises an acetyl group, a benzoyl group, an amino group, a hydrazine, an hydroxyamine, a thiol group, a carboxy group, an isopropyl group, a methyl group, a C6-C20 straight chain or branched hydrocarbon, a saturated or unsaturated hydrocarbon, an O-methyl group, a polyether group, a nitro group, or the like. In addition, multiply substituted aryl rings are also contemplated. Glutamine analogs of the invention include, but are not limited to, alpha-hydroxy derivatives, beta-substituted derivatives, cyclic derivatives, and amide substituted glutamine derivatives. Example phenylalanine analogs include, but are not limited to, meta-substituted phenylalanines, wherein the substituent comprises a hydroxy group, a methoxy group, a methyl group, an allyl group, an acetyl group, or the like. Specific examples of noncanonical amino acids include, but are not limited to, O-methyl-L-tyrosine, an L-3-(2-naphthyl)alanine, a 3-methyl-phenylalanine, an O-4-allyl-L-tyrosine, a 4-propyl-L-tyrosine, a tri-O-acetyl-GlcNAcbeta-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, and an isopropyl-L-phenylalanine, and the like.

Typically, noncanonical amino acids are selected or designed to provide additional characteristics unavailable in the twenty natural amino acids. For example, noncanonical amino acid are optionally designed or selected to modify the biological properties of a protein, e.g., into which they are incorporated. For example, the following properties are optionally modified by inclusion of a noncanonical amino acid into a protein: toxicity, biodistribution, solubility, stability, e.g., thermal, hydrolytic, oxidative, resistance to enzymatic degradation, and the like, facility of purification and processing, structural properties, spectroscopic properties, chemical or photochemical properties, catalytic activity, redox potential, half-life, ability to react with other molecules, e.g., covalently or noncovalently, and the like.

In some embodiments, a noncanonical amino acid is a derivative of at least one of the 20 natural amino acids, with one or more functional groups not present in natural amino acids, such as keto, bromo-, iodo-, ethynyl-, cyano-, azido-, acetyl, aryl ketone, a photolabile group, a fluorescent group, or a heavy metal. In some embodiments, the noncanonical amino acid is any one in FIGS. 29, 30, and 31 of U.S. Publ. Appl. No. 2003/0108885, incorporated herein by reference in its entirety.

In some embodiments, two noncanonical amino acids form a reactive pair. As used herein, a “reactive pair” of two noncanonical amino acids refers to a first noncanonical amino acid having a first reactive group and a second noncanonical amino acid having a second reactive group, wherein the first reactive group is capable of reacting with the second reactive group. Non-limiting examples of reactive pairs include Förster resonance energy transfer (FRET) fluorophore pairs, which are well-known in the art, and azido-alkynyl pairs that are capable of engaging in click chemistry, as is known in the art.

In some embodiments, the noncanonical amino acids comprise fluorophore moieties that serve as FRET pairs before the noncanonical amino acids are incorporated into the target protein. In other embodiments, the noncanonical amino acids are capable of a post-translational modification in which fluorophore moieties are specifically incorporated into the cognate noncanonical amino acids. In preferred embodiments, the fluorophore moieties are incorporated into the cognate noncanonical amino acids in reactions performed in a “one-pot” dual labeling reaction, thus avoiding the protein aggregation and oxidation problems associated with prolonged, simultaneous labeling procedures, as described in Example 2. Additionally, in preferred embodiments, the fluorophore moieties are incorporated into the cognate noncanonical amino acids in catalyst-free reactions.

The noncanonical amino acid can be 2-amino-8-oxononanoic acid. The noncanonical amino acid can be any of those disclosed in U.S. Publ. Appl. No. 2010/0021963 to Liu et al, incorporated herein by reference in its entirety.

Many of the noncanonical amino acids provided above are commercially available, e.g., from Sigma (USA) or Aldrich (Milwaukee, Wis., USA). Those that are not commercially available are optionally synthesized as provided in various publications or using standard methods known to those of skill in the art. For organic synthesis techniques, see, e.g., Organic Chemistry by Fessendon and Fessendon, (1982, Second Edition, Willard Grant Press, Boston Mass.); Advanced Organic Chemistry by March (Third Edition, 1985, Wiley and Sons, New York); and Advanced Organic Chemistry by Carey and Sundberg (Third Edition, Parts A and B, 1990, Plenum Press, New York). Additional publications describing the synthesis of noncanonical amino acids include, e.g., WO 2002/085923 entitled “In Vivo Incorporation of Noncanonical Amino Acids;” Matsoukas et al., (1995) J. Med. Chem., 38, 4660-4669; King and Kidd (1949) “A New Synthesis of Glutamine and of gamma-Dipeptides of Glutamic Acid from Phthylated Intermediates,” J. Chem. Soc., 3315-3319; Friedman and Chatterrji, (1959) “Synthesis of Derivatives of Glutamine as Model Substrates for Anti-Tumor Agents,” J. Am. Chem. Soc. 81, 3750-3752; Craig et al. (1988) “Absolute Configuration of the Enantiomers of 7-Chloro-4[[4-(diethylamino)-1-methylbutyl]amino]quinoline (Chloroquine),” J. Org. Chem. 53, 1167-1170; Azoulay et al., (1991) “Glutamine analogues as Potential Antimalarials,” Eur. J. Med. Chem. 26, 201-5; Koskinen and Rapoport, (1989) “Synthesis of 4-Substituted Prolines as Conformationally Constrained Amino Acid Analogues,” J. Org. Chem. 54, 1859-1866; Christie and Rapoport, (1985) “Synthesis of Optically Pure

Pipecolates from L-Asparagine. Application to the Total Synthesis of (+)-Apovincamine through Amino Acid Decarbonylation and Iminium Ion Cyclization,” J. Org. Chem. 1989:1859-1866; Barton et al., (1987) “Synthesis of Novel alpha-Amino-Acids and Derivatives Using Radical Chemistry: Synthesis of L- and D-alpha-Amino-Adipic Acids, L-alpha-aminopimelic Acid and Appropriate Unsaturated Derivatives,” Tetrahedron Lett. 43:4297-4308; and, Subasinghe et al., (1992) “Quisqualic acid analogues: synthesis of beta-heterocyclic 2-aminopropanoic acid derivatives and their activity at a novel quisqualate-sensitized site,” J. Med. Chem. 35:4602-7. See also International Publication WO 2004/058946, entitled “PROTEIN ARRAYS,” filed on Dec. 22, 2003.

As used herein, the term “orthogonal” refers to a molecule (e.g., an orthogonal tRNA or an orthogonal aminoacyl-tRNA synthetase) that which associates or reacts with reduced efficiency with a reference molecule (such as compared to a wild-type (e.g., endogenous) or a mutant tRNA or aaRS, as described herein) within a system of interest (e.g., a translational system, e.g., a cell). For example, orthogonality of a mutant aaRS can refer to a reduced efficiency of the aaRS to aminoacylate (“charge”) a reference tRNA (i.e. any endogenous tRNA, or mutant tRNA, other than its cognate tRNA). Similarly, orthogonality of a tRNA results in a reduced efficiency of a tRNA to be aminoacylated (“charged”) by reference aaRS (i.e. any endogenous aaRS, or mutant aaRS, other than its cognate aaRS). In some embodiments, orthogonal refers to the inability or reduced efficiency, e.g., less than 20% efficient, less than 10% efficient, less than 5% efficient, or e.g., less than 1% efficient, of an orthogonal tRNA or orthogonal tRNA synthetase (RS) to function with the reference components in the translation system of interest. See also U.S. Publ. Appl. No. 2003/0108885, incorporated herein by reference in its entirety, in this regard. Thus, as used herein, a first aaRS-tRNA pair can be described as mutually orthogonal with a second first aaRS-tRNA pair, indicating that (1) the first aaRS has a reduced efficiency in aminoacylating the second tRNA, (2) the second aaRS has a reduced efficiency in aminoacylating the first tRNA, (3) the first tRNA has a reduced efficiency in being aminoacylated by the second aaRS, and (4) the second tRNA has a reduced efficiency in being aminoacylated by the first aaRS. The term bio-orthogonal is also used to convey the orthogonality of an aaRS and/or tRNA in reference to the endogenous components of a translation system, whether within a cell or cell lysate.

Any tRNA and any aaRS herein can optionally be considered orthogonal. Methods of determining orthogonality are well-known in the art, and at least one example is provided in this disclosure. In some embodiments, a first mutated tRNA-aaRS pair is orthogonal to both (i) an endogenous tRNA-aaRS (i.e., bio-orthogonal) and (ii) a second mutated tRNA-aaRS pair, and the second mutated tRNA-aaRS pair is also orthogonal to both (i) an endogenous tRNA-aaRS and (ii) the first mutated tRNA-aaRS, in the same system (i.e. mutually orthogonal).

Accordingly, provided herein are orthogonal-tRNAs, orthogonal aminoacyl-tRNA synthetases, and pairs thereof, and pairs that are orthogonal to other pairs. Each of these pairs can be used to incorporate a noncanonical amino acid into growing polypeptide chains and pairs of pairs can be used to incorporate at least two noncanonical amino acids into a single protein.

As used herein, the term “tRNA^{Pyl}” or “pyrrolysyl-tRNA” refer to a tRNA that is specifically charged, or aminoacylated, or capable of being specifically charged, with pyrrolysine by pyrrolysyl-tRNA synthetase, and can include: (1) identical or substantially identical to a naturally occurring tRNA^{Pyl}, (2) derived from a naturally occurring tRNA^{Pyl} by natural or artificial mutagenesis, (3) derived by any process that takes a sequence of a wild-type or mutant tRNA^{Pyl} sequence of (1) or (2) into account, (4) homologous to a wild-type or tRNA^{Pyl}; (5) homologous to any tRNA that is designated as a substrate for a pyrrolysyl-tRNA synthetase, or (6) a conservative variant of any example tRNA that is designated as a substrate for a tRNA^{Pyl} synthetase. The tRNA^{Pyl} can exist charged with an amino acid, or in an uncharged state. It is also to be understood that a tRNA^{Pyl} typically is charged (aminoacylated) by a cognate synthetase with an amino acid other than pyrrolysyl, respectively, e.g., with a noncanonical amino acid. Indeed, it will be appreciated that a tRNA^{Pyl} of the invention is advantageously used to insert essentially any amino acid, whether natural or noncanonical, into a growing polypeptide, during translation, in response to a selector codon (e.g., an ochre codon, an opal codon, or a four-base codon), although typically a noncanonical amino acid is inserted.

As used herein, the term “pyrrolysyl-tRNA synthetase” is an enzyme that preferentially aminoacylates tRNA^{Pyl} with an amino acid in a translation system of interest. The amino acid that the pyrrolysyl-tRNA synthetase loads onto the tRNA^{Pyl} can be any amino acid, whether canonical or noncanonical, and is not limited herein. The synthetase is optionally the same as or homologous to a naturally occurring pyrrolysyl amino acid synthetase. Pyrrolysyl-tRNA synthetases are known in the art. See, e.g., U.S. Publ. Appl. Nos. 2006/0166319 and 2010/0267087, each incorporated herein by reference in its entirety.

As used herein, the terms “tRNA^{Tyr}” or “tyrosyl-tRNA” refers to a tRNA that is a specifically charged, or aminoacylated, or capable of being specifically charged, with tyrrolysine by tyrrolysyl-tRNA synthetase”, where the tRNA is: (1) identical or substantially identical to a naturally occurring tRNA^{Tyr}, (2) derived from a naturally occurring tRNA^{Tyr} by natural or artificial mutagenesis, (3) derived by any process that takes a sequence of a wild-type or mutant tRNA^{Tyr} sequence of (1) or (2) into account, (4) homologous to a wild-type or mutant tRNA^{Tyr}; (5) homologous to any tRNA that is designated as a substrate for a tyrosyl-tRNA synthetase, or (6) a conservative variant of any example tRNA that is designated as a substrate for a tyrosyl-tRNA synthetase. The tRNA^{Tyr} can exist charged with an amino acid, or in an uncharged state. It is also to be understood that an orthogonal tRNA^{Tyr} optionally is charged (aminoacylated) by a cognate synthetase with an amino acid other than tyrosine, respectively, e.g., with a noncanonical amino acid. Indeed, it will be appreciated that an orthogonal tRNA^{Tyr} of the invention is advantageously used to insert essentially any amino acid, whether natural or noncanonical, into a growing polypeptide, during translation, in response to a selector codon (e.g., an amber codon), although typically a noncanonical amino acid is inserted.

As used herein, an orthogonal tyrosyl amino acid synthetase is an enzyme that preferentially aminoacylates the tRNA^{Tyr} with an amino acid in a translation system of interest. The amino acid that the orthogonal tyrosyl amino acid synthetase loads onto the tRNA^{Tyr} can be any amino acid, whether canonical or noncanonical, and is not limited herein. The synthetase is optionally the same as or homologous to a naturally occurring tyrosyl amino acid synthetase.

The term “derived from” refers to a component that is isolated from an organism or isolated and modified, or generated, e.g., chemically synthesized, using information of the component from the organism. tRNAs or aaRSs herein can be derived from any of a variety of organisms (e.g., eukaryotic or non-eukaryotic organisms).

The term “translation system” refers to the components necessary to incorporate a naturally occurring amino acid into a growing polypeptide chain (protein). For example, components can include ribosomes, tRNAs, synthetases, mRNA and the like. The components of the present invention can be added to a translation system, in vivo or in vitro.

In some embodiments, the translation systems are in vitro, and utilize a cell free translation, or synthesis, system. The term “Cell-free synthesis system” refers to the in vitro synthesis of polypeptides in a reaction mix comprising biological extracts and/or defined reagents but without intact, living cells. The reaction mix will comprise a template for production of the macromolecule, e.g. DNA, mRNA, etc.; monomers for the macromolecule to be synthesized, e.g. amino acids, nucleotides, etc.; and cofactors, enzymes and other reagents that are necessary for the synthesis, e.g. ribosomes, uncharged tRNAs, tRNAs charged with unnatural amino acids, polymerases, transcriptional factors, etc.

In some embodiments, the present invention utilizes a cell lysate for in vitro translation of a target protein. For convenience, an organism is used as a source for a lysate, and may be referred to as the source organism or source cell. Source cells may be bacteria, yeast, mammalian or plant cells, or any other type of cell capable of protein synthesis. A lysate comprises components that are capable of translating messenger ribonucleic acid (mRNA) encoding a desired protein, and optionally comprises components that are capable of transcribing DNA encoding a desired protein. Sudh components include, for example, DNA-directed RNA polymerase (RNA polymerase), any transcription activators that are required for initiation of transcription of DNA encoding the desired protein, transfer ribonucleic acids (tRNAs), aminoacyl-tRNA synthetases, 70S ribosomes, N¹⁰-formyltetrahydrofolate, formylmethionine-tRNAf^{Met} synthetase, peptidyl transferase, initiation factors such as IF-I, IF-2, and IF-3, elongation factors such as EF-Tu, EF-Ts, and EF-G, release factors such as RF-1, RF-2, and RF-3, and the like. Lysates are also commercially available from manufacturers such as Promega Corp., Madison, Wis.; Stratagene, La Jolla, Calif.; Amersham, rlington Heights, Ill.; and GIBCO, Grand Island, N.Y.

As will be understood by persons of skill in the art, in some embodiments of a cell-free translation system and methods of use thereof, any one or more of the first tRNA, the first aaRS, the second tRNA, and the second aaRS exist in, or are provided directly to, the translation system in their translated, polypeptide forms. Alternatively, in some embodiments, any one or more of the first tRNA, the first aaRS, the second tRNA, and the second aaRS exist in, or are provided to, the translation system in the encoding polynucleotide forms. In these embodiments, the translation system is permitted to generate the polypeptide forms using the encoding polynucleotide forms as template.

A “host cell” refers to any cell of an organism that is selected, modified, transformed, grown or used or manipulated in any way for the production of a substance by the cell. For example, a host cell can be one that is manipulated to express a particular gene, a DNA or RNA sequence, a protein, or an enzyme. Non-limiting examples of host cells include bacterial cells and yeast cells.

As used herein in reference to orthogonal translation systems, an orthogonal-RS “preferentially aminoacylates” a cognate orthogonal-tRNA when:

(i) the orthogonal-RS charges the orthogonal-tRNA with a noncanonical amino acid more efficiently than it charges any endogenous tRNA in an expression system. That is, when the orthogonal-tRNA and any given endogenous tRNA are present in a translation system in approximately equal molar ratios, the orthogonal-RS will charge the orthogonal-tRNA more frequently than it will charge the endogenous tRNA. Preferably, the relative ratio of orthogonal-tRNA charged by the orthogonal-RS to endogenous tRNA charged by the orthogonal-RS is high, preferably resulting in the orthogonal-RS charging the orthogonal-tRNA exclusively, or nearly exclusively, when the orthogonal-tRNA and endogenous tRNA are present in equal molar concentrations in the translation system. The relative ratio between orthogonal-tRNA and endogenous tRNA that is charged by the orthogonal-RS, when the orthogonal-tRNA and orthogonal-RS are present at equal molar concentrations, is greater than 1:1, preferably at least about 1:1, more preferably 5:1, still more preferably 10:1, yet more preferably 20:1, still more preferably 50:1, yet more preferably 75:1, still more preferably 95:1, 98:1, 99:1, 100:1, 500:1, 1,000:1, 5,000:1 or higher; and

(ii) the orthogonal-RS charges the orthogonal-tRNA with a noncanonical amino acid more efficiently than it charges any second, mutant tRNA in an expression system. That is, when the orthogonal-tRNA and any given second, mutant tRNA are present in a translation system in approximately equal molar ratios, the orthogonal-RS will charge the orthogonal-tRNA more frequently than it will charge the second, mutant tRNA. Preferably, the relative ratio of orthogonal-tRNA charged by the orthogonal-RS to second, mutant tRNA charged by the orthogonal-RS is high, preferably resulting in the orthogonal-RS charging the orthogonal-tRNA exclusively, or nearly exclusively, when the orthogonal-tRNA and second, mutant tRNA are present in equal molar concentrations in the translation system. The relative ratio between orthogonal-tRNA and second, mutant tRNA that is charged by the orthogonal-RS, when the orthogonal-tRNA and orthogonal-RS are present at equal molar concentrations, is greater than 1:1, preferably at least about 2:1, more preferably 5:1, still more preferably 10:1, yet more preferably 20:1, still more preferably 50:1, yet more preferably 75:1, still more preferably 95:1, 98:1, 99:1, 100:1, 500:1, 1,000:1, 5,000:1 or higher.

The term “cognate” refers to components that function together, or have some aspect of specificity for each other, e.g., an orthogonal tRNA and an orthogonal aminoacyl-tRNA synthetase (aaRS or RS). The components can also be referred to as being complementary.

The term “selector codon” refers to codons recognized by the orthogonal-tRNA in the translation process and not recognized by an endogenous tRNA. Such codons are known in the art. The orthogonal-tRNA anticodon loop recognizes the selector codon on the mRNA and incorporates its amino acid, e.g., a noncanonical amino acid, at this site in the polypeptide. Selector codons can include, e.g., nonsense codons, such as, stop codons, e.g., amber, ochre, and opal codons; four or more base codons; rare codons; codons derived from natural or noncanonical base pairs or the like. In some aspects, a selector codon of the invention is a suppressor codon, e.g., a stop codon (e.g., an amber, ochre or opal codon), or a suppressor four-base codon. The selector codon may also be referred to as a blank codon, indicating the lack of an endogenous tRNA that can recognize and/or bind the codon with sufficient affinity and/or avidity to incorporate the cognate amino acid in the polypeptide before termination of the translation process.

The term “four-base codon” refers to four-base codons recognized by a mutated tRNA in the translation process and not recognized by an endogenous tRNA. Such codons are known in the art. Examples of four base codons include AGGA, CUAG, UAGA, and CCCU. Four-base codons can be suppressor codons.

Polynucleotides are also a feature of the invention. A polynucleotide disclosed herein includes an artificial (e.g., man-made, and not naturally occurring) polynucleotide comprising a nucleotide sequence encoding a polypeptide, such as those set forth in sequence listings herein, or is complementary to that polynucleotide sequence. A polynucleotide can also include a nucleic acid that hybridizes to a polynucleotide, under highly stringent conditions, over substantially the entire length of the nucleic acid. A polynucleotide of the invention also includes a polynucleotide that is, e.g., at least 75%, at least 80%, at least 90%, at least 95%, at least 98% or more identical to that of a naturally occurring tRNA or corresponding coding nucleic acid (but a polynucleotide of the invention is other than a naturally occurring tRNA or corresponding coding nucleic acid), where the tRNA recognizes a selector codon, e.g., a four-base codon. Artificial polynucleotides that are, e.g., at least 80%, at least 90%, at least 95%, at least 98% or more identical to any of the above or a polynucleotide comprising a conservative variation of any the above, are also included in polynucleotides of the invention.

In some embodiments, an orthogonal-tRNA comprises or is encoded by a polynucleotide sequence as set forth in the sequence listings (e.g., SEQ ID NO: 2, SEQ ID NO: 3, SEQ ID NO: 4, or SEQ ID NO: 7) or examples herein, or a complementary polynucleotide sequence thereof. In some embodiments, an orthogonal-RS or a portion thereof is encoded by a polynucleotide sequence encoding an amino acid as set forth in the examples herein, or a complementary polynucleotide sequence thereof.

Vectors comprising a polynucleotide of the invention are also a feature of the invention. For example, a vector of the invention can include a plasmid, a cosmid, a phage, a virus, an expression vector, or the like. A cell comprising a vector of the invention is also a feature of the invention.

Methods of producing components of an orthogonal-tRNA/orthogonal-RS pair are also features of the invention. Components produced by these methods are also a feature of the invention. For example, methods of producing at least one tRNA that is orthogonal to a cell (orthogonal-tRNA) include generating a library of mutant tRNAs; mutating an anticodon loop of each member of the library of mutant tRNAs to allow recognition of a selector codon, thereby providing a library of potential orthogonal-tRNAs, and subjecting to negative selection a first population of cells of a first species, where the cells comprise a member of the library of potential orthogonal-tRNAs. The negative selection eliminates cells that comprise a member of the library of potential orthogonal-tRNAs that is aminoacylated by an aminoacyl-tRNA synthetase (RS) that is endogenous to the cell. This provides a pool of tRNAs that are orthogonal to the cell of the first species, thereby providing at least one orthogonal-tRNA. An orthogonal-tRNA produced by the methods of the invention is also provided.

In some embodiments, the methods further comprise subjecting to positive selection a second population of cells of the first species, where the cells comprise a member of the pool of tRNAs that are orthogonal to the cell of the first species, a cognate aminoacyl-tRNA synthetase, and a positive selection marker. Using the positive selection, cells are selected or screened for those cells that comprise a member of the pool of tRNAs that is aminoacylated by the cognate aminoacyl-tRNA synthetase and that shows a desired response in the presence of the positive selection marker, thereby providing an orthogonal-tRNA. In some embodiments, the second population of cells comprises cells that were not eliminated by the negative selection.

Methods for identifying an orthogonal-aminoacyl-tRNA synthetase that charges an orthogonal-tRNA with a noncanonical amino acid are also provided. For example, methods include subjecting a population of cells of a first species to a selection, where the cells each comprise: 1) a member of a plurality of aminoacyl-tRNA synthetases (aaRSs or RSs), (e.g., the plurality of RSs can include mutant RSs, RSs derived from a species other than a first species or both mutant RSs and RSs derived from a species other than a first species); 2) the orthogonal-tRNA (orthogonal-tRNA) (e.g., from one or more species); and 3) a polynucleotide that encodes a positive selection marker and comprises at least one selector codon. Cells (e.g., a host cell) are selected or screened for those that show an enhancement in suppression efficiency compared to cells lacking or having a reduced amount of the member of the plurality of RSs. These selected/screened cells comprise an active RS that aminoacylates the orthogonal-tRNA. An orthogonal aminoacyl-tRNA synthetase identified by the method is also a feature of the invention.

Methods producing tRNA-RS pairs that are orthogonal to endogenous tRNA-RS pairs as well as to mutant tRNA-RS pairs are also contemplated, and at least one method of determining such “dual” or “mutual” orthogonality is presented in this disclosure.

The invention also provides compositions that include proteins, where the proteins comprise a noncanonical amino acid. In some embodiments, the protein comprises an amino acid sequence that is at least 75% identical to that of a known protein, e.g., a therapeutic protein, a diagnostic protein, an industrial enzyme, or portions thereof. Optionally, the composition comprises a pharmaceutically acceptable carrier.

As described herein, the invention provides for polynucleotide sequences encoding, e.g., orthogonal-tRNAs and orthogonal-RSs, and polypeptide amino acid sequences, e.g., orthogonal-RSs, and, e.g., compositions, systems and methods comprising said polynucleotide or polypeptide sequences. One of skill in the art will appreciate that the invention is not limited to those sequences disclosed herein. One of skill will appreciate that the invention also provides many related sequences with the functions described herein, e.g., polynucleotides and polypeptides encoding conservative variants of an orthogonal-RS disclosed herein. General methodology for the construction and analysis of orthogonal synthetase species (orthogonal-RS) that are able to aminoacylate an orthogonal-tRNA with a noncanonical amino acid are known in the art.

Polynucleotides of the invention include those that encode proteins or polypeptides of interest of the invention with one or more selector codons (e.g., one having an ochre, opal, or four-base codon and one having an amber codon). Similarly, an artificial nucleic acid that hybridizes to a polynucleotide under highly stringent conditions over substantially the entire length of the nucleic acid (and is other than a naturally occurring polynucleotide) is a polynucleotide of the invention. In one embodiment, a composition includes a polypeptide of the invention and an excipient (e.g., buffer, water, pharmaceutically acceptable excipient, etc.). The invention also provides an antibody or antisera specifically immunoreactive with a polypeptide of the invention. As discussed herein, an artificial polynucleotide is a polynucleotide that is man made and is not naturally occurring.

A polynucleotide of the invention also includes an artificial polynucleotide that is, e.g., at least 75%, at least 80%, at least 90%, at least 95%, at least 98% or more identical to that of a naturally occurring tRNA, (but is other than a naturally occurring tRNA). A polynucleotide also includes an artificial polynucleotide that is, e.g., at least 75%, at least 80%, at least 90%, at least 95%, at least 98% or more identical (but not 100% identical) to that of a naturally occurring tRNA.

In some embodiments, a vector (e.g., a plasmid, a cosmid, a phage, a virus, etc.) comprises a polynucleotide of the invention. In one embodiment, the vector is an expression vector. In another embodiment, the expression vector includes a promoter operably linked to one or more of the polynucleotides of the invention. In another embodiment, a cell comprises a vector that includes a polynucleotide of the invention.

One of skill will also appreciate that many variants of the disclosed sequences are included in the invention. For example, conservative variations of the disclosed sequences that yield a functionally identical sequence are included in the invention. Variants of the nucleic acid polynucleotide sequences, wherein the variants hybridize to at least one disclosed sequence, are considered to be included in the invention. Unique subsequences of the sequences disclosed herein, as determined by, e.g., standard sequence comparison techniques, are also included in the invention.

Owing to the degeneracy of the genetic code, “silent substitutions” (i.e., substitutions in a nucleic acid sequence which do not result in an alteration in an encoded polypeptide) are an implied feature of every nucleic acid sequence that encodes an amino acid sequence. Similarly, “conservative amino acid substitutions,” where one or a limited number of amino acids in an amino acid sequence are substituted with different amino acids with highly similar properties, are also readily identified as being highly similar to a disclosed construct. Such conservative variations of each disclosed sequence are a feature of the present invention.

“Conservative variations” of a particular nucleic acid sequence refers to those nucleic acids which encode identical or essentially identical amino acid sequences, or, where the nucleic acid does not encode an amino acid sequence, to essentially identical sequences. One of skill will recognize that individual substitutions, deletions or additions which alter, add or delete a single amino acid or a small percentage of amino acids (typically less than 5%, more typically less than 4%, 2% or 1%) in an encoded sequence are “conservatively modified variations” where the alterations result in the deletion of an amino acid, addition of an amino acid, or substitution of an amino acid with a chemically similar amino acid. Thus, “conservative variations” of a listed polypeptide sequence of the present invention include substitutions of a small percentage, typically less than 5%, more typically less than 2% or 1%, of the amino acids of the polypeptide sequence, with an amino acid of the same conservative substitution group. Finally, the addition of sequences which do not alter the encoded activity of a nucleic acid molecule, such as the addition of a non-functional sequence, is a conservative variation of the basic nucleic acid.

Conservative substitution tables providing functionally similar amino acids are well known in the art, where one amino acid residue is substituted for another amino acid residue having similar chemical properties (e.g., aromatic side chains or positively charged side chains), and therefore does not substantially change the functional properties of the polypeptide molecule. The following table sets forth example groups that contain natural amino acids of like chemical properties, where substitutions within a group are “conservative substitutions.”

Conservative Amino Acid Substitutions

| Nonpolar | | | | |
|---|---|---|---|---|
| and/or | | | | |
| Aliphatic | Polar, | | Positively | Negatively |
| Side. | Unclaimed | Aromatic | Charged | Charged |
| Chains | Side Chains | Side Chains | Side Chains | Side Chains |
| Glycine | Serine | Phenylalanine | Lysine | Aspartate |
| Alanine | Threonine | Tyrosine | Arginine | Glutamate |
| Valine | Cysteine | Tryptophan | Histidine | |
| Leucine | Methionine | | | |
| Isoleucine | Asparagine | | | |
| Proline | Glutamine | | | |

Comparative hybridization can be used to identify nucleic acids of the invention, including conservative variations of nucleic acids of the invention, and this comparative hybridization method is a preferred method of distinguishing nucleic acids of the invention. In addition, target nucleic acids which hybridize to a nucleic acid under high, ultra-high and ultra-ultra high stringency conditions are a feature of the invention. Examples of such nucleic acids include those with one or a few silent or conservative nucleic acid substitutions as compared to a given nucleic acid sequence.

A test nucleic acid is said to specifically hybridize to a probe nucleic acid when it hybridizes at least 50% as well to the probe as to the perfectly matched complementary target, i.e., with a signal to noise ratio at least half as high as hybridization of the probe to the target under conditions in which the perfectly matched probe binds to the perfectly matched complementary target with a signal to noise ratio that is at least about 5-10 times as high as that observed for hybridization to any of the unmatched target nucleic acids.

Nucleic acids “hybridize” when they associate, typically in solution. Nucleic acids hybridize due to a variety of well characterized physico-chemical forces, such as hydrogen bonding, solvent exclusion, base stacking and the like. An extensive guide to the hybridization of nucleic acids is found in Tijssen (1993) Laboratory Techniques in Biochemistry and Molecular Biology—Hybridization with Nucleic Acid Probes, Part I, Chapter 2, “Overview of principles of hybridization and the strategy of nucleic acid probe assays,” (Elsevier, N.Y.), as well as in Current Protocols in Molecular Biology, Ausubel et al., eds., Current Protocols, a joint venture between Greene Publishing Associates, Inc. and John Wiley & Sons, Inc., (supplemented through 2004); Hames and Higgins (1995) Gene Probes 1, IRL Press at Oxford University Press, Oxford, England, and Hames and Higgins (1995) Gene Probes 2, IRL Press at Oxford University Press, Oxford, England, provide details on the synthesis, labeling, detection and quantification of DNA and RNA, including oligonucleotides.

An example of stringent hybridization conditions for hybridization of complementary nucleic acids which have more than 100 complementary residues on a filter in a Southern or northern blot is 50% formalin with 1 mg of heparin at 42° C., with the hybridization being carried out overnight. An example of stringent wash conditions is a 0.2×SSC wash at 65° C. for 15 minutes (see, Sambrook et al., Molecular Cloning—A Laboratory Manual (3rd Ed.), Vol. 1-3, Cold Spring Harbor Laboratory, Cold Spring Harbor, N.Y., 2001, for a description of SSC buffer). Often the high stringency wash is preceded by a low stringency wash to remove background probe signal. An example low stringency wash is 2×SSC at 40° C. for 15 minutes. In general, a signal to noise ratio of 5× (or higher) than that observed for an unrelated probe in the particular hybridization assay indicates detection of a specific hybridization.

“Stringent hybridization wash conditions” in the context of nucleic acid hybridization experiments such as Southern and northern hybridizations are sequence dependent, and are different under different environmental parameters. An extensive guide to the hybridization of nucleic acids is found in Tijssen (1993) Laboratory Techniques in Biochemistry and Molecular Biology—Hybridization with Nucleic Acid Probes, Part I, Chapter 2, “Overview of principles of hybridization and the strategy of nucleic acid probe assays,” (Elsevier, N.Y.), Hames and Higgins (1995) Gene Probes 1, IRL Press at Oxford University Press, Oxford, England, and Hames and Higgins (1995) Gene Probes 2, IRL Press at Oxford University Press, Oxford, England. Stringent hybridization and wash conditions can easily be determined empirically for any test nucleic acid. For example, in determining stringent hybridization and wash conditions, the hybridization and wash conditions are gradually increased (e.g., by increasing temperature, decreasing salt concentration, increasing detergent concentration or increasing the concentration of organic solvents such as formalin in the hybridization or wash), until a selected set of criteria are met. For example, in highly stringent hybridization and wash conditions, the hybridization and wash conditions are gradually increased until a probe binds to a perfectly matched complementary target with a signal to noise ratio that is at least 5× as high as that observed for hybridization of the probe to an unmatched target.

“Very stringent” conditions are selected to be equal to the thermal melting point (Tm) for a particular probe. The Tm is the temperature (under defined ionic strength and pH) at which 50% of the test sequence hybridizes to a perfectly matched probe. For the purposes of the present invention, generally, “highly stringent” hybridization and wash conditions are selected to be about 5° C. lower than the Tm for the specific sequence at a defined ionic strength and pH.

“Ultra high-stringency” hybridization and wash conditions are those in which the stringency of hybridization and wash conditions are increased until the signal to noise ratio for binding of the probe to the perfectly matched complementary target nucleic acid is at least 10× as high as that observed for hybridization to any of the unmatched target nucleic acids. A target nucleic acid which hybridizes to a probe under such conditions, with a signal to noise ratio of at least ½ that of the perfectly matched complementary target nucleic acid is said to bind to the probe under ultra-high stringency conditions.

Similarly, even higher levels of stringency can be determined by gradually increasing the hybridization or wash conditions of the relevant hybridization assay. For example, those in which the stringency of hybridization and wash conditions are increased until the signal to noise ratio for binding of the probe to the perfectly matched complementary target nucleic acid is at least 10.times., 20.times., 50.times., 100.times., or 500.times. or more as high as that observed for hybridization to any of the unmatched target nucleic acids. A target nucleic acid which hybridizes to a probe under such conditions, with a signal to noise ratio of at least ½ that of the perfectly matched complementary target nucleic acid is said to bind to the probe under ultra-ultra-high stringency conditions.

Nucleic acids which do not hybridize to each other under stringent conditions are still substantially identical if the polypeptides which they encode are substantially identical. This occurs, e.g., when a copy of a nucleic acid is created using the maximum codon degeneracy permitted by the genetic code.

The terms “identical” or “percent identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described below (or other algorithms available to persons of skill) or by visual inspection.

The phrase “substantially identical,” in the context of two nucleic acids or polypeptides refers to two or more sequences or subsequences that have at least about 60%, about 80%, about 90-95%, about 98%, about 99% or more nucleotide or amino acid residue identity, when compared and aligned for maximum correspondence, as measured using a sequence comparison algorithm or by visual inspection. Such “substantially identical” sequences are typically considered to be “homologous,” without reference to actual ancestry. Preferably, the “substantial identity” exists over a region of the sequences that is at least about 50 residues in length, more preferably over a region of at least about 100 residues, and most preferably, the sequences are substantially identical over at least about 150 residues, or over the full length of the two sequences to be compared.

Proteins or protein sequences are “homologous” when they are derived, naturally or artificially, from a common ancestral protein or protein sequence. Similarly, nucleic acids or nucleic acid sequences are homologous when they are derived, naturally or artificially, from a common ancestral nucleic acid or nucleic acid sequence. For example, any naturally occurring nucleic acid can be modified by any available mutagenesis method to include one or more selector codons. When expressed, this mutagenized nucleic acid encodes a polypeptide comprising one or more noncanonical amino acids. The mutation process can, of course, additionally alter one or more standard codon, thereby changing one or more standard amino acid in the resulting mutant protein as well. Homology is generally inferred from sequence similarity between two or more nucleic acids or proteins (or sequences thereof). The precise percentage of similarity between sequences that is useful in establishing homology varies with the nucleic acid and protein at issue, but as little as 25% sequence similarity is routinely used to establish homology. Higher levels of sequence similarity, e.g., 30%, 40%, 50%, 60%, 70%, 80%, 90%, 95%, or 99% or more, can also be used to establish homology. Methods for determining sequence similarity percentages (e.g., BLASTP and BLASTN using default parameters) are described herein and are generally available.

For sequence comparison and homology determination, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith and Waterman, Adv. Appl. Math. 2:482 (1981), by the homology alignment algorithm of Needleman and Wunsch, J. Mol. Biol. 48:443 (1970), by the search for similarity method of Pearson and Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Current Protocols in Molecular Biology, Ausubel et al., eds., Current Protocols, a joint venture between Greene Publishing Associates, Inc. and John Wiley & Sons, Inc., supplemented through 2006).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990), J. Mol. Biol. 215:403-410). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff and Henikoff (1989) Proc. Natl. Acad. Sci. USA 89:10915).

In addition to calculating percent sequence identity, the BLAST algorithm also performs a statistical analysis of the similarity between two sequences (see, e.g., Karlin and Altschul (1993), Proc. Nat'l. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

Polynucleotide and polypeptides of the invention and used in the invention can be manipulated using molecular biological techniques. General texts which describe molecular biological techniques include Berger and Kimmel, Guide to Molecular Cloning Techniques, Methods in Enzymology volume 152, Academic Press, Inc., San Diego, Calif.; Sambrook et al., Molecular Cloning—A Laboratory Manual (3rd Ed.), Vol. 1-3, Cold Spring Harbor Laboratory, Cold Spring Harbor, N.Y., 2001, and Current Protocols in Molecular Biology, Ausubel et al., eds., Current Protocols, a joint venture between Greene Publishing Associates, Inc. and John Wiley & Sons, Inc., (supplemented through 2006). These texts describe mutagenesis, the use of vectors, promoters and many other relevant topics related to, e.g., the generation of genes that include selector codons for production of proteins that include noncanonical amino acids, orthogonal tRNAs, orthogonal synthetases, and pairs thereof.

Various types of mutagenesis are used in the invention, e.g., to mutate tRNA molecules, to produce libraries of tRNAs, to mutate synthetases, to produce libraries of synthetases, to insert selector codons that encode a noncanonical amino acids in a protein or polypeptide of interest. They include but are not limited to site-directed, random point mutagenesis, homologous recombination, DNA shuffling or other recursive mutagenesis methods, chimeric construction, mutagenesis using uracil containing templates, oligonucleotide-directed mutagenesis, phosphorothioate-modified DNA mutagenesis, mutagenesis using gapped duplex DNA or the like, or any combination thereof. Additional suitable methods include point mismatch repair, mutagenesis using repair-deficient host strains, restriction-selection and restriction-purification, deletion mutagenesis, mutagenesis by total gene synthesis, double-strand break repair, and the like. Mutagenesis, e.g., involving chimeric constructs, is also included in the present invention. In one embodiment, mutagenesis can be guided by known information of the naturally occurring molecule or altered or mutated naturally occurring molecule, e.g., sequence, sequence comparisons, physical properties, crystal structure or the like.

Host cells are genetically engineered (e.g., transformed, transduced or transfected) with the polynucleotides of the invention or constructs which include a polynucleotide of the invention, e.g., a vector of the invention, which can be, for example, a cloning vector or an expression vector. For example, the coding regions for the orthogonal tRNA, the orthogonal tRNA synthetase, and the protein to be derivatized are operably linked to gene expression control elements that are functional in the desired host cell. Typical vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both (e.g., shuttle vectors) and selection markers for both prokaryotic and eukaryotic systems. Vectors are suitable for replication or integration in prokaryotes, eukaryotes, or preferably both. See Giliman and Smith, Gene 8:81 (1979); Roberts, et al., Nature, 328:731 (1987); Schneider et al., Protein Expr. Purif. 6435:10 (1995); Berger and Kimmel, Guide to Molecular Cloning Techniques, Methods in Enzymology volume 152, Academic Press, Inc., San Diego, Calif.; Sambrook et al., Molecular Cloning—A Laboratory Manual (3rd Ed.), Vol. 1-3, Cold Spring Harbor Laboratory, Cold Spring Harbor, N.Y., 2001, and Current Protocols in Molecular Biology, Ausubel et al., eds., Current Protocols, a joint venture between Greene Publishing Associates, Inc. and John Wiley & Sons, Inc., (supplemented through 2006). The vector can be, for example, in the form of a plasmid, a bacterium, a virus, a naked polynucleotide, or a conjugated polynucleotide. The vectors are introduced into cells or microorganisms by standard methods including electroporation (From et al. (1985), Proc. Natl. Acad. Sci. USA 82, 5824), infection by viral vectors, high velocity ballistic penetration by small particles with the nucleic acid either within the matrix of small beads or particles, or on the surface (Klein et al. (1987), Nature 327:70-73), or the like.

Bacteria and bacteriophage useful for cloning are widely known to one of skill in the art, and are available from a variety of sources. See, for example, the American Type Culture Collection (ATCC; Manassas, Va.) and The ATCC Catalogue of Bacteria and Bacteriophage (1996) Ghema et al. (eds) published by the ATCC. Additional basic procedures for sequencing, cloning and other aspects of molecular biology and underlying theoretical considerations are also found in Sambrook et al., Molecular Cloning—A Laboratory Manual (3rd Ed.), Vol. 1-3, Cold Spring Harbor Laboratory, Cold Spring Harbor, N.Y., 2001, and Current Protocols in Molecular Biology, Ausubel et al., eds., Current Protocols, a joint venture between Greene Publishing Associates, Inc. and John Wiley & Sons, Inc., (supplemented through 2006), and in Watson et al. (1992) Recombinant DNA Second Edition Scientific American Books, NY. In addition, essentially any nucleic acid (and virtually any labeled nucleic acid, whether standard or non-standard) can be custom or standard ordered from any of a variety of commercial sources, such as the Midland Certified Reagent Company (Midland, Tex.), The Great American Gene Company (Ramona, Calif.), ExpressGen Inc. (Chicago, Ill.), Operon Technologies Inc. (Alameda, Calif.) and many others.

The engineered host cells can be cultured in conventional nutrient media modified as appropriate for such activities as, for example, screening steps, activating promoters or selecting transformants. These cells can optionally be cultured into transgenic organisms. Other useful references, e.g., for cell isolation and culture (e.g., for subsequent nucleic acid isolation) include Freshney (1994) Culture of Animal Cells, a Manual of Basic Technique, Third Edition, Wiley-Liss, New York and the references cited therein; Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg N.Y.) and Atlas and Parks (eds) The Handbook of Microbiological Media (1993) CRC Press, Boca Raton, Fla.

In some embodiments, the orthogonal-RS comprises a bias for the aminoacylation of the cognate orthogonal-tRNA over any endogenous tRNA in an expression system. The relative ratio between orthogonal-tRNA and endogenous tRNA that is charged by the orthogonal-RS, when the orthogonal-tRNA and orthogonal-RS are present at equal molar concentrations, is greater than 1:1, preferably at least about 2:1, more preferably 5:1, still more preferably 10:1, yet more preferably 20:1, still more preferably 50:1, yet more preferably 75:1, still more preferably 95:1, 98:1, 99:1, 100:1, 500:1, 1,000:1, 5,000:1 or higher.

The invention also provides compositions that include proteins, where the proteins comprise a noncanonical amino acid. In some embodiments, the protein comprises an amino acid sequence that is at least 75% identical to that of a therapeutic protein, a diagnostic protein, an industrial enzyme, or portion thereof.

A cell of the invention provides the ability to synthesize proteins that comprise noncanonical amino acids in large useful quantities. In some aspects, the composition optionally includes, e.g., at least 10 micrograms, at least 50 micrograms, at least 75 micrograms, at least 100 micrograms, at least 200 micrograms, at least 250 micrograms, at least 500 micrograms, at least 1 milligram, at least 10 milligrams or more of the protein that comprises a noncanonical amino acid, or an amount that can be achieved with in vivo protein production methods (details on recombinant protein production and purification are provided herein). In another aspect, the protein is optionally present in the composition at a concentration of, e.g., at least 10 micrograms of protein per liter, at least 50 micrograms of protein per liter, at least 75 micrograms of protein per liter, at least 100 micrograms of protein per liter, at least 200 micrograms of protein per liter, at least 250 micrograms of protein per liter, at least 500 micrograms of protein per liter, at least 1 milligram of protein per liter, or at least 10 milligrams of protein per liter or more, in, e.g., a cell lysate, a buffer, a pharmaceutical buffer, or other liquid suspension (e.g., in a volume of, e.g., anywhere from about 1 mL to about 100 L). The production of large quantities (e.g., greater that that typically possible with other methods, e.g., in vitro translation) of a protein in a cell including at least two different noncanonical amino acids is a feature of the invention.

The incorporation of a noncanonical amino acid can be done to, e.g., tailor changes in protein structure or function, e.g., to change size, acidity, nucleophilicity, hydrogen bonding, hydrophobicity, accessibility of protease target sites, target to a moiety (e.g., for a protein array), incorporation of labels or reactive groups, etc. Proteins that include a noncanonical amino acid can have enhanced or even entirely new catalytic or physical properties. For example, the following properties are optionally modified by inclusion of a noncanonical amino acid into a protein: toxicity, biodistribution, structural properties, spectroscopic properties, chemical or photochemical properties, catalytic ability, half-life (e.g., serum half-life), ability to react with other molecules, e.g., covalently or noncovalently, and the like. The compositions including proteins that include at least two different noncanonical amino acids are useful for, e.g., novel therapeutics, diagnostics, catalytic enzymes, industrial enzymes, binding proteins (e.g., antibodies), and e.g., the study of protein structure and function. See, e.g., Dougherty, (2000) Noncanonical Amino Acids as Probes of Protein Structure and Function, Current Opinion in Chemical Biology, 4:645-652.

In some aspects of the invention, a composition includes at least one protein with at least at least two, e.g., at least three, at least four, at least five, at least six, at least seven, at least eight, at least nine, or at least ten or more noncanonical amino acids. The noncanonical amino acids can be the same or different, e.g., there can be 2, 3, 4, 5, 6, 7, 8, 9, or 10 or more different sites in the protein that comprise 2, 3, 4, 5, 6, 7, 8, 9, or 10 or more different noncanonical amino acids. In another aspect, a composition includes a protein with at least one, but fewer than all, of a particular amino acid present in the protein is a noncanonical amino acid. For a given protein with at least two different noncanonical amino acids, the noncanonical amino acids can be identical or different (e.g., the protein can include two or more different types of noncanonical amino acids, or can include two of the same noncanonical amino acid), but typically they are different. For a given protein with more than two noncanonical amino acids, the noncanonical amino acids can be the same, different or a combination of a multiple noncanonical amino acid of the same kind with at least one different noncanonical amino acid.

Essentially any protein (or portion thereof) that includes a noncanonical amino acid (and any corresponding coding nucleic acid, e.g., which includes one or more selector codons) can be produced using the compositions and methods herein. No attempt is made to identify the hundreds of thousands of known proteins, any of which can be modified to include one or more noncanonical amino acid, e.g., by tailoring any available mutation methods to include one or more appropriate selector codon in a relevant translation system. Common sequence repositories for known proteins include GenBank EMBL, DDBJ and the NCBI. Other repositories can easily be identified by searching the internet.

Typically, the proteins are, e.g., at least 60%, at least 70%, at least 75%, at least 80%, at least 90%, at least 95%, or at least 99% or more identical to any available protein (e.g., a therapeutic protein, a diagnostic protein, an industrial enzyme, or portion thereof, and the like), and they comprise one or more noncanonical amino acid. Examples of therapeutic, diagnostic, and other proteins that can be modified to comprise one or more noncanonical amino acid can be found, but not limited to, those in International Publications WO 2004/094593, filed Apr. 16, 2004, entitled “EXPANDING THE EUKARYOTIC GENETIC CODE;” and, WO 2002/085923, entitled “IN VIVO INCORPORATION OF NONCANONICAL AMINO ACIDS.” Examples of therapeutic, diagnostic, and other proteins that can be modified to comprise one or more noncanonical amino acids include, but are not limited to, e.g., hirudin, Alpha-1 antitrypsin, Angiostatin, Antihemolytic factor, antibodies (further details on antibodies are found below), Apolipoprotein, Apoprotein, Atrial natriuretic factor, Atrial natriuretic polypeptide, Atrial peptides, C-X-C chemokines (e.g., T39765, NAP-2, ENA-78, Gro-a, Gro-b, Gro-c, IP-10, GCP-2, NAP-4, SDF-1, PF4, MIG), Calcitonin, CC chemokines (e.g., Monocyte chemoattractant protein-1, Monocyte chemoattractant protein-2, Monocyte chemoattractant protein-3, Monocyte inflammatory protein-1 alpha, Monocyte inflammatory protein-1 beta, RANTES, 1309, R83915, R91733, HCC1, T58847, D31065, T64262), CD40 ligand, C-kit Ligand, Caspace, Collagen, Colony stimulating factor (CSF), Complement factor 5a, Complement inhibitor, Complement receptor 1, cytokines, (e.g., epithelial Neutrophil Activating Peptide-78, GRO-alpha/MGSA, GRO-beta, GRO-gamma, MIP-1-alpha, MIP-1-delta, MCP-1), Epidermal Growth Factor (EGF), Erythropoietin (“EPO”), Exfoliating toxins A and B, Factor IX, Factor VII, Factor VIII, Factor X, Fibroblast Growth Factor (FGF), Fibrinogen, Fibronectin, G-CSF, GM-CSF, Glucocerebrosidase, Gonadotropin, growth factors, Hedgehog proteins (e.g., Sonic, Indian, Desert), Hemoglobin, Hepatocyte Growth Factor (HGF), Hirudin, Human serum albumin, Insulin, Insulin-like Growth Factor (IGF), interferons (e.g., IFN-alpha, IFN-beta, IFN-gamma), interleukins (e.g., IL-1, IL-2, IL-3, IL-4, IL-5, IL-6, IL-7, IL-8, IL-9, IL-10, IL-11, IL-12, etc.), Keratinocyte Growth Factor (KGF), Lactoferrin, leukemia inhibitory factor, Luciferase, Neurturin, Neutrophil inhibitory factor (NIF), oncostatin M, Osteogenic protein, Parathyroid hormone, PD-ECSF, PDGF, peptide hormones (e.g., Human Growth Hormone), Pleiotropin, Procaspace-3, Procaspace-9, Protein A, Protein G, Pyrogenic exotoxins A, B, and C, Relaxin, Renin, SCF, Soluble complement receptor I, Soluble I-CAM 1, Soluble interleukin receptors (IL-1, 2, 3, 4, 5, 6, 7, 9, 10, 11, 12, 13, 14, 15), Soluble TNF receptor, Somatomedin, Somatostatin, Somatotropin, Streptokinase, Superantigens, i.e., Staphylococcal enterotoxins (SEA, SEB, SEC1, SEC2, SEC3, SED, SEE), Superoxide dismutase (SOD), Toxic shock syndrome toxin (TSST-1), Thymosin alpha 1, Tissue plasminogen activator, Tumor necrosis factor beta (TNF beta), Tumor necrosis factor receptor (TNFR), Tumor necrosis factor-alpha (TNF alpha), Vascular Endothelial Growth Factor (VEGEF), Urokinase and many others.

One class of proteins that can be made using the compositions and methods for in vivo incorporation of noncanonical amino acids described herein includes transcriptional modulators or a portion thereof. Example transcriptional modulators include genes and transcriptional modulator proteins that modulate cell growth, differentiation, regulation, or the like. Transcriptional modulators are found in prokaryotes, viruses, and eukaryotes, including fungi, plants, yeasts, insects, and animals, including mammals, providing a wide range of therapeutic targets. It will be appreciated that expression and transcriptional activators regulate transcription by many mechanisms, e.g., by binding to receptors, stimulating a signal transduction cascade, regulating expression of transcription factors, binding to promoters and enhancers, binding to proteins that bind to promoters and enhancers, unwinding DNA, splicing pre-mRNA, polyadenylating RNA, and degrading RNA.

One class of proteins of the invention (e.g., proteins with one or more noncanonical amino acids) include biologically active proteins such as hirudin, cytokines, inflammatory molecules, growth factors, their receptors, and oncogene products, e.g., interleukins (e.g., IL-1, IL-2, IL-8, etc.), interferons, FGF, IGF-I, IGF-II, FGF, PDGF, TNF, TGF-alpha, TGF-beta, EGF, KGF, SCF/c-Kit, CD40UCD40, VLA-4/VCAM-1, ICAM-1/LFA-1, and hyalurin/CD44; signal transduction molecules and corresponding oncogene products, e.g., Mos, Ras, Raf, and Met; and transcriptional activators and suppressors, e.g., p53, Tat, Fos, Myc, Jun, Myb, Rel, and steroid hormone receptors such as those for estrogen, progesterone, testosterone, aldosterone, the LDL receptor ligand and corticosterone.

Enzymes (e.g., industrial enzymes) or portions thereof with at least two different noncanonical amino acids are also provided by the invention. Examples of enzymes include, but are not limited to, e.g., amidases, amino acid racemases, acylases, dehalogenases, dioxygenases, diarylpropane peroxidases, epimerases, epoxide hydrolases, esterases, isomerases, kinases, glucose isomerases, glycosidases, glycosyl transferases, haloperoxidases, monooxygenases (e.g., p450s), lipases, lignin peroxidases, nitrile hydratases, nitrilases, proteases, phosphatases, subtilisins, transaminase, and nucleases.

Many of these proteins are commercially available (See, e.g., the Sigma BioSciences 2002 catalogue and price list), and the corresponding protein sequences and genes and, typically, many variants thereof, are well-known (see, e.g., Genbank). Any of them can be modified by the insertion of one or more noncanonical amino acid according to the invention, e.g., to alter the protein with respect to one or more therapeutic, diagnostic or enzymatic properties of interest. Examples of therapeutically relevant properties include serum half-life, shelf half-life, stability, immunogenicity, therapeutic activity, detectability (e.g., by the inclusion of reporter groups (e.g., labels or label binding sites) in the noncanonical amino acids), reduction of LD50 or other side effects, ability to enter the body through the gastric tract (e.g., oral availability), or the like. Examples of diagnostic properties include shelf half-life, stability, diagnostic activity, detectability, or the like. Examples of relevant enzymatic properties include shelf half-life, stability, enzymatic activity, production capability, or the like.

A variety of other proteins can also be modified to include one or more noncanonical amino acid using compositions and methods of the invention. For example, the invention can include substituting one or more natural amino acids in one or more vaccine proteins with a noncanonical amino acid, e.g., in proteins from infectious fungi, e.g., Aspergillus, Candida species; bacteria, particularly E. coli, which serves a model for pathogenic bacteria, as well as medically important bacteria such as Staphylococci (e.g., aureus), or Streptococci (e.g., pneumoniae); protozoa such as sporozoa (e.g., Plasmodia), rhizopods (e.g., Entamoeba) and flagellates (Trypanosoma, Leishmania, Trichomonas, Giardia, etc.); viruses such as (+) RNA viruses (examples include Poxviruses e.g., vaccinia; Picornaviruses, e.g., polio; Togaviruses, e.g., rubella; Flaviviruses, e.g., HCV; and Coronaviruses), (−) RNA viruses (e.g., Rhabdoviruses, e.g., VSV; Paramyxovimses, e.g., RSV; Orthomyxovimses, e.g., influenza; Bunyaviruses; and Arenaviruses), dsDNA viruses (Reoviruses, for example), RNA to DNA viruses, i.e., Retroviruses, e.g., HIV and HTLV, and certain DNA to RNA viruses such as Hepatitis B.

Agriculturally related proteins such as insect resistance proteins (e.g., the Cry proteins), starch and lipid production enzymes, plant and insect toxins, toxin-resistance proteins, Mycotoxin detoxification proteins, plant growth enzymes (e.g., Ribulose 1,5-Bisphosphate Carboxylase/Oxygenase, “RUBISCO”), lipoxygenase (LOX), and Phosphoenolpyruvate (PEP) carboxylase are also suitable targets for noncanonical amino acid modification.

In some embodiments, the protein or polypeptide of interest (or portion thereof) in the methods or compositions of the invention is encoded by a nucleic acid. Typically, the nucleic acid comprises at least one selector codon, at least two selector codons, at least three selector codons, at least four selector codons, at least five selector codons, at least six selector codons, at least seven selector codons, at least eight selector codons, at least nine selector codons, ten or more selector codons.

Genes coding for proteins or polypeptides of interest can be mutagenized using methods well-known to one of skill in the art and described herein under “Mutagenesis and Other Molecular Biology Techniques” to include, e.g., one or more selector codon for the incorporation of a noncanonical amino acid. For example, a nucleic acid for a protein of interest is mutagenized to include one or more selector codon, providing for the insertion of the one or more noncanonical amino acids. The invention includes any such variant, e.g., mutant, versions of any protein, e.g., including at least two different noncanonical amino acids.

To make a protein that includes at least two different noncanonical amino acids, one can use host cells and organisms that are adapted for the in vivo incorporation of the noncanonical amino acid via orthogonal tRNA/RS pairs. Host cells are genetically engineered (e.g., transformed, transduced or transfected) with one or more vectors that express the orthogonal tRNA-tRNA synthetase pair of interest, and a vector that encodes the protein to be derivatized. Each of these components can be on the same vector, or each can be on a separate vector, or two components can be on one vector and the third component on a secondvector. The vector can be, for example, in the form of a plasmid, a bacterium, a virus, a naked polynucleotide, or a conjugated polynucleotide.

Because the polypeptides of the invention provide a variety of new polypeptide sequences (e.g., polypeptides comprising noncanonical amino acids in the case of proteins synthesized in the translation systems herein, or, e.g., in the case of the novel synthetases, novel sequences of standard amino acids), the polypeptides also provide new structural features which can be recognized, e.g., in immunological assays. The generation of antisera, which specifically bind the polypeptides of the invention, as well as the polypeptides which are bound by such antisera, are a feature of the invention. The term “antibody,” as used herein, includes, but is not limited to a polypeptide substantially encoded by an immunoglobulin gene or immunoglobulin genes, or fragments thereof which specifically bind and recognize an analyte (antigen). Examples include polyclonal, monoclonal, chimeric, and single chain antibodies, and the like. Fragments of immunoglobulins, including Fab fragments and fragments produced by an expression library, including phage display, are also included in the term “antibody” as used herein. See, e.g., Paul, Fundamental Immunology, 4th Ed., 1999, Raven Press, New York, for antibody structure and terminology.

In order to produce antisera for use in an immunoassay, one or more of the immunogenic polypeptides is produced and purified as described herein. For example, recombinant protein can be produced in a recombinant cell. An inbred strain of mice (used in this assay because results are more reproducible due to the virtual genetic identity of the mice) is immunized with the immunogenic protein(s) in combination with a standard adjuvant, such as Freund's adjuvant, and a standard mouse immunization protocol (see, e.g., Harlow and Lane (1988) Antibodies, A Laboratory Manual, Cold

Spring Harbor Publications, New York, for a standard description of antibody generation, immunoassay formats and conditions that can be used to determine specific immunoreactivity. Additional details on proteins, antibodies, antisera, etc. can be found in International Publication Numbers WO 2004/094593, entitled “EXPANDING THE EUKARYOTIC GENETIC CODE;” WO 2002/085923, entitled “IN VIVO INCORPORATION OF NONCANONICAL AMINO ACIDS;” WO 2004/035605, entitled “GLYCOPROTEIN SYNTHESIS;” and WO 2004/058946, entitled “PROTEIN ARRAYS.”

In some embodiments, a step, reagent, reactive solid, base material, supplementary material, secondary reagent, contaminant, reactor, component, etc., can optionally be excluded. In some embodiments, for example, nitrate removal is excluded. In some embodiments, selenocyanate removal is excluded. Further, any embodiment herein reciting “comprising” can optionally recite instead “consist of” or “consist essentially of.”

It will be understood that aspects of embodiments provided in this disclosure can be used singly or in combination. Disclosed are materials, compositions, systems, and components that can be used for, can be used in conjunction with, can be used in preparation for, or are products of the disclosed methods and compositions. These and other materials are disclosed herein and it is understood that when combinations, subsets, interactions, groups, etc., of these materials are disclosed that while specific reference of each various individual and collective combinations and permutations of these compounds can not be explicitly disclosed, each is specifically contemplated and described herein. For example, if a method is disclosed and discussed and a number of modifications that can be made are discussed, each and every combination and permutation of the method, and the modifications that are possible, are specifically contemplated unless specifically indicated to the contrary. Likewise, any subset or combination of these is also specifically contemplated and disclosed. This concept applies to all aspects of this disclosure including, but not limited to, steps in methods using the disclosed compositions and components of systems. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed. It is therefore contemplated that any embodiment discussed in this specification can be implemented with respect to any method, system, host cell, polynucleotide, protein, cell, vector, etc., described herein, and vice versa.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value. In any embodiment discussed in the context of a numerical value used in conjunction with the term “about,” it is specifically contemplated that the term about can be omitted.

Following long-standing patent law, the words “a” and “an,” when used in conjunction with the word “comprising” in the claims or specification, denotes one or more, unless specifically noted.

The following examples are provided for the purpose of illustrating, not limiting, the invention.

### EXAMPLES

### Example 1

This Example describes the development of a facile system for genetic incorporation of two different noncanonical amino acids into one protein in Escherichia coli.

Introduction

Since the first report in 1998, genetic incorporation of noncanonical amino acids (NAAs) into proteins at amber UAG codons in living cells using bio-orthogonal aminoacyl-tRNA synthetase (aaRS)-amber suppressor tRNA (tRNA_{CUA}) pairs has flourished. Evolved Methanococcus jannaschii tyrosyl-tRNA synthetase (MjTyrRS)-MjtRNA(Tyr/CUA) pairs together with the naturally occurring wild-type or evolved pyrrolysyl-tRNA synthetase (PylRS)-tRNA^{Pyl} (pylT) pairs have enabled the incorporation of more than 30 NAAs into proteins in E. coli. Although the incorporation of these NAAs, which have unique chemical properties and reactivities, into proteins has dramatically increased our ability to manipulate protein structure and function, major limitations still exist for the technique. Namely, the technique in general only allows the incorporation of a single NAA into a single protein because the amber codon is the only one available for the incorporation of NAAs and the nonsense suppression rate in living cells is low. The incorporation of up to three N^{ε}-acetyl-L-lysines (AcKs) at different amber mutation sites of GFP_{UV} in E. coli has been previously demonstrated using an enhanced amber suppression condition. However, in order to incorporate two or more different NAAs into a single protein, the use of at least one additional blank codon is required. The aaRS-tRNA pairs that correspond to the additional blank codons must be orthogonal with respect to the amber suppressor aaRS-tRNA pairs and the endogenous aaRS-tRNA pairs to predictably and routinely incorporate the two or more NAAs into the single protein. Furthermore, suppression of each stop/blank codon must be sufficiently efficient to avoid the termination of translation and, thus avoid problems with low yield.

It is demonstrated herein that the PylRS-pylT pair can be mutated to suppress the ochre UAA codon and the mutated pair can be coupled with an evolved MjTyrRS-MjtRNA (Tyr/CUA) pair to efficiently incorporate two different NAAs at two defined sites of a single protein in E. coli by both amber and ochre suppressions. This development greatly expands the diversity of modifications that can be introduced into proteins.

Results/Discussion

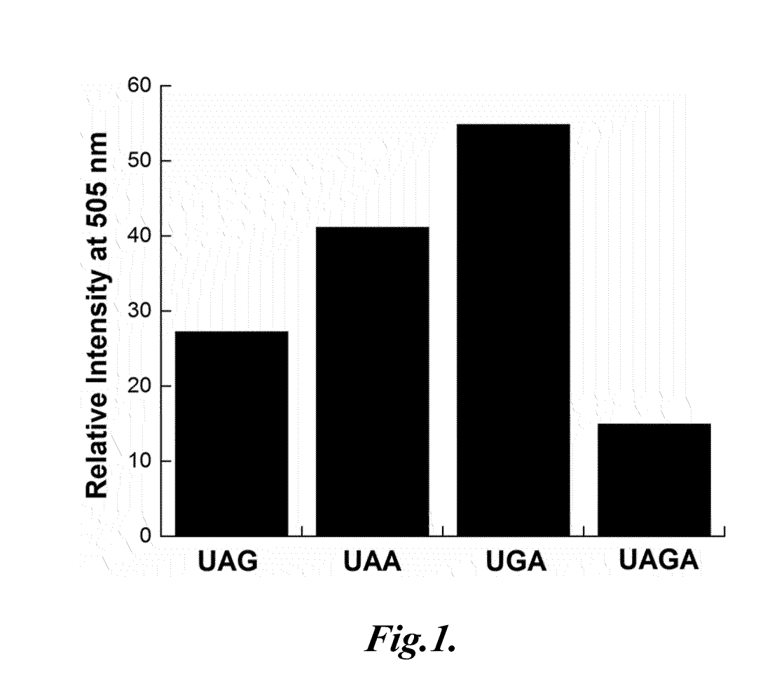

The PylRS-pylT pair and the MjTyrRS-MjtRNA (Tyr/CUA) pair were used together to incorporate two different NAAs into a single protein because of their proven efficiency in NAA incorporation. To use both pairs in a single E. coli cell, it was first demonstrated that they are orthogonal to each other. When co-expressed in E. coli, either one of the aaRSs could not efficiently charge tRNA_{CUA} from the other pair with its cognate amino acid (N^{ε}-Boc-L-lysine for PylRS for PylRS and L-tyrosine for MjTyrRS) to suppress an amber mutation at position 112 of a chloramphenicol acetyltransferase gene to give detectable chloramphenicol resistance. It was then tested whether mutated pylT can be used to suppress other blank codons. Although naturally encoded by the UAG codon, it has been reported that pyrrolysine is not hardwired for cotranslational insertion at UAG codon positions. The crystal structure of the PylRS-pylT complex also revealed no direct interaction between PylRS and the anticodon region of pylT. Therefore, the potential influence of a mutation of the anticodon of pylT on its interaction with PylRS was addressed. Mutated PylRS-pylT pairs were generated to suppress a blank codon such as opal UGA codon, ochre UAA codon, or even a four-base codon UAGA. A pAcKRS-pylT-GFP1Amber plasmid from previous work was employed for this purpose. This plasmid contains genes coding a mutant PylRS (AcKRS) specific for AcK, pylT, and GFP_{UV}. The GFP_{UV} gene has one amber mutation corresponding to position 149 of the protein. Growing cells transformed with this plasmid in lysogeny broth (LB) medium supplemented with 5 mm AcK led to full-length GFP_{UV} expression that was easily detected by fluorescent emission of GFP_{UV} when excited (FIG. 1). When the anticodon of pylT was mutated to the complimentary one for opal codon (UGA), ochre codon (UAA), or a four-base UAGA codon, and the corresponding codons were introduced to the nucleic acid encoding position 149 of GFP_{UV} in the pAcKRS-pylT-GFP1Amber plasmid, cells transformed with the plasmid and grown in LB medium supplemented with 5 mm AcK also exhibited detectable GFP_{UV} expression for all three mutated pylTs (FIG. 1). The nucleic acid sequences for the pylT, and pylT with complementary anticodons for opal, ochre, and the four-base codons, are set forth herein as SEQ ID NOS:1-4, respectively. In comparison to wild-type pylT, both pylT that suppresses opal codon and pylT that suppresses ochre codon gave significantly higher suppression levels. This result indicates that both the PylRS-pylT_{UCA} pair and the PylRS-pylT_{UUA} pair can be used to efficiently incorporate NAAs into proteins at their corresponding suppressed codons. Furthermore, it demonstrates that it can be feasible to couple the PylRS-pylT_{UCA} (or pylT_{UUA}) pair together with an evolved MjTyrRS-MjtRNA(Tyr/CUA) pair to incorporate two different NAAs into a single protein in E. coli by both amber and opal (or ochre) suppressions. Although the suppression of opal mutation in GFP_{UV} by pylT_{UCA} led to a higher full-length GFP_{UV} expression level than in the case with the suppression of ochre mutation in GFP_{UV} by pylT_{UUA}, the ochre suppressor was utilized for further NAA incorporation because the anticodon of an opal suppressor can form a wobble pair with the UGG codon in mRNA and decrease the fidelity of tryptophan incorporation.

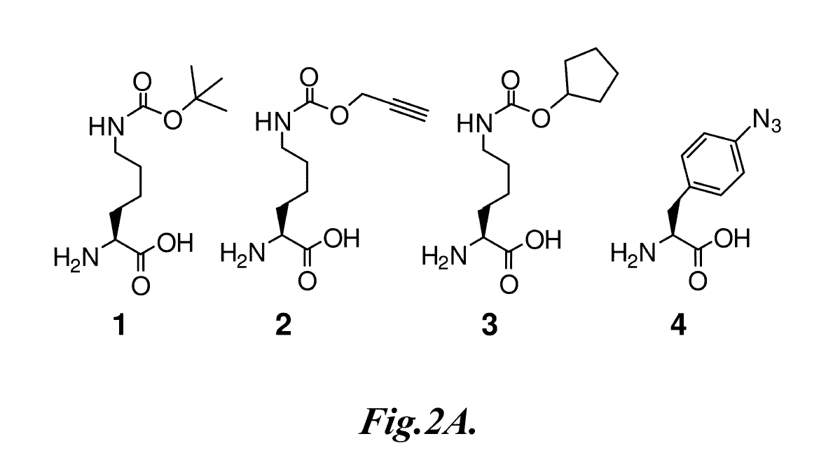

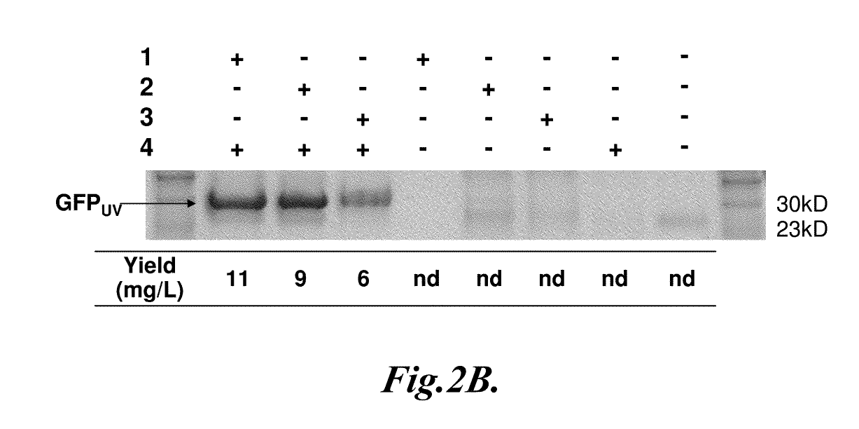

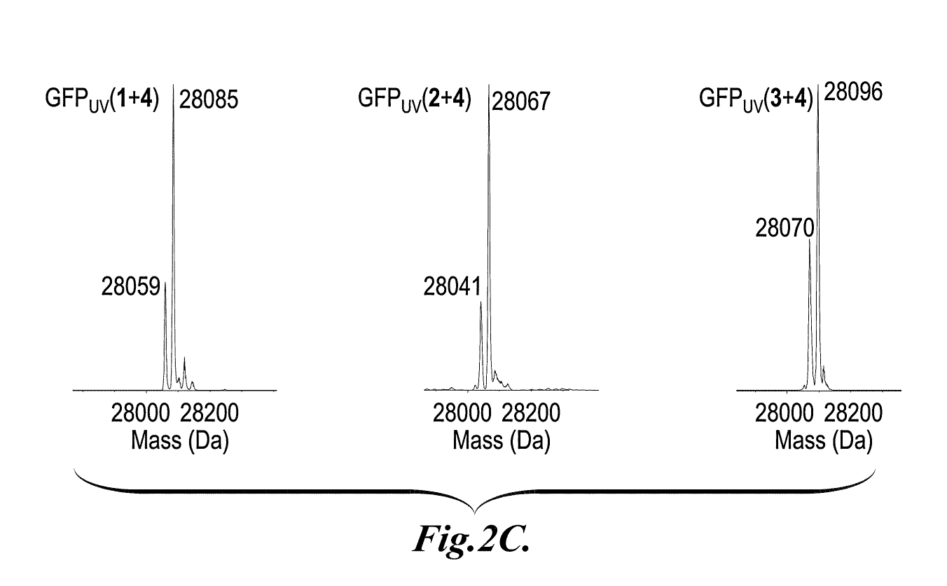

To demonstrate the utility of the PylRS-pylT_{UUA} pair together with an evolved MjTyrRS-MjtRNA(Tyr/CUA) pair to incorporate two different NAAs into a single protein in E. coli by both amber and ochre suppressions, two plasmids, pEVOL-AzFRS and pPylRS-pylT-GFP1TAG149TAA (see methods and material section below), were used to transform E. coli BL21 cells. The pEVOL-AzFRS plasmid contains genes encoding an optimized MjtRNA (Tyr/CUA) and two copies of an evolved MjTyrRS (AzFRS) specific for p-azido-L-phenylalanine (NAA #4, also referred to in this Example as 4; see FIG. 2A). This plasmid provides an enhanced amber suppression in E. coli. The pPylRS-pylT-GFP1TAG149TAA plasmid contains genes encoding wildtype M. mazei PylRS, pylT_{UUA}, and GFP_{UV}. The GFP_{UV} gene has an amber mutation at position 1, an ochre mutation at 149, an N-terminal Met-Ala leader dipeptide in front of the amber mutation, and an opal stop codon at the C-terminal end. The nucleic acid sequence for the GFP_{UV} “1TAG, 149TAA” gene is set forth as SEQ ID NO:5. The corresponding amino acid sequence is set forth as SEQ ID. NO:6, with “X” indicating noncanonical amino acids. It is noted that while the Met-Ala leader dipeptide listed in SEQ NO:6 shifts the place of the noncanonical amino acids to the 3^{rd} and 151^{st} residues, these positions are referred to herein as positions 1 and 149 of the GFP_{UV} polypeptide. Growing the transformed cells in 2YT medium supplemented with 1 mM N^{ε}-Boc-L-lysine (NAA #1, also referred to in this Example as 1; see FIG. 2A) and 1 mM NAA #4 afforded full-length GFP_{UV} with a yield of 11 mgL⁻¹ (FIG. 2B, lane 1). No cellular toxicity owing to strong amber and ochre suppressions was observed. Exclusion of either NAA from the medium led to no detectable full-length GFP_{UV} expression (FIG. 2A, lanes 4 and 7). The results indicate that the suppressions of amber and opal mutations are dependent on the presence of their corresponding NAAs. The ESI-MS of the purified full-length GFP_{UV} incorporated with NAA #4 at position 1 and NAA #1 at position 149 (GFP_{UV}(1+4)) confirmed the expected incorporations (FIG. 2C). The detected mass (28,085 Da) agrees within 70 parts per million with the calculated mass (28,083 Da) of full-length GFP_{UV}(1+4) without N-terminal methionine. The cleavage of N-terminal methionine from expressed GFP_{UV} in E. coli has been observed in related studies. A mass peak (28,059 Da) that is 26 Da smaller than the major peak is probably due to the decomposition of the azide group in NAA #4 to form the corresponding amine during ESI-MS analysis, which has been observed previously. As wild-type PylRS also charges pylT with N^{ε}-propargyloxycarbonyl-l-lysine (NAA #2, also referred to in this Example as 2; see FIG. 2A) and N^{ε}-cyclopentyloxycarbonyl-l-lysine (NAA #3, also referred to in this Example as 3; see FIG. 2A), the incorporation of either of these two NAAs together with NAA #4 into GFP_{UV} was also tested in cells transformed with pEVOL-AzFRS and pPylRS-pylT-GFP1TAG149TAA. Growing cells in 2YT medium supplemented with 1 mM NAA #2 (or NAA #3) and 1 mm NAA #4 afforded full-length GFP_{UV} in good yields (see FIG. 2B). No full-length GFP_{UV} expression was detected when only one NAA was present in the medium. ESI-MS analysis of the purified proteins confirmed the expected incorporations (GFP_{UV} incorporated with NAA #4 and NAA #2 (GFP_{UV}(2+4)): 28,065 Da (calculated), 28,067 Da (detected); GFP_{UV} incorporated with NAA #4 and NAA #3 (GFP_{UV}(3+4)): 28,095 Da (calculated), 28,096 Da (detected)). Similar decomposition of the azide group during MS analysis was observed in both cases (FIG. 2C).

As GFP_{UV}(2+4) contains both an alkyne group and an azide group, the feasibility of separately labeling this protein with different fluorescent dyes by performing click reactions on these two functional groups was tested. The reaction of GFP_{UV}(2+4) with 3-azido-7-hydroxycoumarin in the presence of a Cu^{I} catalyzed to a labeled GFP_{UV} that emitted strong blue fluorescence under long wavelength UV light (365 nm) after the protein was denatured and analyzed in a SDS-PAGE gel (not shown). The same labeling reaction between wild-type GFP_{UV} (wtGFP_{UV}) did not give any detectable blue fluorescence when excited. As both proteins were denatured prior to SDS-PAGE analysis, the endogenous fluorophore of GFP_{UV} was quenched and did not interfere with the analysis. The presence of protein in the gel was confirmed with Coomassie blue staining in the same SDS-PAGE gel. The similar click chemistry reaction was also carried out between GFP_{UV}(2+4) and a propargyl-conjugated fluorescein (also referred to in the Methods and Materials section as compound 6). The labeled protein emitted strong green fluorescence when excited at 365 nm (not shown). The control reaction on wtGFP_{UV} again yielded no detectable fluorescently labeled protein. These results demonstrate that both side-chain functional groups of NAA #2 and NAA #4 are active after their incorporation into proteins and can be used separately to effect site-specific protein modifications. As a large excess of 3-azido-7-hydroxycoumarin or propargyl-conjugated fluorescein relative to the protein were used during labeling experiments, the self-coupling reaction between the azide and alkyne groups from two GFP_{UV}(2+4) molecules was prevented. No GFP_{UV}(2+4) dimer was observed after the reactions.

To generalize the method, the feasibility of using evolved PylRS-pylT_{UUA} and MjTyrRS-MjtRNA(Tyr/CUA) pairs to genetically incorporate their cognate NAAs into one protein in E. coli was tested. The PylRS gene in the pPylRS-pylT-GFP1TAG149TAA plasmid was replaced with the AcKRS gene and then transformed E. coli BL21 cells with the modified plasmid together with pEVOL-AzFRS. Growing the transformed cells in 2YT medium supplemented with 1 mM NAA #4 and 5 mM AcK, also referred to in the Methods and Materials section as compound 12, led to full-length GFP_{UV} expression with a yield of 6.4 mgL⁻¹ (see Methods and Materials, section 7.2). Similarly, AzFRS in the pEVOL-AzFRS was replaced with an evolved MjTyrRS (sTyrRS) that is specific for O-sulfo-L-tyrosine (sTyr, also referred to in the Methods and Materials as compound 13) and used the resulting plasmid and pPylRS-pylT-GFP1TAG149TAA to transform E. coli BL21 cells. Growing cells in 2YT medium supplemented with 1 mM sTyr and 1 mm NAA #2 led to full-length GFP_{UV} expression with a yield of 0.4 mgL⁻¹ (see Methods and Materials, section 7.2). The low GFP_{UV} expression yield in this case is most likely due to the low efficiency of the evolved sTyrRS. In both cases, no full-length GFP_{UV} was expressed when only one NAA was provided in the media. These results indicate that the method described herein can be generally applied to combine any two evolved PylRS-pylT_{UUA} and MjTyrRS-MjtRNA(Tyr/CUA) pairs to genetically incorporate their cognate NAAs into a single protein in E. coli.

In summary, as described herein, a facile system for genetic incorporation of two different NAAs at two defined sites of a single protein in E. coli with surprisingly high protein production yields. This technique greatly expands the scope of potential applications that use the genetic incorporation NAA into proteins to expand the genetic code. This technique is useful numerous applications, such as to install a FRET pair to a protein for conformation and dynamics studies, to synthesize proteins with two different post-translational modifications for functional analysis, and to generate phage-displayed peptide libraries with the expanded diversity of the displayed peptides.

Methods and Materials

The construction of pPylRS-pylT-GFP1TAG149TAA and other plasmids all followed standard cloning and QuikChange site-directed mutagenesis procedures using Platinum Pfx (Invitrogen) and Pfu-Turbo (Stratagene) DNA polymerases. Sequences of all the constructed plasmids were verified by DNA sequencing. Specific details regarding plasmid construction, synthesis and characterization of NAAs and fluorescent dyes, GFP_{UV} expression, ESI-MS analysis and fluorescent labeling of expressed proteins, are provided below.

### 1. General Experimental Description

All reactions involving moisture sensitive reagents were conducted in oven-dried glassware under an argon atmosphere. Anhydrous solvents were obtained through standard laboratory protocols. Analytical thin-layer chromatography (TLC) was performed on Whatman SiO₂ 60 F-254 plates. Visualization was accomplished by UV irradiation at 254 nm or by staining with ninhydrin (0.3% w/v in glacial acetic acid/n-butyl alcohol 3:97). Flash column chromatography was performed with flash silica gel (particle size 32-63 μm) from Dynamic Adsorbents Inc. (Atlanta, Ga.).

Specific rotations of chiral compounds were obtained at the designated concentration and temperature on a Rudolph Research Analytical Autopol H polarimeter using a 0.5 dm cell. Proton and carbon NMR spectra were obtained on Varian 300 and 500 MHz NMR spectrometers. Chemical shifts are reported as δ values in parts per million (ppm) as referenced to the residual solvents: chloroform (7.27 ppm for and 77.23 ppm for ¹³C), methanol (3.31 ppm for ¹H and 49.15 ppm for ¹³C), or water (4.80 ppm for ¹H) (spectra not shown). A minimal amount of 1,4-dioxane was added as the reference standard (67.19 ppm for ¹³C) for carbon NMR spectra in deuterium oxide, and a minimal amount of sodium hydroxide pellet was added to the NMR sample to aid in the solvation of amino acids which have low solubility in deuterium oxide under neutral or acidic conditions. ¹H NMR spectra were tabulated as follows: chemical shift, multiplicity (s=singlet, bs=broad singlet, d=doublet, t=triplet, q=quartet, m=multiplet), number of protons, and coupling constant(s). Mass spectra were obtained at the Laboratory for Biological Mass Spectrometry at the Department of Chemistry, Texas A&M University (not shown).

3-Azido-7-hydroxycoumarin was synthesized as previously reported in K. Silvakumar, et al., Org. Lett. 6:4603 (2004). N^{ε}-Boc-L-lysine (NAA #1) and 4-azido-L-phenylalanine (NAA #4) were obtained from Chem-Impex International, Inc. (Wood Dale, Ill.). Cyclopentyl chloroformate was obtained from Amfinecom Inc. (Petersburg, Va.). Sulfonated bathophenanthroline sodium salt was obtained from GFS Chemicals (Powell, Ohio). All other reagents were obtained from commercial suppliers and used as received.

### 2. Synthesis of NAA #2, NAA #3, and Propargyl-Conjugated Fluorescein

### 2.1—Synthetic Schemes

NAA compounds #2 and #3 were synthesized in a divergent route (Scheme 1). Propargyl-conjugated fluorescein was synthesized in one step following the literature protocol for similar compounds (Scheme 2).

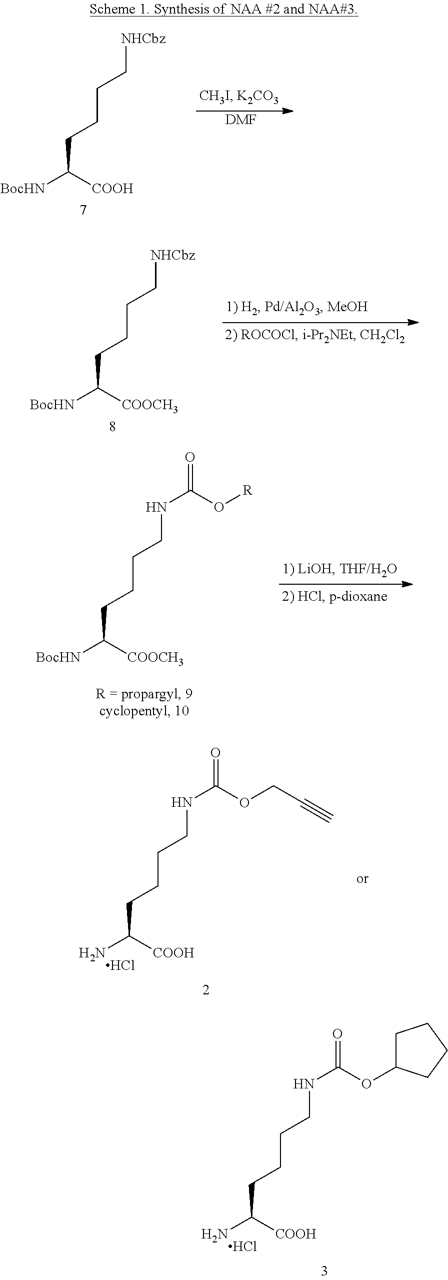

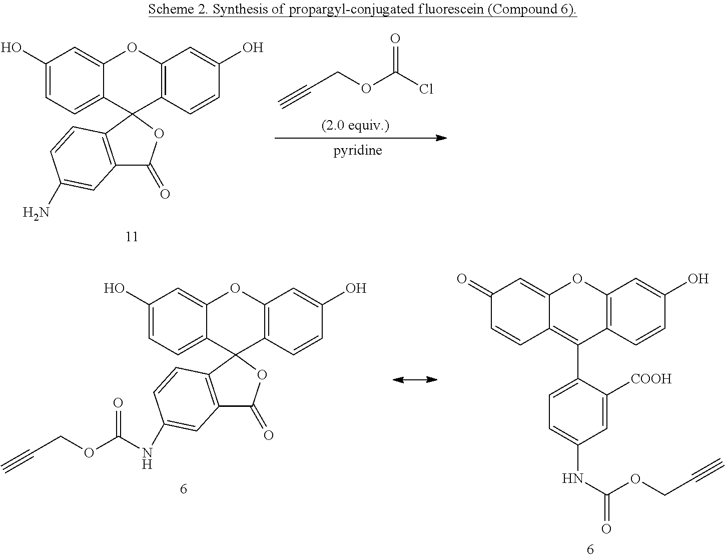

### 2.2—Boc-Lys(Z)-OMe (Compound 8)

To a suspension of Boc-Lys(Z)-OH (referred to in this Example as compound 7, or 7, 40.3 g, 0.106 mol) and potassium carbonate (27.6 g, 0.200 mol) in DMF (200 mL) was added iodomethane (9.90 mL, 0.159 mol), and the mixture was stirred at room temperature for 30 h. The mixture was filtered, and the filter cake was washed with ethyl acetate (50 mL), dissolved in water (100 mL), and extracted with ethyl acetate (100 mL×2). All the ethyl acetate solutions were combined with the filtrate, and the solution was evaporated under vacuum until most of the DMF has been removed. The residue was redissolved in ether (250 mL), washed with water (100 mL) and brine (50 mL), dried (Na₂SO₄), and evaporated to a compound referred to in this Example as compound 8 (41.8 g, quant.) as a yellow oil. The material was pure enough for the next-step reaction without further treatment.

A fraction of pure compound 8 was obtained by flash chromatography (EtOAc/hexanes, 1:5) for characterization: R_{f}=0.35 (EtOAc/hexanes, 1:2); [α]_{D}²² +6.1 (c 2.90, CHCl₃) (lit. [α]_{D}²⁰ +7.8 (c 3.93, CHCl₃)); ¹H NMR (CDCl₃, 500 MHz) δ 7.36-7.35 (m, 4H), 7.33-7.30 (m, 1H), 5.12 (d, 1H, J=8.5 Hz), 5.09 (s, 2H), 4.89 (bs, 1H), 4.30-4.26 (m, 1H), 3.73 (s, 3H), 3.19 (dt, 2H, J=6.7, 6.7 Hz), 1.80-1.77 (m, 1H), 1.68-1.60 (m, 1H), 1.55-1.49 (m, 2H), 1.43 (s, 9H), 1.39-1.33 (m, 2H); ¹³C NMR (CDCl₃, 125 MHz) δ 173.5, 156.6, 155.6, 136.7, 128.7, 128.32, 128.27, 80.1, 66.8, 53.3, 52.5, 40.8, 32.5, 29.5, 28.5, 22.5.

In order to check the optical purity of compound 8, a small amount of racemic 8 was obtained in the same way starting from racemic 7. The racemic sample was dissolved in isopropanol (10 mg/mL), filtered over 0.2 μm PTFE membrane filter (VWR), and injected (4 uL) onto the Shimadzu LC system equipped with a Chiralpak IB column. The sample was isocratically eluted with 10% isopropanol in hexanes and the peaks were monitored at 210 nm. The (S)-enantiomer was eluted at 12.72 min and the (R)-enantiomer was eluted at 11.59 min. A sample of 8 (10 mg/mL, 2 pt) was subsequently analyzed under the same conditions, and its e.e. was determined to be 100%.

### 2.3—N-α-Boc-N-β-propargyloxycarbonyl-L-lysine methyl ester (Compound 9)

A solution of compound 8 (3.61 g, 9.15 mmol) in methanol (100 mL) was hydrogenated under a H₂ balloon in the presence of palladium on alumina (10 wt. % Pd, 0.61 g, 0.57 mmol) at room temperature for 4 h, and TLC analysis showed a complete conversion. The mixture was then filtered over a pad of Celite and evaporated to give the crude amine as a grey oil. The material should be immediately used without purification for the next-step reaction since both prolonged storage at room temperature and flash chromatography would facilitate lactam formation.

To a solution of the above amine (˜9.15 mmol) in anhydrous dichloromethane (90 mL) cooled in an ice bath was added N,N-diisopropylethylamine (2.80 mL, 16.07 mmol) dropwise, followed by a solution of propargyl chloroformate (1.34 mL, 13.18 mmol) in dichloromethane (10 mL) dropwise over 20 min. The mixture was then stirred at room temperature for 12 h, and it was washed with sodium hydroxide solution (0.5 N, 20 mL) and brine (20 mL×2), dried (Na₂SO₄), evaporated, and flash chromatographed (EtOAc/hexanes, 1:3) to give compound 9 (2.47 g, 79% for two steps) as a colorless oil. R_{f}=0.28 (EtOAc/hexanes, 1:2); [α]_{D}²⁰ +3.9 (c 4.96, CH₂Cl₂); ¹H NMR (CDCl₃, 500 MHz) δ 5.16 (d, 1H, J=8.5 Hz), 5.12 (t, 1H, J=5.8 Hz), 4.62 (d, 2H, J=2.5 Hz), 4.26-4.21 (m, 1H), 3.14 (dt, 2H, J=6.5, 6.5 Hz), 2.45 (t, 1H, J=2.2 Hz), 1.80-1.73 (m, 1H), 1.64-1.57 (m, 1H), 1.53-1.44 (m, 2H), 1.40 (s, 9H), 1.36-1.30 (m, 2H); ¹³C NMR (CDCl₃, 125 MHz) δ 173.4, 155.7, 155.6, 80.0, 78.4, 74.7, 53.2, 52.4, 40.7, 32.3, 29.3, 28.4, 22.4; HRMS (ESI) calculated for C₁₆H₂₇N₂O₆ ([M+H]⁺) 343.1869, found 343.1871.

### 2.4—N-ε-Propargyloxycarbonyl-L-lysine hydrochloride (Compound/NAA #2)

To a solution of compound 9 (2.46 g, 7.18 mmol) in THF (30 mL) was added lithium hydroxide solution (0.5 M, 30.0 mL, 15.0 mmol), and the mixture was stirred at room temperature for 2 h. The mixture was diluted in water (20 mL) and extracted with ether (30 mL×2). The ether extracts were discarded, and the remaining aqueous solution was adjusted to pH 3 with hydrochloric acid (3N), with the concomitant formation of white precipitate. The suspension was extracted with ethyl acetate (60 mL×2), and the combined organic phases were washed once with brine (30 mL), dried (Na₂SO₄), and evaporated to give the crude carboxylic acid as a colorless oil, which was used without further purification.

The above crude acid (˜7.18 mmol) was dissolved in 1,4-dioxane (20 mL), and hydrogen chloride in 1,4-dioxane (4.0 M, 17.0 mL, 68.0 mmol) was added. The resulting white suspension was stirred at room temperature for 20 h, filtered, washed with dichloromethane, and dried to give NAA #2 (1.44 g, 76% for two steps) as a white solid. [α]_{D}²² +15.7 (c 1.26, 3N HCl); ¹H NMR (D₂O, 500 MHz) δ 4.67 (s, 2H), 4.04 (t, 1H, J=6.5 Hz), 3.16 (t, 2H, J=7.0 Hz), 2.90 (s, 1H), 2.04-1.89 (m, 2H), 1.57 (quintet, 2H, J=7.1 Hz), 1.53-1.38 (m, 2H); ¹³C NMR (D₂O, 125 MHz) δ 173.0, 158.3, 79.2, 76.2, 53.6, 53.3, 40.5, 30.0, 28.9, 22.0; HRMS (ESI) calculated for C₁₀H₁₇N₂O₄ ([M+H]⁺) 229.1188, found 229.1193. The characterization data matched well with that of the corresponding trifluoroacetic acid salt of NAA #2.

### 2.5—N-α-Boc-N-ε-cyclopentyloxycarbonyl-L-lysine methyl ester (Compound 10)

Compound 8 (2.23 g, 5.65 mmol) was converted into the corresponding amine by hydrogenolysis, which was then treated with cyclopentyl chloroformate (1.01 g, 6.79 mmol) according to the procedure for compound 9 to give compound 10 (1.12 g, 53% for two steps) as a white solid. R₁=0.35 (EtOAc/hexanes, 1:2); [α]_{D}²² +3.8 (c 1.20, CH₂Cl₂); ¹H NMR (CDCl₃, 500 MHz) δ5.13 (d, 1H, J=7.5 Hz), 5.06 (bs, 1H), 4.71 (bs, 1H), 4.28-4.24 (m, 1H), 3.72 (s, 3H), 3.14 (dt, 2H, J=6.3, 6.3 Hz), 1.82-1.76 (m, 3H), 1.66-1.64 (m, 5H), 1.55-1.44 (m, 4H), 1.42 (s, 9H), 1.39-1.29 (m, 2H); ¹³C NMR (CDCl₃, 125 MHz) δ 173.5, 156.8, 155.6, 80.0, 53.3, 52.4, 40.5, 32.94, 32.93, 32.5, 29.6, 28.5, 23.8, 22.6; HRMS (ESI) calculated for C₁₈H₃₃N₂O₆ ([M+H]⁺) 373.2339, found 373.2343.

### 2.6—N-ε-Cyclopentyloxycarbonyl-L-lysine hydrochloride (Compound 3)

According to the same procedure for NAA #2, compound 10 (1.06 g, 2.84 mmol) afforded NAA #3 (0.71 g, 85% for two steps) as a white solid. [α]_{D}²² +15.0 (c 1.14, 3N HCl) (lit.^{[5]} [α]_{D}²⁴ +13.0 (c 2.0, 80% acetic acid) for the corresponding free amino acid); ¹H NMR (D₂O, 500 MHz) δ 4.94 (m, 1H), 3.67 (t, 1H, J=6.2 Hz), 3.06 (t, 2H, J=7.0 Hz), 1.87-1.75 (m, 4H), 1.62 (m, 4H), 1.54-1.51 (m, 2H), 1.48 (quintet, 2H, J=7.0 Hz), 1.39-1.29 (m, 2H); ¹³C NMR (D₂O, pH=10, 125 MHz) δ 183.8, 159.5, 79.4, 56.5, 40.7, 34.7, 32.9, 29.5, 23.8, 22.8; FIRMS (ESI) calculated for C₁₂H₂₃N₂O_{4 ([M+H]}⁺⁾ 259.1658, found 259.1650.

### 2.7—5-(Propargyloxycarbonylamino)fluorescein (Compound 6)

To a solution of fluoresceinamine isomer I (also referred to herein as compound 11, 0.102 g, 0.29 mmol) (Aldrich) in anhydrous pyridine (1.0 mL) cooled in an ice bath was added propargyl chloroformate (58 μL, 0.59 mmol) dropwise, and the mixture was stirred at room temperature for 30 h. Ethyl acetate (50 mL) was then added, and the solution was washed with water (10 mL), saturated sodium bicarbonate (10 mL), hydrochloric acid (1N, 10 mL) and brine (10 mL), dried (Na₂SO₄), evaporated, and flash chromatographed (EtOAc/hexanes, 1:1) to give a yellow oil, which was dissolved in a minimal amount of ethyl acetate (˜0.5 mL) and precipitated with excessive hexanes (˜10 mL) to afford compound 6 (66 mg, 52%) as a bright orange solid. R₁=0.55 (EtOAc/hexanes, 2:1); ¹H NMR (CD₃OD, 500 MHz) δ 8.17 (s, 1H), 7.76 (dd, 1H, J=8.5, 2.0 Hz), 7.13 (d, 1H, J=9.0 Hz), 6.67 (d, 2H, J=2.5 Hz), 6.65 (d, 2H, J=9.0 Hz), 6.54 (dd, 2H, J=8.5, 2.5 Hz), 4.82 (d, 2H, J=2.5 Hz), 2.97 (t, 1H, J=2.5 Hz); ¹³C NMR (CD₃OD, 75 MHz) δ 171.6, 162.3, 155.0, 154.6, 142.3, 130.5, 126.7, 126.2, 114.9, 114.1, 111.9, 103.6, 79.2, 76.4, 53.6; HRMS (ESI) calculated for C₂₄H₁₆NO₇ ([M+H]⁺) 430.0927, found 430.0931.

### 3. DNA and Protein Sequences

### 3.1—DNA Sequences

pylT: ggaaacctgatcatgtagatcgaatggactctaaatccgttcagccgggttagattcccggggtttccgcca (SEQ ID NO: 1). The underlined sequence indicates the sequence corresponding to the anticodon of the tRNA.

pylT_{UUA}: ggaaacctgatcatgtagatcgaatggactttaaatccgttcagccgggttagattcccggggtttccgc ca (SEQ ID NO: 2). The underlined sequence indicates the sequence corresponding to the anticodon of the tRNA.

pylT_{UCA}: ggaaacctgatcatgtagatcgaatggacttcaaatccgttcagccgggttagattcccggggtttccgc ca (SEQ ID NO: 3). The underlined sequence indicates the sequence corresponding to the anticodon of the tRNA.

pylT_{UCUA}: ggaaacctgatcatgtagatcgaatggacttctaaatccgttcagccgggttagattcccggggtttcc gcca (SEQ ID NO: 4). The underlined sequence indicates the sequence corresponding to the anticodon of the tRNA.

GFP1TAG149TAA: atggcatagagtaaaggagaagaacttttcactggagttgtcccaattcttgttgaatta gatggtgatgttaatgggcacaaattttctgtcagtggagagggtgaaggtgatgcaacatacggaaaacttacccttaaatttattt gcactactggaaaactacctgttuccatggccaacacttgtcactactttctatatggtgttcaatgatttcccgttatccggatcacat gaaacggcatgactttttcaagagtgccatgcccgaaggttatgtacaggaacgcactatatctttcaaagatgacgggaactaca agacgcgtgctgaagtcaagtttgaaggtgatacccttgttaatcgtatcgagttaaaaggtattgattttaaagaagatggaaacat tctcggacacaaactcgagtacaactataactcacactaagtatacatcacggcagacaaacaaaagaatggaatcaaagctaa cttcaaaattcgccacaacattgaagatggatccgttcaactagcagaccattatcaacaaaatactccaattggcgatggccctgt ccttttaccagacaaccattacctgtcgacacaatctgccctttcgaaagatcccaacgaaaagcgtgaccacatggtccttcttga gtttgtaactgctgctgggattacacatggcatggatgaactctacaaagagctccatcaccatcaccatcactga (SEQ ID NO: 5). The underlined sequence indicates the sequence encoding the amber and ochre stop/blank codons corresponding to positions 1 and 149 of the protein, respectively.

### 3.2—Protein Sequence

GFP1TAG149TAA: maxskgeelftgvvpilveldgdvnghkfsysgegegdatygkltlkficttgklp vpwptlvttfsygvqcfsrypdhmkrhdffksampegyvqertisfkddgnyktraevkfegdtlvnrielkgidfkedgni lghkleynynshx*vyitadkqkngikanfkirhniedgsvqladhyqqntpigdgpvllpdnhylstqsalskdpnekrd hmvllefvtaagithgmdelykelhhhhhh (SEQ ID NO: 6). The x and x* indicate the incorporated noricanonical amino acids at positions 1 and 149 of the GFP_{UV} protein.

### 4. Construction of Plasmids

All the plasmid structures have been confirmed by DNA sequencing. All oligonucleotide primers were purchased from Integrated DNA Technologies, Inc.

### 4.1—Construction of pAcKRS-pylT-GFP1Ochre, pAcKRS-pylT-GFP1Opal, and pAcKRS-pylT-GFP1four Base

pAcKRS-pylT-GFP1Opal contains genes encoding AcKRS, pylT_{UCA} and GFP_{UV} with an opal mutation corresponding to position 149. This plasmid was derived from pAcKRS-pylT-GFP1Amber, described in Y. Huang, et al., Bioorg. Med. Chem. Len., 20:878 (2010). The standard QuikChange site-directed mutagenesis was used to mutate the anticodon of pylT in pAcKRS-pylT-GFP1Amber to TCA first. The resulting plasmid then underwent the second QuikChange site-directed mutagenesis to mutate the TAG codon corresponding to position 149 of GFP_{UV} to the TGA codon.

Similarly, two sequential QuikChange site-directed mutagenesis experiments were carried out to mutate the anticodon of pylT and the amber mutation of GFP_{UV} in pAcKRS-pylT-GFP1Amber to form pAcKRS-pylT-GFP1Ochre and pAcKRS-pylT-GFP1four base.

### 4.2—Construction of pBK-PylRS

The plasmid pBK-PylRS was derived from pBK-JYRS, which is described in Wang, L., et al., Science, 292:498 (2001). The gene of Methanosarcina mazei PylRS was PCR amplified from Methanosarcina mazei genomic DNA which was purchased from ATCC. Two restriction sites, NdeI at the 5′ end and PstI at the 3′ end, were introduced in the PCR product which was subsequently digested and cloned into pBK-JYRS to replace JYRS.

### 4.3—Construction of pAcKRS-pylT-GFP1TAG149TAA

The plasmid pAcKRS-pylT-GFP1TAG149TAA was derived from pAcKRS-pylT-GFP1Ochre that contains pylT_{UUA} and GFP_{UV} with an ochre mutation corresponding to position 149 (GFP1Ochre). GFP1TAG149TAA (SEQ ID NO: 5) was first generated by

PCR amplification of GFP1Ochre with primers that add one amber mutation at the first codon, two additional codons (ATGGCA) in front of the first codon, and TGA stop codon at the end of the gene. This gene was then cloned into the pAcKRS-pylT-GFP1Ochre between NdeI and KpnI sites to afford pAcKRS-pylT-GFP1TAG149TAA. In the final plasmid, GFP1Ochre was replaced by GFPITAG149TAA.

### 4.4—Construction of pPylRS-pylT-GFP1TAG149TAA

The plasmid pPylRS-pylT-GFP1TAG149TAA was derived from pAcKRS-pylT-GFP1AG149TAA. The PylRS gene flanked by the constitutive glutamine promoter at the 5′ end and the glutamine terminator at the 3′ end was PCR amplified from pBK-PylRS. Two restriction sites, ClaI at the 5′ end and HindIII at the 3′ end, were introduced in the synthesized DNA which was subsequently digested by these two enzymes and cloned into pAcKRS-pylT-GFP1AG149TAA to replace AcKRS.

### 4.5—Construction of pEVOL-sTyrRS

The plasmid pEVOL-sTyrRS was derived from pEVOL-AzFRS, which is described in Young, T. S., et al., J. Mol. Biol., 395:361 (2010). sTyrRS was PCR amplified from pBK-sTyrRS, which is described in Liu, C. C. and Schultz, P. G., Nat. Biotech., 24:1436 (2006). It was cloned into NdeI and PstI sites of pEVOL-AzFRS to replace the first AzFRS copy. After confirmation by DNA sequencing, the second sTyrRS copy was cloned into BglII and PstI sites to replace the second copy of AzFRS to afford pEVOL-sTyrRS.

### 5. Orthogonality Test

To test the interaction between PylRS and MjtRNA(Tyr/CUA), two plasmids, pBK-pylRS and pREP, were used to transform E. coli Top10 cells. pREP contains genes encoding MjtRNA(Tyr/CUA) and a chloramphenicol acetyltransferase with an amber mutation at position 112. The transformed cells were grown on LB plates containing 25 μg/mL kanamycin and 12 μg/mL tetracycline. Five single colonies from the kanamycin/tetracycline plate was transferred onto a LB plate containing 25 μg/mL kanamycin, 12 μg/mL tetracycline, 34 μg/mL chloramphenicol, and 1 mM N^{ε}-Boc-L-lysine. None of the colonies were viable. A similar experiment was carried out to test the interaction between MjTyrRS and pylT. pBK-JYRS that contains wild type MjTyrRS and pREP in which MjtRNA_{CUA}^{Tyr} was replaced with pylT were used to transform E. coli Top10 cells. The growth of the transformed cells on a LB plate containing 25 μg/mL kanamycin, 12 μg/mL tetracycline and 34 μg/mL chloramphenicol did not lead to any viable clones.

### 6. Suppression Test

E. coli BL21 cells transformed with pAcKRS-pylT-GFP1Amber were grown in 5 ml of LB medium at 37° C. overnight, and the culture was subsequently inoculated into 50 mL of LB supplemented with 100 μg/mL ampicillin. The expression of GFP_{UV} was then induced with the addition of 500 μg/mL IPTG and 1 mM N-ε-acetyl-L-lysine (AcK) when the OD₆₀₀ reached 0.6. After induction, the culture was incubated at 37° C. for 6 h. Cells were then harvested by centrifugation at 4500 r.p.m. for 20 min at 4° C. and resuspended in 20 mL of lysis buffer (50 mM HEPES, 500 mM NaCl, 10 mM DTT, 10% glycerol, 0.1% Triton X-100, 5 mM imidazole, and 1 μg/mL lysozyme, pH 7.4). The resuspended cells were sonicated in ice water bath three times (4 min each, 5 min interval to cool the suspension below 10° C. before the next run) and the lysate was clarified by centrifugation at 10200 r.p.m. for 60 min at 4° C. More lysis buffer was added to the supernatant to make a final volume of 50 mL. The expression of GFP_{UV} from cells transformed with pAcKRS-pylT-GFP1Amber, pAcKRS-pylT-GFP1Opal, pAcKRS-pylT-GFP1Ochre and pAcKRS-pylT-GFP1four base were carried out following exactly the same procedures. Fluorescence spectroscopic studies of the final clarified GFP_{UV} solution were performed on a Cary Eclipse fluorometer. The slit width was 5 nm for both excitation and emission. The fluorescence of each sample was excited at 385 nm and then measured at 505 nm.

### 7. Protein Expression and Purification

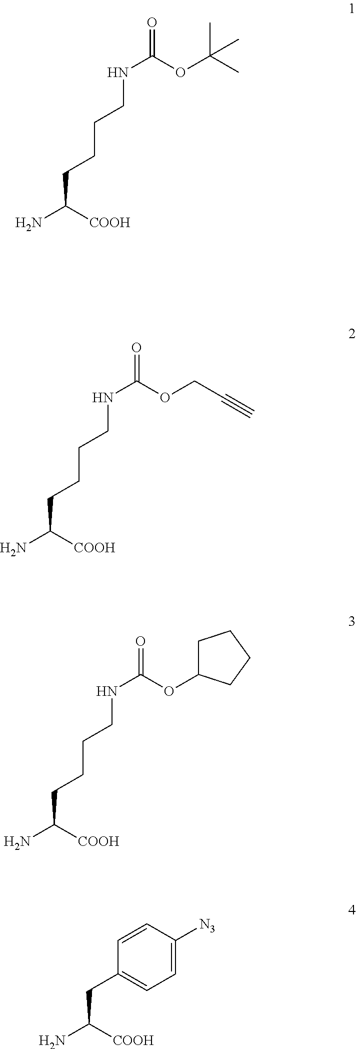 [SMILES: CC(C)(C)OC(=O)NCCCC[C@H](N)C(=O)O ; C#CCOC(=O)NCCCC[C@H](N)C(=O)O ; N[C@@H](CCCCNC(=O)OC1CCCC1)C(=O)O ; [N-]=[N+]=Nc1ccc(C[C@H](N)C(=O)O)cc1]

### 7.1—Expression and Purification of GFP_{UV}(1+4), GFP_{UV}(2+4), GFP_{UV}(3+4) from Cells Transformed with pPylRS-pylT-GFP1TAG149TAA and PEVOL-AzFRS

E. coli BL21 cells transformed with pPylRS-pylT-GFP1TAG149TAA together with PEVOL-AzFRS were grown in 2YT medium (50 mL) at 37° C. overnight. The culture was inoculated into 2YT medium (150 mL) containing 100 μg/mL ampicillin and 25 μg/mL kanamycin and then induced with the addition of 500 μg/mL IPTG and 0.02% arabinose when the OD₆₀₀ reached 0.6. Compound/NAA #4 with either one of compounds/NAA #s 1, 2, and 3 were added so that there was 1 mM each of the two noncanonical amino acids. The culture was then incubated at 37° C. for 8 h, and cells were harvested and lysed as described above. The supernatants were loaded onto an AKTApurifier UPC 10 FPLC system (GE Healthcare) equipped with a Ni-NTA agarose (Qiagen Inc.) column. The column was washed with 5× bed volumes of buffer A (50 mM HEPES, 300 mM NaCl and 5 mM imidazole, pH 7.5) and then eluted by running a gradient from 100% buffer A to 100% buffer B (50 mM HEPES, 300 mM NaCl and 50 mM imidazole, pH 7.5) in 10× bed volumes. The eluted proteins were concentrated by Amicon Ultra-4 Centrifugal Filter Units (Millipore, NMWL 10 KDa) and analyzed by 12% SDS-PAGE. The protein concentrations were determined by BCA assay kits purchased from Pierce Inc.

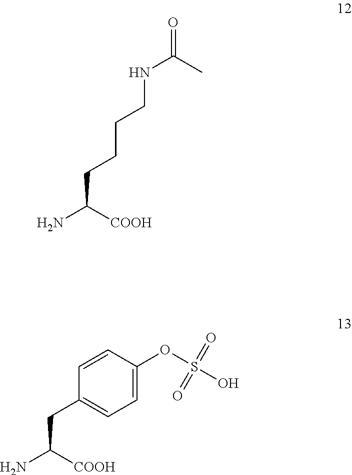 [SMILES: CC(=O)NCCCC[C@H](N)C(=O)O ; N[C@@H](Cc1ccc(OS(=O)(=O)O)cc1)C(=O)O]

### 7.2—Expression and Purification of GFP_{UV}(4+12) and GFP_{UV}(2+13)

GFP_{UV} incorporated with compound 4 at position 1 and compound 12 at position 149 (GFP_{UV}(4+12); 12: AcK) and GFP_{UV} incorporated with compound 13 at position 1 and compound 2 at position 149 (GFP_{UV}(2+13); 13: sTyr) were expressed by using two other sets of plasmids. GFP_{UV}(4+12) was expressed in E. coli BL21 cells transformed with pAcKRS-pylT-GFP1TAG149TAA together with PEVOL-AzFRS. The transformed cells were grown in 2YT medium (50 mL) at 37° C. overnight, and the culture was subsequently inoculated into 150 ml of 2YT containing 100 μg/mL ampicillin and 25 μg/mL kanamycin. The GFP_{UV} expression was induced with the addition of 500 μg/mL IPTG, 0.02% arabinose, 1 mM 4, and 5 mM 12 when the OD₆₀₀ reached 0.6. The culture was then let grown at 37° C.; or 8 h. The cells were harvested and the proteins were purified using the same procedures as described above. GFP_{UV}(2+13) was similarly expressed in E. coli BL21 cells transformed with pPylRS-pylT-GFP1TAG149TAA together with PEVOL-sTyrRS in the presence of 1 mM 2 and 2 mM 13.

### 7.3—Expression of Wild-type GFP_{UV}

A pREP plasmid, as described in Santoro, S. W., et al., Nat. Biotech. 20:1044 (2002), that contains wild-type GFP_{UV} gene under control of the T7 promoter was transformed into BL21 cells. The transformed cells were grown in 5 mL of LB medium supplemented with 12 μg/mL tetracycline at 37° C. overnight, and the culture was inoculated into 200 mL of LB medium containing 12 μg/mL tetracycline and let grown at 37° C. for 8 h. Cells were then harvested by centrifugation at 4500 r.p.m. for 20 min at 4° C. and resuspended in 20 mL of lysis buffer (50 mM HEPES, 500 mM NaCl, 10 mM DTT, 10% glycerol, 0.1% Triton X-100, 5 mM imidazole, and 1 μg/mL: lysozyme, pH 7.4). The resuspended cells were sonicated in ice water bath three times (4 min each, 5 min interval to cool the suspension below 10° C. before the next run) and the lysate was clarified by centrifugation at 10200 r.p.m. for 60 min at 4° C. The supernatant that contained high concentration of GFP was then fractionated by the addition of 70% ammonium sulfate to effect precipitation. The precipitate was then redissolved in 100 mM sodium phosphate buffer (pH 7.6) to make a 4 mg/mL solution, which was directly used for protein labeling assay.

### 8. Mass Spectrometry Analysis

An Agilent (Santa Clara, Calif.) 1200 capillary HPLC system was interfaced to an API QSTAR Pulsar Hybrid QTOF mass spectrometer (Applied Biosystems/MDS Sciex, Framingham, Mass.) equipped with an electrospray ionization (ESI) source. Liquid chromatography (LC) separation was achieved using a Phenomenex Jupiter C4 microbore column (150×0.50 mm, 300 Å) (Torrance, Calif.) at a flow rate of 10 μL/min. The proteins were eluted using a gradient of (A) 0.1% formic acid versus (B) 0.1% formic acid in acetonitrile. The gradient timetable for B was as follows: starting at 2% for 5 min, 2-30% in 3 min, 30-60% in 44 min, 60-95% in 8 min, followed by holding the gradient at 95% for 5 min, for a total run time of 65 min. The MS data were acquired in positive ion mode (500-1800 Da) using spray voltage of +4900 V. BioAnalyst software (Applied Biosystems) was used for spectral deconvolution. A mass range of m/z 500-1800 was used for deconvolution and the output range was 10000-50000 Da using a step mass of 0.1 Da and a S/N threshold of 20.

### 9. Protein Labeling

GFP_{UV}(2+4) in 100 mM sodium phosphate buffer (pH 7.6) was concentrated to 4 mg/mL. To an aqueous sodium ascorbate solution (100 mM, 6 μL, 0.6 μmol) was added aqueous sulfonated bathophenanthroline sodium salt solution (100 mM, 8 μL, 0.8 μmol) followed by copper sulfate (100 mM, 5 μL, 0.5 μmol), and the mixture was incubated at room temperature for 5 min. Compound 5 (10 mM in CH₂Cl₂, 20 μL, 0.2 μmol) or compound 6 (10 mM in CH₂Cl₂, 20 μL, 0.2 μmol) was deposited in a 0.5 mL centrifuge tube and the solvent was evaporated by gentle air blowing. GFP_{UV}(2+4) (4 mg/mL, 10 μL, 1.4 nmol) was then added, followed by the addition of the above catalyst (2 μL each). The mixture was reacted at 4° C. for 2 h to give a clear solution which was then directly loaded onto 12% SDS-PAGE for analysis.

### Example 2

This Example describes the development of a catalyst-free and site-specific one-pot dual labeling of a protein directed by two genetically incorporated noncanonical amino acids

Introduction

Förster resonance energy transfer (FRET) between a pair of donor and acceptor dyes is an invaluable tool to study dynamic protein conformational changes such as conformation rearrangement and folding/unfolding. The efficiency of energy transfer that is dependent on the distance between the two dyes not only represents the conformational distributions but also reflects their change upon time. Two methods are usually applied to achieve dual labeling of a protein with a FRET pair. One is to genetically fuse two green fluorescent protein (GFP) variants at the N- and C-termini of a protein. The other is to modify two cysteine residues in a protein with a small-molecule FRET pair. The GFP labeling approach has important advantages such as high labeling specificity and simplicity. However, this approach has its intrinsic limitations. Labeling a protein with two GFP variants is, in general, restricted at the two termini. The size of GFP (˜27 kDa) is also large enough to potentially interfere with the structures and functions of proteins to which they are fused. The cysteine labeling approach resolves issues such as site and size restrictions associated with the GFP labeling approach. This approach also has advantages such as the flexibility in choosing small molecule fluorophores and achieving labeling at the single residue level. The straightforward labeling reactions and the commercial availability of many thiol-reactive dyes have made the cysteine labeling approach a favourable choice especially for the single-molecule FRET analysis. However, the cysteine labeling approach also has its drawbacks. It requires mutating all nontargeted cysteine residues, and is therefore not applicable for proteins in which cysteine residues serve critical structural and/or functional roles. In addition, dual labeling of two cysteine residues, in general, leads to heterogeneously labeled proteins due to the lack of selectivity between two cysteine residues.

To achieve site selective labeling of a protein with a small-molecule FRET pair, several other methods have been developed, including total chemical synthesis of a modified protein, specific modification of a protein at its N-terminus and a cysteine residue, the use of the protein-protein interaction to protect a cysteine to achieve sequential labeling of two cysteine residues, and the combination of chemical and enzymatic modifications. These methods either work for only specific proteins or are complicated so that their general applications are difficult. An alternative dual labeling approach incorporates a cysteine residue and a noncanonical amino acid (NAA), p-acetyl-L-phenylalanine coded by an amber codon, which are used to site-selectively label a protein. While elegant, this approach still suffers limitations such as the requirement to mutate non-targeted cysteine residues and is not applicable to most mammalian proteins.

An ideal dual labeling approach that resolves the issues related to methods discussed above is to install two different bio-orthogonal and chemically reactive groups into a protein followed by their selective reactions with corresponding dyes. One way to achieve this goal is to incorporate two different NNAs into a target protein in a site-selective manner. As described above in Example 1, the present disclosure provides a novel method for the facile genetic incorporation of two different NAAs into one protein. The method relies on the suppression of two stop codons, namely the amber UAG codon and the ochre UAA codon, which is achieved by genetically encoding two orthogonal pairs, an evolved M. jannaschii tyrosyl-tRNA synthetase (MjTyrRS)-tRNA(Tyr/CUA) pair and a wild type or evolved pyrrolysyl-tRNA synthetase (PylRS)-tRNA(Pyr/UUA) pair. As described herein, this double NAA incorporation method was successfully applied to genetically install two bioorthogonal functional groups into one protein, allowing the catalyst-free and site-specific one-pot labeling of the protein with a FRET pair.

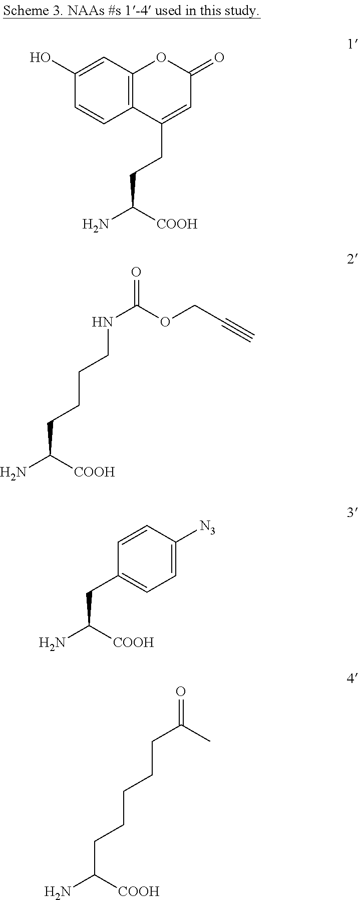 [SMILES: N[C@@H](CCc1cc(=O)oc2cc(O)ccc12)C(=O)O ; C#CCOC(=O)NCCCC[C@H](N)C(=O)O ; [N-]=[N+]=Nc1ccc(C[C@H](N)C(=O)O)cc1 ; CC(=O)CCCCCC(N)C(=O)O]

It is noted that the compounds referred to in this Example with Arabic numerals are identified throughout with a “′” to distinguish them from any potentially different compounds referred to above in Example 1, which also uses arabic number designations. As a first step, one fluorescent NAA, L-(7-hydroxycoumarin-4-yl)ethylglycine (NAA #1′ in Scheme 3, also referred to in this Example as 1′) and N^{ε}-propargyloxycarbonyl-L-lysine (NAA #2′ in Scheme 3, also referred to in this Example as 2′) was incorporated into glutamine binding protein (QBP) at positions 3 and 141, respectively, followed by the copper(I)-catalyzed azide-alkyne Huisgen cycloaddition (CuAAC) click reaction on NAA #2′ to generate a dual-labeled QBP. Because NAA #1′ is blue fluorescent itself, its incorporation into QBP at an amber mutation site using an evolved MjTyRS (CouRS)-tRNA(Tyr/CUA) pair will lead to quantitative blue fluorescent labeling of QBP. QBP incorporated with both NAA #1′ and NAA #2′ will only require one following labeling reaction on NAA #2′ to install a FRET pair, therefore simplifying the FRET installation procedure. To incorporate NAA #1′ and NAA #2′ into QBP, the QBP gene with an amber mutation at its amino acid position 3 and an ochre mutation at its amino acid position 141 was generated. A nucleic acid sequence for the QBP with an amber mutation and ochre mutation encoded corresponding to positions 3 and 141, respectively, is set forth as SEQ ID NO: 16. The mutated QBP gene was cloned into plasmid pETtrio-pylT-PylRS-MCS, which was derived from pPylRS-pylT-GFP1TAG149TA and carries genes coding the PylRS-tRNA(Pyl/UUA) pair, to afford pETtrio-pylT-PylRSQBP3TAG141TAA. This plasmid was coupled together with pEVOL-CouRS that carries genes coding the CouRS-tRNA(Tyr/CUA) pair to cotransform E. coli BL21 cells. Unfortunately, growing the transformed cells in the presence of 1 mM NAA #1′ and 1 mM NAA#2′ resulted in only negligible QBP expression. This low QBP expression level is likely due to the low incorporation efficiency of NAA #1′ mediated by CouRS.

Next, other NAAs were assessed for the capacity to be coupled together with NAA #2′ to be incorporated into QBP for a FRET pair installation. Because p-acetyl-L-phenylalanine needs to react with hydroxylamine dyes at pH<4, a condition that denatures many proteins, p-azido-L-phenylalanine (NAA #3′ in Scheme 3, also referred to in this Example as 3′) was used because it can undergo the CuAAC reaction. To incorporate NAA #2′ and NAA #3′ into QBP, pETtrio-pylTPylRS-QBP3TAG141TAA and pEVOL-AzFRS were used to cotransform E. coli BL21 cells. Plasmid pEVOL-AzFRS carries genes coding an evolved NAA #3′-specific MjTyRS (AzFRS) and tRNA(Tyr/CUA). The transformed cells were then used to express QBP with NAA #3′ and NAA #2′ incorporated at positions 3 and 141, respectively, (QBP(3′+2′)). QBP was overexpressed when 1 mM NAA #2′ and 1 mM NAA #3′ were provided in the growth medium. The expression yield in 2YT medium was 14 mg/L. Providing one NAA or no NAA in the medium only gave negligible QBP expression (not shown).

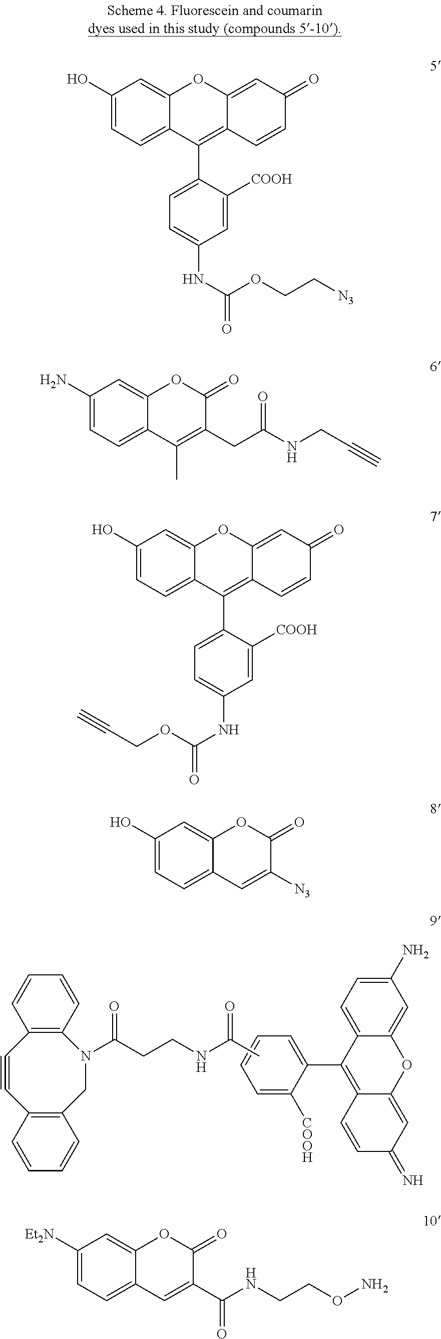

Since both NAA #2′ and NAA #3′ can undergo the CuAAC reaction, labeling of QBP(3′+2′) was carried out in two separate steps to avoid cross-reactions of dyes. The initial trials of labeling 3′ of QBP(3′+2′) with a coumarin alkyne (compound 6′ in Scheme 4, also referred to in this Example as 6′) in the presence of a Cu(I):tris[(1-benzyl-1H-1,2,3-triazol-4-yl)methyl]amine (TBTA) complex and 5 mM ascorbate met problems due to several reasons. QBP(3′+2′) has a 6×His tag at its C-terminus for its affinity purification using Ni-NTA resins. Imidazole that was used to elute QBP(3′+2′) from Ni-NTA resins adversely influenced the CuAAC labeling process. Directly performing the CuAAC reaction between QBP(3′+2′) and 6′ in a buffer containing 250 mM imidazole gave poor labeling efficiency. A three-hour reaction only led to an insignificant amount of labeled QBP(3′+2′) as revealed by the electrospray ionization mass spectrometry (ESI-MS) analysis of the final reaction mixture. Thoroughly dialysing imidazole from QBP(3′+2′) did increase the labeling efficiency but not to a great extent. According to Hong, V., et al., Angew Chem Int Ed Engl, 48:9879-9883 (2009), the 6×His tag of QBP(3′+2′) could potentially chelate Cu(I) and thus reduce the effective Cu(I) concentration for catalysis. Thus, to counteract the chelating effect of the 6×His tag, 1 mM Ni²⁺ was later provided in the reaction mixture. Performing the reaction in the optimized labeling condition that had 0.1 mM Cu(I):TBTA complex, 0.4 mM additional TBTA, 5 mM ascorbate, and 1 mM NiCl₂ for three hours afforded nearly quantitatively labeling of QBP(3′+2′) with compounds 6′ to form QBP(3′+2′)-6′, as confirmed by the ESI-MS analysis of the final product (not shown). The ratio of dye to protein during the reaction was 50 to 1. A similar reaction was also carried out to label QBP(3′+2′) with 5′ to form QBP(3′+2′)-5′ (not shown). To generate a final FRET pair in QBP, QBP(3′+2′)-6′ was purified using Ni-NTA resins to remove the unreacted 6′ and then reacted with 5′ in the same optimized CuAAC conditions for 3 h to form QBP(3′+2′)-6′-5′. The ESI-MS analysis of the final product suggested close to 80% conversion from QBP(3′+2′)-6′ to QBP(3′+2′)-6′-5′ (not shown). QBP(3′+2′)-6′-5′ was further purified using Ni-NTA resins to remove the unreacted 5′. To demonstrate the application of this dual labeled protein, it was used to undergo protein unfolding analysis using guanidinium chloride (GndCl). When excited at 350 nm and with increasing GndCl concentration, QBP(3′+2′)-6′-5′ displayed fluorescent emission increase from 6′ at 430 nm and fluorescent emission decrease from 5′ at 520 nm, suggesting the distance increase between the two dyes during the protein unfolding process.

Although two optimized consecutive CuAAC reactions were used to label QBP(3′+2′) with 6′ and 5′ to achieve highly efficient dual labeling, there are downsides related to the CuAAC reaction on proteins. During labeling QBP(3′+2′) with 5′, it was noted that a significant amount of protein aggregated. This aggregation process was time dependent. While 47% of QBP(3′+2′) was recovered after labeling with 5′ for 1 h, only 12% was recovered after a 5 h reaction. Because the two labeling reactions have to be performed sequentially for 3 h each and the protein has to be purified by Ni-NTA resins after each round to remove residual dyes, the protein recovery rate is low. The finally obtained product QBP(3′+2′)-6′-5′ only accounted for 9% of the original QBP(3′+2′). This protein aggregation and low recovery problem poses a challenge to using other, less reactive dyes to label QBP(3′+2′). The dyes indicated as compounds 7′ and ′8, as shown in Scheme 4, were also used to achieve a dual-labeled QBP(3′+2′). Although the reaction between QBP(3′+2′) with 8′ under the optimized CuAAC conditions could be finished in 3 h, the reaction between QBP(′3+2′) and 7′ was less than 50% completed after 6 h, a time at which much protein aggregated. Besides the protein aggregation issue, another problem associated with the CuAAC reaction is protein oxidation. It has been demonstrated in the previous literature that Cu(I) can promote the formation of reactive oxygen species, leading to protein oxidation. As confirmed by the ESI-MS analysis, the QBP(3′+2′)-5′-6′ produced here did show a large unexpected peak at 26,818 Da, which matches the molecular weight of the desired product with one additional oxygen atom. The final product also has a messy ESI-MS spectrum that might result from undesired modifications of the protein during the labeling process. One option is to provide some reactive oxidative species scavengers and purge the reaction mixture with Argon followed by an anaerobic labeling reaction, which might serve to alleviate side reactions. However, these additional treatments can complicate the labeling process complicated and hard to perform.

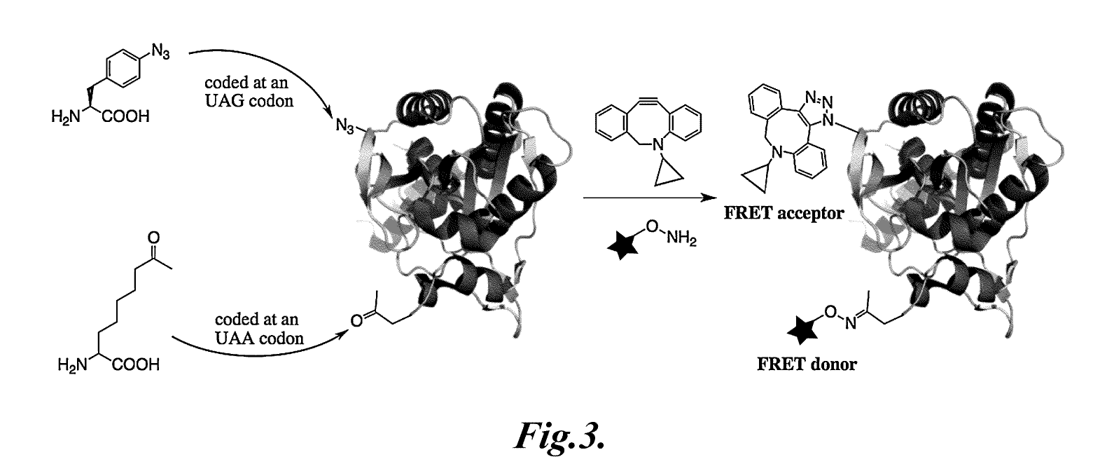

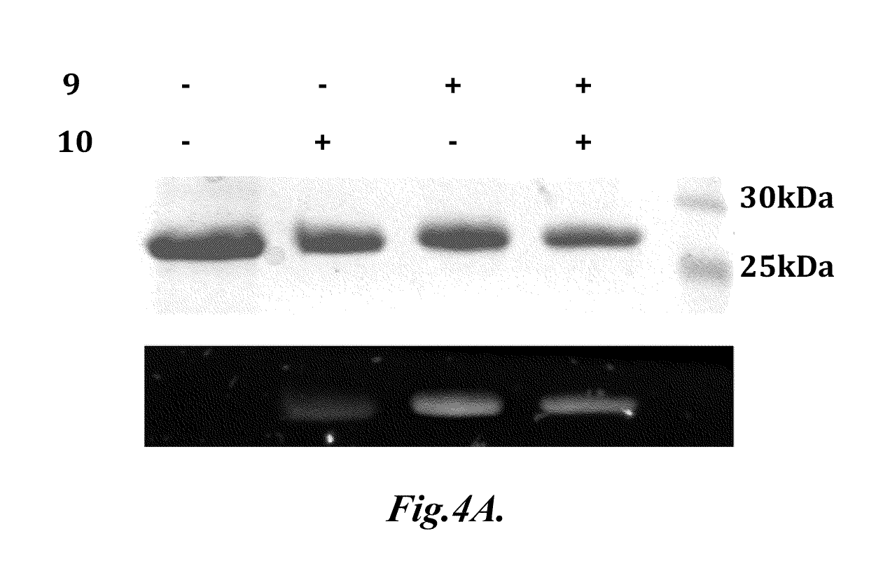

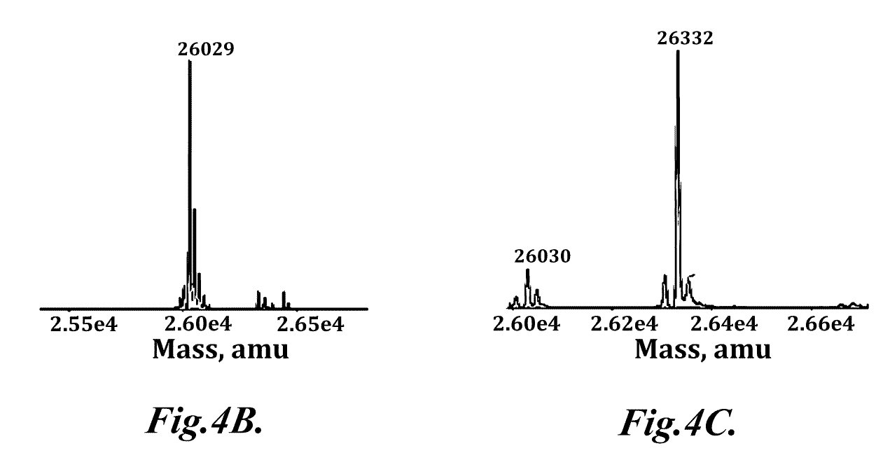

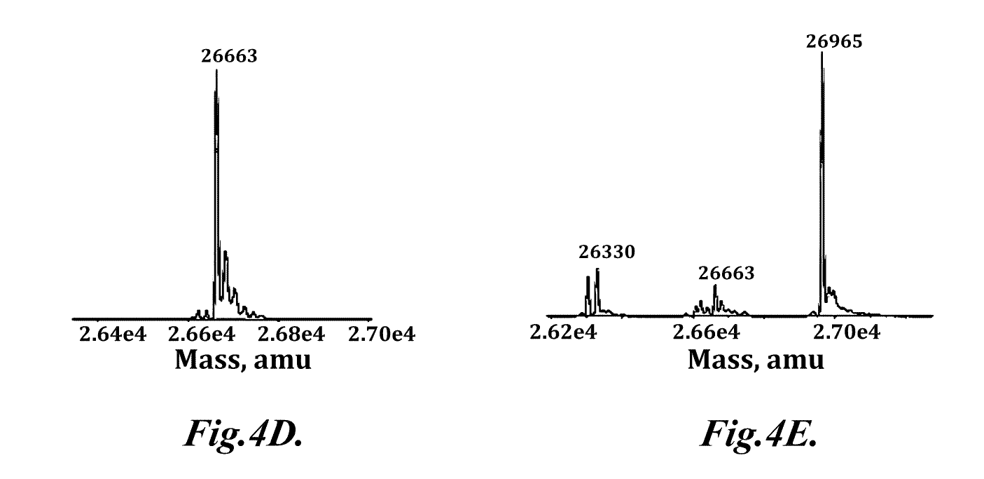

A more ideal dual labeling method that resolves the above issues associated with two consecutive CuAAC reactions is to run two labeling reactions in “one pot” and in a catalyst-free fashion. An exemplary embodiment of this one pot method is illustrated in FIG. 3, wherein one azide-containing noncanonical amino acid is incorporated at an amber mutation site and one different keto-containing noncanonical amino acid is incorporated at an ochre mutation site, followed by their orthogonal reactions with a cyclooctyne-containing dye and a hydroxylamine-containing dye, respectively. To run two labeling reactions in one pot, they not only need to be bioorthogonal (relative to the cell's endogenous translation machinery) but must be orthogonal to each other. Two reactions that meet this requirement and also are catalyst free are the azide cyclooctyne click reaction and the oxime formation reaction. Although the rate of the azide cyclooctyne click reaction is slower than the CuAAC reaction, presumably there is no protein aggregation due to the lack of metal catalysts so that the reaction time could be prolonged to achieve high labeling efficiency. It was previously showed that a keto-containing NAA, NAA #4′ in Scheme 3 above, could be genetically incorporated into proteins at amber mutation sites using an evolved M. barkeri PylRS (MbAcKRS1)-tRNA(Pyl/CUA) pair. Since the labeling of 4′ with a hydroxylamine dye could be achieved at a physiological pH, the incorporation of 3′ and 4′ into a protein will enable labeling the protein with a hydroxylamine dye and a cyclooctyne dye in a one-pot and catalyst-free fashion. Because MbAcKRS1 has a rather low efficiency for the incorporation of 4′, the more efficient MmAcKRS that was evolved from M. mazei PylRS and has mutations L301M/Y306L/L309A/C348F. This gene was used to replace PylRS in pETtrio-pylTPlyRS-QBP3TAG141TAA to afford pETtrio-pylT-MmAcKRSQBP3TAG141TAA. Transforming E. coli BL21 cells with pEVOLAzFRS and pETtrio-pylT-MmAcKRS-QBP3TAG141TAA and growing the transformed cells in the presence of 1 mM NAA #3′ and 2 mM NAA #4′ led to overexpression of QBP with 3′ and 4′ incorporated at positions 3 and 141, respectively (QBP(3′+4′)). The protein expression yield was 13 mg/L in 2YT medium. Providing only one NAA or no NAA in the medium gave a negligible QBP expression level (not shown). With QBP(3′+4′) in hand, a one-pot and catalyst-free dual label process of QBP(3′+4′) was performed using dyes 9′ and 10′, indicated as compounds 9′ and 10′ as shown in Scheme 4. Overnight incubation at pH 6.4 resulted in close to full conversion of the starting protein to the desired dual-labeled QBP(3′+4′) (QBP(3′+4′)-9′-10′), as indicated by the ESI-MS spectrum of the final product (FIG. 4E). Moreover, after affinity purification using Ni-NTA resins to remove unreacted dyes, the finally obtained QBP(3′+4′)-9′-10′ accounted for 83% of the original QBP(3′+4′). In comparison to QBP(3′+2′)-6′-5′ shown FIG. 4E, QBP(3′+4′)-9′-10′ displayed a much cleaner ESI-MS spectrum, suggesting that no additional modification took place during the labeling process. Two separate reactions to label QBP(3′+4′) with 9′ and 10′ individually were also carried out. FIG. 4A illustrates labeling of QBP(3′+4′) with dye compounds 9′ and 10′, separately and together. The top panel illustrates Coomassie blue stained proteins in a SDS-PAGE gel, whereas the bottom panel illustrates fluorescent imaging of the same gel under irradiation of 365 nm UV light with the image shows real colors captured by a regular camera. In color, the bottom panel shows lanes with no band, blue band, green band, and blue/green band, from left to right. Each reaction led to close to quantitative labeling (FIGS. 4C&D). With a much simpler labeling procedure and a much better protein recovery yield, this one-pot catalyst-free dual-labeling method is drastically superior to the dual labeling achieved by two consecutive CuAAC reactions. Of note is the pH dependence of the oxime formation reaction. The labeling of QBP(3′+4′) with 10′ at pH 8.1 resulted in a negligible level of labeling after overnight incubation.

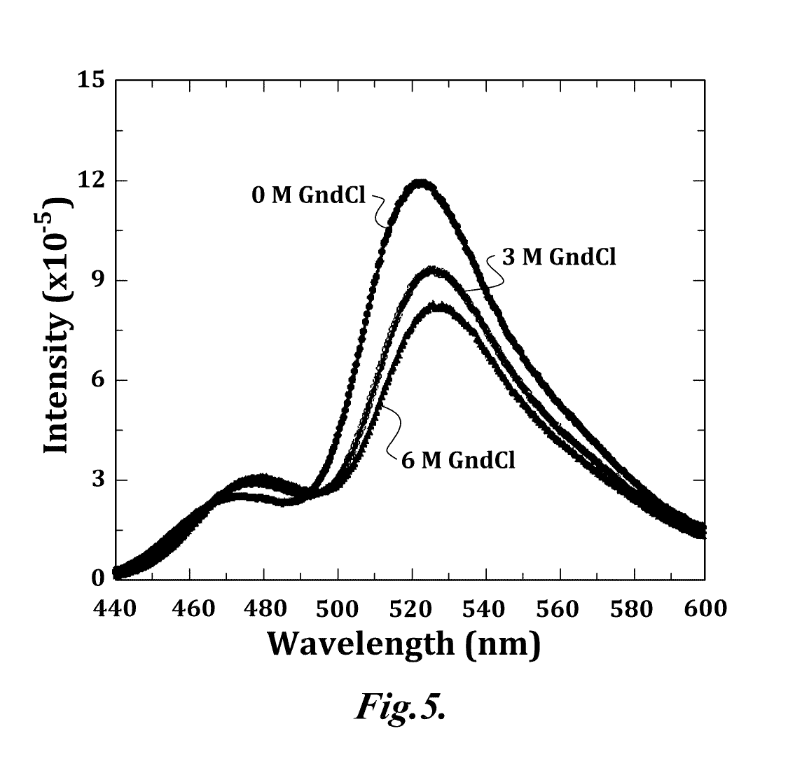

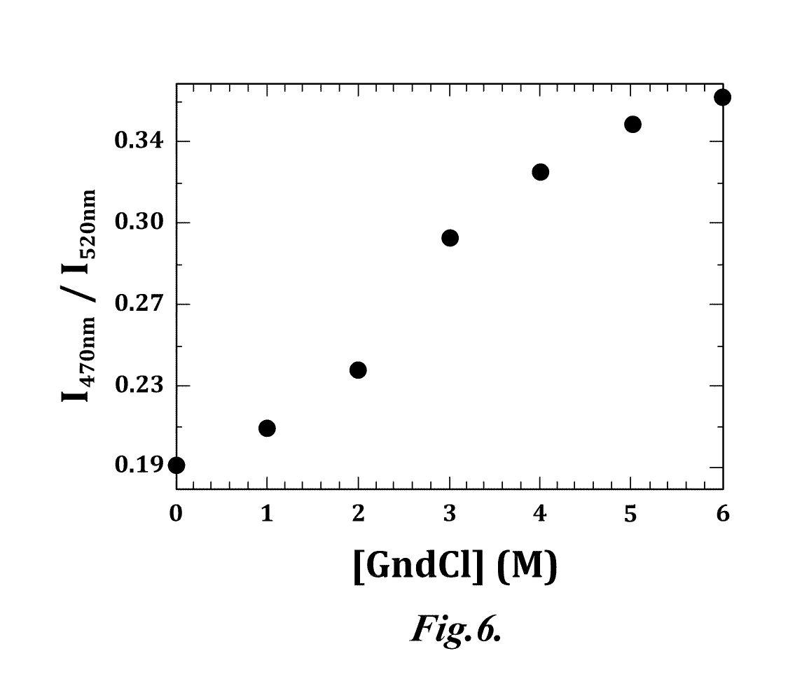

Additionally, QBP(3′+4′)-9′-10′ was used to carry out protein unfolding analysis. When excited at 430 nm and with increasing GndCl concentration, QBP(3′+4′)-9′-10′ displayed fluorescent emission increase from 10′ at 470 nm and fluorescent emission decrease from 9′ at 520 nm (FIG. 5), suggesting the distance increase between the two dyes during the unfolding process. FIG. 6 illustrates the smooth unfolding pattern determined by the change of I₄₇₀ₙₘ/I₅₂₀ₙₘ.

In summary, this Example describes the development of an optimal protein dual-labeling method that can be carried out in a “one-pot” and catalyst-free fashion. The two reactions for the dual labeling are both biocompatible. No treatment of proteins to avoid non-specific modifications with amino acid side chains such as cysteine thiols is necessary. The two reactions are orthogonal to each other and are directed by two genetically incorporated NAAs, assuring the labeling specificity and selectivity and at the same time keeping other residues intact. The two labeling reactions are also highly efficient, leading to almost quantitative labeling after an overnight incubation. The recovery yield of the finally labeled protein is also excellent. This simple and straightforward protein dual-labeling method resolves limitations associated with other current dual-labeling strategies and can be easily adopted by other research groups. Its potential applications range from single molecule FRET studies of protein dynamics, protein folding/unfolding investigations, and biosensor development.

Methods and Materials

Except for dye 9′, which was purchased from Click Chemistry Tools, all NAAs and dyes were synthesized. The synthesis of these compounds, plasmid constructions, protein expression and purification, protein labeling, and FRET analysis of QBP unfolding are described in more detail below.

### 1. General Experimental Description

All reactions involving moisture sensitive reagents were conducted in oven-dried glassware under an argon atmosphere. Anhydrous solvents were obtained through standard laboratory protocols. Analytical thin-layer chromatography (TLC) was performed on Whatman SiO₂ 60 F-254 plates. Visualization was accomplished by UV irradiation at 254 nm or by staining with ninhydrin (0.3% w/v in glacial acetic acid/n-butyl alcohol 3:97). Flash column chromatography was performed with flash silica gel (particle size 32-63 μm) from Dynamic Adsorbents Inc (Atlanta, Ga.).

Specific rotations of chiral compounds were obtained at the designated concentration and temperature on a Rudolph Research Analytical Autopol H polarimeter using a 0.5 dm cell. Proton and carbon NMR spectra were obtained on Varian 300 and 500 MHz NMR spectrometers (not shown). Chemical shifts are reported as δ values in parts per million (ppm) as referenced to the residual solvents: chloroform (7.27 ppm for ¹H and 77.23 ppm for ¹³C), methanol (3.31 ppm for ¹H and 49.15 ppm for ¹³C), DMSO (2.50 ppm for ¹H and 39.51 ppm for ¹³C), or water (4.80 ppm for ¹H). A minimal amount of 1,4-dioxane was added as the reference standard (67.19 ppm for ¹³C) for carbon NMR spectra in deuterium oxide, and a minimal amount of sodium hydroxide pellet or concentrated hydrochloric acid was added to the NMR sample to aid in the solvation of amino acids which have low solubility in deuterium oxide under neutral conditions. ¹H NMR spectra are tabulated as follows: chemical shift, multiplicity (s=singlet, bs=broad singlet, d=doublet, t=triplet, q=quartet, m=multiplet), number of protons, and coupling constant(s). Mass spectra were obtained at the Laboratory for Biological Mass Spectrometry at the Department of Chemistry, Texas A&M University.

Compounds 1′, 2′, 3′, 4′, 7′ and 8′ were prepared according to literature procedures as previously described. Compound 9′ was purchased from Click Chemistry Tools (Scottsdale, Ariz.). All other reagents were obtained from commercial suppliers and used as received.

### 2. Chemical Synthesis

Compound 5′ was synthesized from fluorescein amine (compound 14′) through carbamate formation (Scheme 5). Compound 6 was synthesized by amide coupling between compound 19′ and 20′ (Scheme 6). Compound 10′ was similarly obtained by coupling between compounds 27′ and 23′ followed by deprotection (Scheme 7).

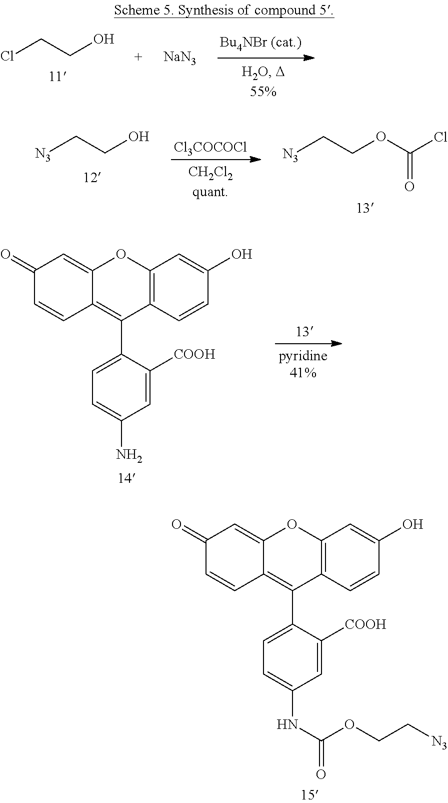

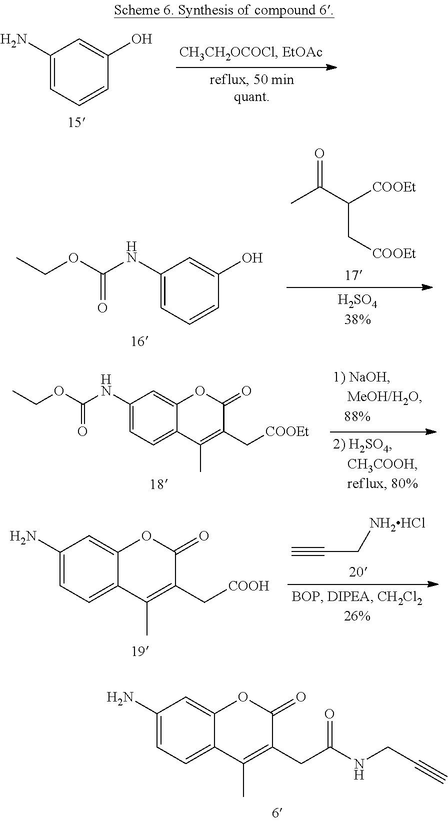

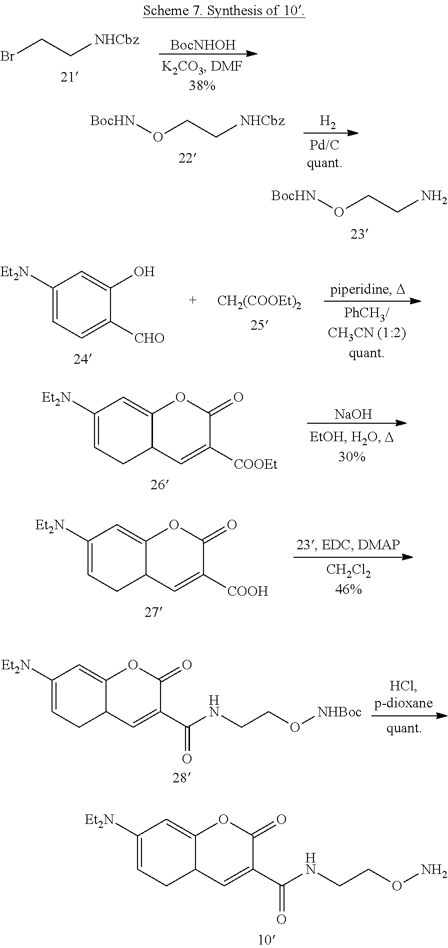

### 2.1—2-Azidoethanol (Compound 12′)

To a solution of 2-chloroethanol (compound 11′, 25.2 g, 0.31 mol) in water (80 mL) was added sodium azide (26.3 g, 0.40 mol) and tetrabutylammonium bromide (2.0 g, 6.2 mmol), and the mixture was stirred at room temperature for 2 h before being heated at 120° C. for 2 h. Sodium chloride was added to the cooled yellow solution until saturation, and the mixture was extracted with ethyl acetate (60 mL×3). The combined organics were dried (MgSO₄, filtered, and evaporated to give a crude yellow oil (31 g). Distillation under an oil pump-generated vacuum (˜0.1 mmHg, by 87-90° C.) afforded compound 12′ (14.9 g, 55%) as a colorless oil. ¹H NMR (CDCl₃, 500 MHz) δ 3.80-3.78 (m, 2H), 3.46 (t, 2H, J=5.0 Hz), 1.88 (bs, 1H); ¹³C NMR (CDCl₃, 125 MHz) δ 61.7, 53.7.

### 2.2—2-Azidoethyl chloroformate (Compound 13′)

To a solution of compound 12′ (82 mg, 0.94 mmol) in anhydrous dichloromethane (0.5 mL) was added trichloromethyl chloroformate (0.13 mmol, 1.1 mmol), and the mixture was stirred at room temperature for 12 h. The volatiles were evaporated to leave crude compound 13′ (0.14 g, quant.) as a yellow oil which was used without further purification. ¹H NMR (CDCl₃, 500 MHz) δ 4.43 (t, 2H, J=5.2 Hz), 3.63 (t, 2H, J=5.0 Hz).

### 2.3—5-((2′-Azidoethyl)oxycarbonylamino)fluorescein (Compound 5′)

To a solution of fluorescein amine isomer I (Aldrich, 0.20 g, 0.58 mmol) in pyridine (2.0 mL, 24.7 mmol) cooled in an ice/water bath was added compound 13′ (0.14 g, 0.94 mmol) dropwise over 5 min with the aid of a small amount of anhydrous dichloromethane (0.1 mL). The mixture was then stirred at room temperature for 48 h, and water (5 mL) was added. The mixture was diluted in ethyl acetate (50 mL), washed with water (10 mL), hydrochloric acid (0.1 N, 10 mL) and brine (10 mL), dried (Na₂SO₄), evaporated, chromatographed (EtOAc/hexanes, 1:1), and crystallized in ethyl acetate/hexanes to give compound 5′ (0.11 g, 41%) as an orange solid. ¹H NMR (DMSO-d₆, 500 MHz) δ 10.32 (s, 1H), 10.12 (s, 2H), 8.12 (s, 1H), 7.78 (dd, 1H, J=8.5, 1.5 Hz), 7.20 (d, 1H, J=8.5 Hz), 6.66 (d, 2H, J=2.0 Hz), 6.59 (d, 2H, J=8.5 Hz), 6.54 (dd, 2H, J=8.7, 2.2 Hz), 4.31 (t, 2H, J=4.7 Hz), 3.65 (t, 2H, J=5.2 Hz); ¹³C NMR (DMSO-d₆, 75 MHz) δ 168.6, 159.5, 153.3, 151.9, 146.2, 140.7, 129.1, 127.1, 125.7, 124.6, 112.6, 112.4, 109.7, 102.2, 83.3, 63.1, 49.7; HRMS (ESI) calculated for C₂₃H₁₇N₄O₇ ([M+H]⁺) 461.1097, found 461.1094.

### 2.4—3-N-(Carbethoxy)aminophenol (Compound 16′)

To a 500 mL round-bottomed flask fitted with a condenser, an addition funnel with pressure-equilibrating side arm, and a magnetic stir bar was charged 3-aminophenol (compound 15′, 50.0 g, 0.45 mol) and ethyl acetate (170 mL), which was dried over MgSO₄ and filtered prior to use. The mixture was heated at reflux to give a grey solution, and ethyl chloroformate (22.2 mL, 0.22 mol) was added dropwise over 50 min. The mixture was cooled to room temperature, filtered, washed with ethyl acetate (100 mL×3) and hexanes (100 mL×3), and dried in vacuo to give the recovered hydrochloride salt of 3-aminophenol (32.9 g, quant.) as an off-white powder. The combined filtrate was evaporated to remove most of the solvents, and hexanes (50 mL) was added. The mixture was frozen at −20° C. for 1 h, crushed, filtered, washed with hexanes (100 mL), and dried in vacuo to give pure 16 (37.0 g) as an off-white solid. The filtrate was again concentrated, mixed with hexanes (50 mL) and frozen, and filtered and treated as above to give more 16′ (4.5 g, quant.) as a brown solid (slightly impure). ¹H NMR (CDCl₃, 500 MHz) δ 7.42 (s, 1H), 7.14 (t, 1H, J=8.0 Hz), 6.96 (bs, 1H), 6.78 (s, 1H), 6.63 (d, 1H, J=8.0 Hz), 6.59 (dd, 1H, J=9.0, 2.5 Hz), 4.24 (q, 2H, J=7.0 Hz), 1.32 (t, 3H, J=7.0 Hz); ¹³C NMR (CDCl₃, 125 MHz) δ 157.2, 154.3, 139.0, 130.1, 110.9, 110.5, 106.1, 61.9, 14.6.

### 2.5—Ethyl 7-carbethoxyamido-4-methylcoumarin-3-acetate (Compound 18′)

To a brown solution of compound 16′ (4.56 g, 25.2 mmol) in 71% sulfuric acid (18 mL) was added diethyl acetylsuccinate (Compound 17′, 5.7 mL, 27.6 mmol) dropwise through an addition funnel over 30 min. After 4 h stirring at room temperature, the mixture was poured into a mixture of ice and water (˜100 g). Upon stirring and trituration the initial white gel solidified, which was filtered and dried to give an off-white solid (6.6 g). The material was suspended in sodium hydroxide (1.0 N, 100 mL) and extracted with dichloromethane (120 mL×3). The combined organics were washed with sodium hydroxide (0.5 N, 50 mL×2) and brine (50 mL), dried (MgSO₄), evaporated, suspended in ether (50 mL), filtered, and dried in vacuo to give compound 18′ (3.2 g, 38%) as a white solid. ¹H NMR (CDCl₃, 500 MHz) δ 7.52 (d, 1H, J=9.0 Hz), 7.41 (d, 1H, J=2.0 Hz), 7.34 (d, 1H, J=8.5 Hz), 7.04 (s, 1H), 4.26 (q, 2H, J=7.2 Hz), 4.20 (q, 2H, J=7.2 Hz), 3.72 (s, 2H), 2.38 (s, 3H), 1.33 (t, 3H, J=7.2 Hz), 1.28 (t, 3H, J=7.0 Hz); ¹³C NMR (CDCl₃, 125 MHz) δ 170.7, 161.9, 153.4, 153.2, 149.0, 141.3, 125.7, 117.8, 115.8, 114.6, 105.8, 61.9, 61.5, 33.2, 15.5, 14.7, 14.4; HRMS (ESI) calculated for C₁₇H₁₉NO₆Li ([M+Li]+) 340.1372, found 340.1373.

### 2.6—7-Carbethoxyamido-4-methylcoumarin-3-acetic acid (Compound 19′)

To a suspension of compound 18′ (1.37 g, 4.1 mmol) in a mixed solvent of methanol (20 mL) and water (20 mL) was added sodium hydroxide (1.0 g, 25.0 mmol), and the mixture was stirred at room temperature for 36 h to give a milky solution. Most of the methanol was evaporated, and the remaining solution was diluted in water (10 mL) and extracted with ethyl acetate (30 mL). The separated aqueous phase was adjusted to pH 1 with concentrated hydrochloric acid, and the precipitate was filtered, washed with water (50 mL) and dichloromethane (50 mL), and dried to give a 7:1 mixture of the corresponding acid of compound 18′ with compound 19′ (1.11 g, 88%) as a pink solid. ¹H NMR (DMSO-d₆, 500 MHz) δ 10.17 (s, 1H, minor product), 10.13 (s, 1H), 7.72 (d, 1H, J=8.0 Hz), 7.54 (s, 1H), 7.39 (d, 1H, J=8.5 Hz), 7.44 (d, 1H, J=8.5 Hz, minor product), 6.57 (d, 1H, J=7.5 Hz, minor product), 6.41 (s, 1H, minor product), 6.05 (s, 2H, minor product), 4.16 (q, 2H, J=6.8 Hz), 3.70 (s, 2H, minor product), 3.56 (s, 2H), 2.34 (s, 3H), 2.25 (s, 3H, minor product), 1.25 (t, 3H, J=7.0 Hz); ¹³C NMR (DMSO-d₆, 125 MHz) δ 171.7, 161.0, 153.5, 152.6, 149.0, 142.4, 126.3, 117.4, 114.6, 114.5, 104.3, 60.8, 32.8, 15.0, 14.5.

The above crude mixture of products (1.11 g, 3.6 mmol) was dissolved in concentrated sulfuric acid (1.5 mL) and acetic acid (1.5 mL) in a 100 mL flask and heated at reflux for 3 h to give a dark solution. Upon cooling down to room temperature ice/water (15 g) was added followed by another 25 mL of water. Charcoal (0.5 g) and Celite 545 (0.5 g) were then added, and the mixture was heated at reflux for 10 min. The hot mixture was carefully filtered to give a red filtrate, which was evaporated to a volume of −15 mL. The solution was allowed to stand at room temperature overnight, and further cooling at −20° C. did not yield more precipitate. The mixture was filtered, washed with cold water (4 mL) and cold ethanol (10 mL), and dried to give 19 (0.71 g, 80%) as a brown solid. ¹H NMR (DMSO-d₆, 500 MHz) δ 7.50 (d, 1H, J=9.0 Hz), 6.65 (dd, 1H, J=8.7, 1.8 Hz), 6.51 (d, 1H, J=2.0 Hz), 3.50 (s, 2H), 2.27 (s, 3H); ¹³C NMR (DMSO-d₆, 125 MHz) δ 172.0, 161.5, 153.9, 150.8, 149.7, 126.6, 113.6, 112.3, 110.2, 99.8, 32.6, 14.9.

### 2.7—2-(7-Amino-4-methyl-2-oxo-2H-chromen-3-yl)-N-(prop-2-yn-1-yl)acetamide (Compound 6′)

To a solution of compound 19′ (10 mg, 43 μmol), (benzotriazol-1-yloxy)tris(dimethylamino)phosphonium hexafluorophosphate (BOP, 26 mg, 62 μmol, N,N-diisopropylethylamine (20 μL, 0.12 mmol) in anhydrous DMF (0.2 mL) was added propargylamine hydrochloride (compound 20′, 7.5 mg, 78 mop, and the mixture was stirred at room temperature for 14 h. The mixture was diluted in ethyl acetate (40 mL), washed with sodium hydroxide (0.5 N, 10 mL), hydrochloric acid (0.5 N, 10 mL) and brine (10 mL), dried (Na₂SO₄), evaporated, and chromatographed (EtOAc/hexanes, 2:1 followed by 5% MeOH in CH₂Cl₂) to give compound 6′ (3.0 mg, 26%) as a yellow solid. ¹H NMR (DMSO-d₆, 500 MHz) δ 8.31 (t, 1H, J=5.7 Hz), 7.45 (d, 1H, J=8.5 Hz), 6.57 (dd, 1H, J=8.7, 2.2 Hz), 6.40 (d, 1H, J=2.0 Hz), 6.04 (s, 2H), 3.83 (dd, 2H, J=5.5, 2.5 Hz), 3.40 (s, 2H), 3.09 (s, 2H), 2.24 (s, 3H); ¹³C NMR (DMSO-d₆, 75 MHz) δ 169.1, 161.6, 154.1, 152.4, 150.0, 126.3, 113.1, 111.3, 109.4, 99.4, 81.3, 72.8, 33.5, 28.0, 14.9; HRMS (ESI) calculated for C₁₅H₁₄N₂O₃Na ([M+Na]⁺) 293.0902, found 293.0914.

### 2.8—N-Boc-O-(2-(benzyloxycarbonylamino)ethyl)hydroxylamine (Compound 22′)

To a solution of compound 21′ (0.30 g, 1.2 mmol) and N-Boc-hydroxylamine (0.16 g, 1.2 mmol) in anhydrous DMF (1.5 mL) was added potassium carbonate (0.44 g, 3.2 mmol), and mixture was stirred at room temperature for 24 h. Water (10 mL) was then added, and the mixture was extracted with ether (50 mL). The organic phase was washed with sodium hydroxide (0.5 N, 10 mL), hydrochloric acid (0.5 N, 10 mL) and brine (10 mL), dried (Na₂SO₄), evaporated, and chromatographed (EtOAc/hexanes, 1:5) to give 22′ (0.14 g, 38%) as a colorless oil. ¹H NMR (CDCl₃, 500 MHz) δ 7.47 (bs, 1H), 0.38-7.29 (m, 5H), 5.80 (bs, 1H), 5.12 (s, 2H), 3.86 (t, 2H, J=4.5 Hz), 3.44-3.43 (m, 2H), 1.47 (s, 9H); ¹³C NMR (CDCl₃, 125 MHz) δ 158.0, 157.4, 137.2, 129.1, 128.9, 128.6, 82.7, 76.2, 67.3, 39.7, 28.8; HRMS (ESI) calculated for C₁₅H₂₂N₂O₅Na ([M+Na]⁺) 333.1426, found 333.1418.

### 2.9—tert-Butyl 2-aminoethoxycarbamate (Compound 23′)

To a solution of compound 22′ (0.33 g, 1.1 mmol) in methanol (20 mL) was added palladium on activated carbon (10% Pd, 0.11 g, 0.1 mmol), and the mixture was hydrogenated under a H₂ balloon at room temperature for 5 h. The mixture was filtered over Celite and evaporated to give 23′ (0.20 g, quant.) as a grey oil. ¹H NMR (CDCl₃, 500 MHz) δ 3.93 (t, 2H, J=5.0 Hz), 2.99 (t, 2H, J=5.2 Hz), 1.48 (s, 9H), 1.46 (s, 2H). The material was used without further purification.

### 2.10—Ethyl 7-(diethylamino)coumarin-3-carboxylate (Compound 26′)

To a solution of 4-(diethylamino)salicylaldehyde (compound 24′, 5.93 g, 30.1 mmol) and diethyl malonate (7.6 g, 47.0 mmol) in a mixed solvent of toluene and acetonitrile (1:2, 210 mL) was added piperidine. (8.9 mL, 90.1 mmol), and the red solution was heated at reflux for 10 h. The solvent was evaporated under reduced pressure, and the residue was directly chromatographed (EtOAc/hexanes, 1:3) to give 26′ (9.2 g, quant.) as a yellow oil. ¹H NMR (CDCl₃, 500 MHz) δ 8.34 (s, 1H), 7.29 (d, 1H, J=8.5 Hz), 6.54 (dd, 1H, J=8.7, 2.2 Hz), 6.35 (d, 1H, J=2.5 Hz), 4.29 (q, 2H, J=7.2 Hz), 3.37 (q, 2H, J=7.2 Hz), 1.32 (t, 3H, J=7.2 Hz), 1.16 (t, 6H, J=7.2 Hz); ¹³C NMR (CDCl₃, 75 MHz) δ 164.2, 158.4, 152.9, 149.2, 131.1, 109.6, 108.8, 107.6, 96.6, 61.1, 45.1, 14.4, 12.5.

### 2.11—7-(Diethylamino)coumarin-3-carboxylic acid (Compound 27′)

To a solution of compound 26′ (3.34 g, 11.5 mmol) in ethanol (30 mL) was added sodium hydroxide (1.0 N, 20.0 mL, 20.0 mmol), and a yellow precipitate quickly formed. The mixture was heated at reflux for 3 h to give a clear red solution, which was cooled to room temperature and filtered. The filtrate was adjusted to pH 3 with hydrochloric acid (2 N, ˜18 mL), filtered, washed with water (30 mL), ethanol (10 mL) and ether (30 mL), and dried to give compound 27′ (0.90 g, 30%) as an orange solid. ¹H NMR (CD₃OD, 500 MHz) δ 8.62 (s, 1H), 7.58 (d, 1H, J=9.0 Hz), 6.86 (dd, 1H, J=9.0, 2.5 Hz), 6.62 (d, 1H, J=2.5 Hz), 3.56 (q, 2H, J=7.0 Hz), 1.25 (t, 6H, J=7.2 Hz); HRMS (ESI) calculated for C₁₄H₁₆NO₄ ([M+H]⁺) 262.1079, found 262.1073.

### 2.12—tert-Butyl 2-(7-(diethylamino)-2-oxo-4-a,5-dihydro-2H-chromene-3-carboxamido)ethoxycarbamate (Compound 28′)

To a solution of compound 27 (0.146 g, 0.56 mmol), 4-(dimethylamino)pyridine (44 mg, 0.36 mmol) and compound 23′ (74 mg, 0.42 mmol) in anhydrous dichloromethane (2 mL) was added N-(3-dimethylaminopropyl)-N′-ethylcarbodiimide hydrochloride (EDC hydrochloride, 0.136 g, 0.71 mmol), and the mixture was stirred at room temperature for 12 h. The mixture was diluted in ethyl acetate (50 mL), washed with sodium hydroxide (0.5 N, 10 mL), hydrochloric acid (0.5 N, 10 mL) and brine (10 mL), dried (Na₂SO₄), evaporated, and chromatographed (EtOAc/hexanes, 1:3 to 1:1) to give compound 28′ (81 mg, 46%) as a yellow oil which solidified upon standing. ¹H NMR (CDCl₃, 300 MHz) δ 9.06 (t, 1H, J=6.0 Hz), 8.67 (s, 1H), 8.07 (s, 1H), 7.40 (d, 1H, J=9.0 Hz), 6.63 (dd, 1H, J=8.8, 2.5 Hz), 6.48 (d, 1H, J=2.4 Hz), 3.96 (t, 2H, J=5.1 Hz), 3.70 (dt, 2H, J=5.4, 5.4 Hz), 3.44 (q, 4H, J=7.2 Hz), 1.47 (s, 9H), 1.22 (t, 6H, J=7.0 Hz); ¹³C NMR (CDCl₃, 75 MHz) δ 164.2, 162.8, 157.8, 156.9, 152.6, 148.4, 131.3, 110.2, 108.6, 96.9, 81.5, 74.9, 45.3, 37.8, 28.4, 12.5; HRMS (ESI) calculated for C₂₁H₃₀N₃O₆ ([M+H]⁺) 420.2135, found 420.2129; calculated for C₂₁H₂₉N₃O₆Li ([M+Li]⁺) 426.2216, found 426.2214; calculated for C₂₁H₂₉N₃O₆Na ([M+Na]⁺) 442.1954, found 442.2010.

### 2.13—N-(2-(Aminooxy)ethyl)-7-(diethylamino)-2-oxo-4-a,5-dihydro-2H-chromene-3-carboxamide

(Compound 10′). To a solution of compound 28′ (62 mg, 0.15 mmol) in 1,4-dioxane (1.0 mL) was added hydrogen chloride in dioxane (4.0 M, 0.3 mL, 1.2 mmol), and the mixture was stirred at room temperature for 4 h. Water (10 mL) was added followed by saturated sodium bicarbonate (20 mL), and the mixture was extracted with chloroform (50 mL). The separated organic phase was washed with brine (10 mL), dried (Na₂SO₄), and evaporated to afford compound 10′ (48 mg, quant.) as a yellow oil. ¹H NMR (CDCl₃, 500 MHz) δ 9.03 (s, 1H), 8.69 (s, 1H), 7.42 (d, 1H, J=8.5 Hz), 6.63 (dd, 1H, J=9.2, 1.7 Hz), 6.48 (d, 1H, J=2.0 Hz), 3.88 (bs, 2H), 3.69 (bs, 2H), 3.44 (q, 4H, J=7.2 Hz), 1.23 (t, 6H, J=7.0 Hz); ¹³C NMR (CDCl₃, 125 MHz) δ 163.9, 162.9, 157.8, 152.7, 148.4, 131.3, 110.2, 110.1, 108.5, 96.7, 45.2, 38.5, 12.6; HRMS (ESI) calculated for C₁₆H₂₂N₃O₆ ([M+H]⁺) 320.1610, found 320.1616.

### 3. Primer and Gene Sequences

### 3.1—Primer Sequences

Forward primer NP2:
(SEQ ID NO: 8)
agcgcggccgcgtcgacggtaccctcgagtctggtaaag.
Reverse primer NP2:
(SEQ ID NO: 9)
attgcggccgcccatggtatatctccttcttatacttaac.
F1-QBP3TAG:
(SEQ ID NO: 10)
tatacatatggcctaggattaaaaattagttgtcgc.
R1-QBP141TAA:
(SEQ ID NO: 11)
cagttccatataggcttaatcgatgttcgggaa.
F2-QBP141TAA:
(SEQ ID NO: 12)
ttcccgaagaatcgattaagcctatatggaactg.
R2-QBP:
(SEQ ID NO: 13)
gagggtacctcagtgatggtgatggtgatgtttcggttcagtacc.
PyIRS->AcKRS:
(SEQ ID NO: 14)
acctgcgataccggtttccacccaag.
PyIRS->AcKRS R:
(SEQ ID NO: 15)
cttaagttacaggttggtagaaatccc.

### 3.2—Genes Sequences

QBP2m (the underlined sequence represents the amber mutation and ochre mutation in the sequences encoding amino acid positions 3 and 141 of the polypeptide, respectively) (SEQ ID NO:16):

Atggcctaggataaaaaattagttgtcgcgacggataccgccttcgttccgtttgaatttaaacagggcgataaatatgt
gggctttgacgttgatctgtgggctgccatcgctaaagagctgaagctggattacgaactgaagccgatggatttcagtgggatc
attccggcactgcaaaccaaaaacgtcgatctggcgctggcgggcattaccatcaccgacgagcgtaaaaaagcgatcgatttc
tctgacggctactacaaaagcggcctgttagtgatggtgaaagctaacaataacgatgtgaaaagcgtgaaagatctcgacggg
aaagtggttgcggtgaagagcggcactggctccgttgattacgcgaaagcaaacatcaaaactaaagatctgcgtcagttcccg
aacatcgattaagcctatatggaactgggcaccaaccgcgcagacgccgttctgcacgatacgccaaacattctgtacttcatca
aaaccgccggtaacggtcagttcaaagcggtaggcgactctctggaagcgcagcaatacggtattgcgttcccgaaaggtagc
gacgagctgcgtgacaaagtcaacggcgcgttgaaaaccctgcgcgagaacggaacttacaacgaaatctacaaaaaatggtt
cggtactgaaccgaaacatcaccatcaccatcactga
MmAcKRS (SEQ ID NO: 17):
atggataaaaaaccactaaacactctgatatctgcaaccgggctctggatgtccaggaccggaacaattcataaaata
aaacaccacgaagtctctcgaagcaaaatctatattgaaatggcatgcggagaccaccttgttgtaaacaactccaggagcagca
ggactgcaagagcgctcaggcaccacaaatacaggaagacctgcaaacgctgcagggtttcggatgaggatctcaataagttc
ctcacaaaggcaaacgaagaccagacaagcgtaaaagtcaaggtcgtttctgcccctaccagaacgaaaaaggcaatgccaa
aatccgttgcgagagccccgaaacctcttgagaatacagaagcggcacaggctcaaccttctggatctaaattttcacctgcgata
ccggtttccacccaagagtcagtttctgtcccggcatctgtttcaacatcaatatcaagcatttctacaggagcaactgcatccgca
ctggtaaaagggaatacgaatcccattacatccatgtctgcccctgttcaggcaagtgcccccgcacttacgaagagccagactg
acaggcttgaagtcctgttaaacccaaaagatgagatttccctgaattccggcaagcctttcagggagcttgagtccgaattgctct
ctcgcagaaaaaaagacctgcagcagatctacgcggaagaaagggagaattatctggggaaactcgagcgtgaaattaccag
gttctttgtggacaggggttttctggaaataaaatccccgatcctgatccctcttgagtatatcgaaaggatgggcattgataatgat
accgaactttcaaaacagatcttcagggttgacaagaacttctgcctgagacccatgatggctccaaacctgctgaactacgccc
gcaagcttgacagggccctgcctgatccaataaaaatttttgaaataggcccatgctacagaaaagagtccgacggcaaagaac
acctcgaagagtttaccatgctgaacttctgccagatgggatcgggatgtacacgggaaaatcttgaaagcataattacggacttc
ctgaaccacctgggaattgattttcaagatcgtaggcgattccttcatggtctatggggatacccttgatgtaatgcacggagacctg
gaactttcctctgcagtagtcggacccataccgcctgaccgggaatggggtattgataaaccctggataggggcaggtttcgggc
tcgaacgccttctaaaggttaaacacgactttaaaaatatcaagagagctgcaaggtccgagcttactataacgggatttctacca
acctgtaa
MmPyIRS (SEQ ID NO: 18):
atggataaaaaaccactaaacactctgatatctgcaaccgggctctggatgtccaggaccggaacaattcataaaata
aaacaccacgaagtctctcgaagcaaaatctatattgaaatggcatgcggagaccaccttgttgtaaacaactccaggagcagca
ggactgcaagagcgctcaggcaccacaaatacaggaagacctgcaaacgctgcagggtttcggatgaggatctcaataagttc
ctcacaaaggcaaacgaagaccagacaagcgtaaaagtcaaggtcgtttctgcccctaccagaacgaaaaaggcaatgccaa
aatccgttgcgagagccccgaaacctcttgagaatacagaagcggcacaggctcaaccttccggatctaaattttcacctgcgata
ccggtttccacccaagagtcagtttctgtcccggcatctgtttcaacatcaatatcaagcatttctacaggagcaactgcatccgca
ctggtaaaagggaatacgaaccccattacatccatgtctgcccctgttcaggcaagtgcccccgcacttacgaagagccagact
gacaggcttgaagtcctgttaaacccaaaagatgagatttccctgaattccggcaagcctttcagggagcttgagtccgaattgct
ctctcgcagaaaaaaagacctgcagcagatctacgcggaagaaagggagaattatctggggaaactcgagcgtgaaattacca
ggttctttgtggacaggggttttctggaaataaaatccccgatcctgatccctcttgagtatatcgaaaggatgggcattgataatga
taccgaactttcaaaacagatcttcagggttgacaagaacttctgcctgagacccatgcttgctccaaacctttacaactacctgcg
caagcttgacagggccctgcctgatccaataaaaatttttgaaataggcccatgctacagaaaagagtccgacggcaaagaaca
cctcgaagagtttaccatgctgaacttctgccagatgggatcgggatgcacacgggaaaatcttgaaagcataattacggacttcc
tgaaccacctgggaattgatttcaagatcgtaggcgattcctgcatggtctatggggatacccttgatgtaatgcacggagacctg
gaactttcctctgcagtagtcggacccataccgcttgaccgggaatggggtattgataaaccctggataggggcaggtttcgggc
ccgaacgccttctaaaggttaaacacgactttaaaaatatcaagagagctgcaaggtccgagtcttactataacgggatttctacca
acctgtaa

### 4. Construction of Plasmids

All the plasmid structures were confirmed by DNA sequencing. All oligonucleotide primers were purchased from Integrated DNA Technologies, Inc. All the PCR reactions were performed with Phusion® High-Fidelity DNA Polymerase from New England Biolabs Inc. (Ipswich, Mass.). All the restriction enzymes, T4 DNA ligase and T4 polynucleotide kinase (T4 PNK) were purchased from New England Biolabs Inc. (Ipswich, Mass.).

### 4.1—Construction of pETtrio-PylT-PylRS-MCS

The plasmid pPylRS-Pyl-MCS was derived from pPylRS-pylT-GFPITAG149TAA. Forward primer NP2 and reverse primer NP2 were used to clone the whole plasmid without the gene GFP1TAG149TAA. Meanwhile, four restriction sites, Nco I, Not I, Sal I and Kpn I were introduced to the positions where GFPITAG149TAA was located in the original plasmid.

### 4.2—Construction of pETtrio-pylT-PylRS-QBP3TAG141TAA

F1-QBP3TAG and R1-QBP141TAA were used to clone the first part of site-mutated glutamine binding protein (QBP) from E. coli TOP 10 cell. The second part of QBP3TAG141TAA gene was cloned in the same manner by using F2-QBP141TAA and R2—QBP as the two primers. Overlap PCR was performed with F1-QBP3TAG and R2-QBP as the two primers and the two fragments obtained from the PCR reactions mentioned above to afford QBP3TAG141TAA, which was inserted to pETtrio-PylT-PylRS-MCS with Nco I at 5′ end and Kpn I at 3′ end.

### 4.3—Construction of pETtrio-pylT-MmAcKRS-pylT-QBP3TAG141TAA

pKTS-MmAcKRS, the plasmid contains MmAcKRS that takes compound 4 and efficiently evolved from MmPylRS. To construct pETtrio-pylT-MmAcKRS-pylT-QBP3TAG141TAA, pETtrio-pylT-PylRS-QBP3TAG141TAA was used. Because MmAcKRS was derived from PylRS and all the mutations are beyond the 600 base pairs in the gene, PylRS->AcKRS F, a forward primer basing on the sequence of the PylRS at 450 bp was designed. Together with PylRS->AcKRS R, PylRS was converted to MmAcKRS with Age I at the 5′ end and Afl II at the 3′ end.

### 5. Protein Expression and Purification

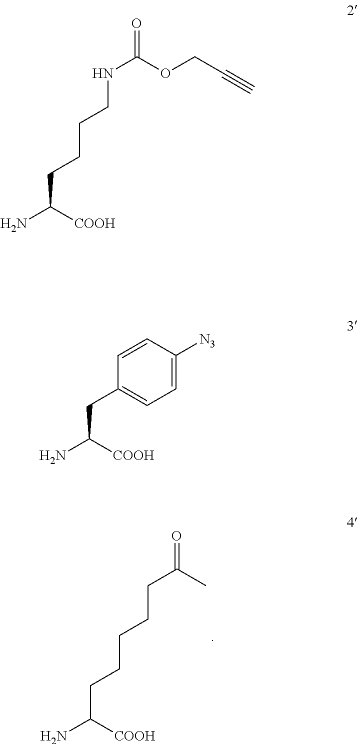 [SMILES: C#CCOC(=O)NCCCC[C@H](N)C(=O)O ; [N-]=[N+]=Nc1ccc(C[C@H](N)C(=O)O)cc1 ; CC(=O)CCCCCC(N)C(=O)O]

### 5.1—Expression of QBP(3′+2′)

E. coli BL21 cells co-transformed with pETtrio-pylT-MmAcKRS-pylT-QBP3TAG141TAA and pEVOL-AzFRS were grown in 2TY medium (150 mL) with 100 μg/mL ampicillin and 34 μg/mL chloramphenicol overnight. The culture was inoculated into 2YT medium (450 mL) with the same concentration of antibiotics. IPTG (500 mM), arabinose (0.2% w/v), together with NAA #2′ and NAA #3′ (both 1 mM) were added into the cell culture after the OD₆₀₀ reached 1.2-1.4. The cell culture was incubated at 37° C. for 8 h, and the cells were harvested by centrifugation at 4000 r.p.m. for 20 min at 4° C. and re-suspended in 50 mL of lysis buffer (50 mM HEPES, 500 mM NaCl, 10 mM imidazole, pH 7.8). The re-suspended cells were sonicated in an ice/water bath four times (4 min each, 10 min interval to cool the suspension below 10° C. before the next run) and the lysate was clarified by centrifugation at 10000 r.p.m. for 40 min at 4° C. The supernatant was then incubated with 3 mL of Ni Sepharose™ 6 Fast Flow from GE Healthcare (Little Chalfont, United Kingdom) for 1 h, and then washed with 100 mL of lysis buffer. QBP(3′+2′) was then eluted out with 12 mL of elution buffer (50 mM HEPES, 500 mM NaCl, 250 mM imidazole, pH 7.8) and concentrated by Amicon Ultra-15 Centrifugal Filter Units—10,000 NMWL from Millipore (Billerica, Mass.) to 3 mL. The buffer was then changed to ammonium bicarbonate (ABC, 20 mM, pH 8.1) by dialysis. The concentration was determined by BCA protein assay kit from Thermo Fisher Scientific Inc. (Rockford, Ill.). According to the concentration, QBP(3′+2′) expression yield was 12 mg/L from the 2YT medium.

### 5.2—Expression of QBP(3′+4′)

E. coli BL21 cells were co-transformed with pETtrio-pylT-MmAcKRS-pylTQBP3TAG141TAA and pEVOL-AzFRS for the expression of QBP(3′+4′), which followed the same procedure of the expression of QBP(3′+2′) except NAA #4′ (2 mM) was supplemented with NAA #2′ (1 mM) instead of NAA #3′. Purified QBP(3′+4′) was dialyzed against phosphate buffered saline (pH 6.4) for the following labeling reactions. The expression yield for QBP(3′+4′) was 11 mg/L from the 2YT medium.

### 6. Protein Labeling Procedures

### 6.1—Copper-Catalyzed Azide-Alkyne Cycloaddition (CuAAC)

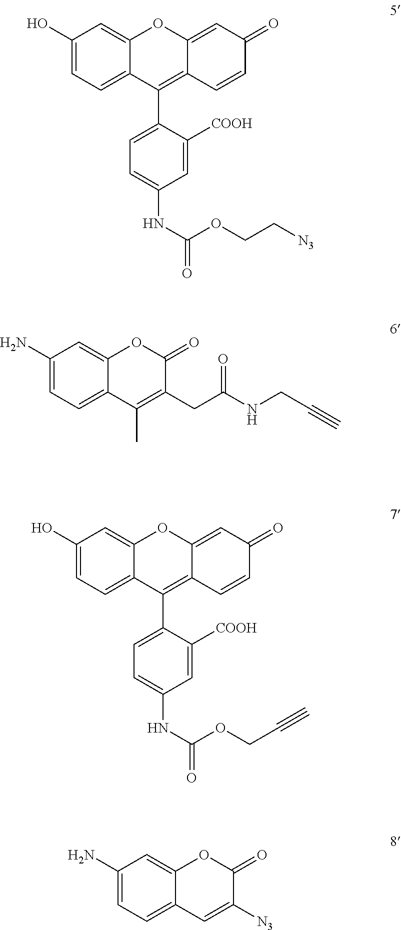 [SMILES: [N-]=[N+]=NCCOC(=O)Nc1ccc(-c2c3ccc(=O)cc-3oc3cc(O)ccc23)c(C(=O)O)c1 ; C#CCNC(=O)Cc1c(C)c2ccc(N)cc2oc1=O ; C#CCOC(=O)Nc1ccc(-c2c3ccc(=O)cc-3oc3cc(O)ccc23)c(C(=O)O)c1 ; [N-]=[N+]=Nc1cc2ccc(N)cc2oc1=O]

To QBP(3′+2′) (concentration varied from 0.017 mM to 0.072 mM in 20 mM ABC buffer, 270 μL, pH 8.1) was added CuSO₄ (100 μM), NiCl₂ (1 mM), tris[(1-benzyl-1H-1,2,3-triazol-4-yl)methyl]amine (TBTA, stock solution in DMSO, 500 μM) and one of the dyes (5, 6, 7 and 8, stock solutions in DMSO, 50 equiv. to the protein) sequentially, followed by sodium ascorbate (5 mM). The reaction was performed at room temperature for 3 h. Then ethylenediaminetetraacetic acid (EDTA, pH 8.0, 5 μL, 0.5 M final concentration) was added to the reaction mixture to chelate the two metals. The reaction product was transferred into lysis buffer (50 mM HEPES, 500 mM NaCl, 10 mM imidazole, pH 7.8, 10 mL) with Ni Sepharose™ 6 Fast Flow (1 mL) and incubated at 4° C. for 1 h. The resin was loaded onto an empty column and the catalysts were washed away by lysis buffer (100 mL). The labeled QBP(3′+2′) was eluted out by elution buffer (50 mM HEPES, 500 mM NaCl, 250 mM imidazole, pH 7.8, 6 mL), concentrated, dialyzed against ABC buffer (20 mM, pH 8.1) and then analyzed by mass spectrometry. The second CuAAC labeling was performed in the same manner with the appropriate dye to afford doubly labeled QBP(3′+2′).

### 6.2—Catalyst-Free Labeling

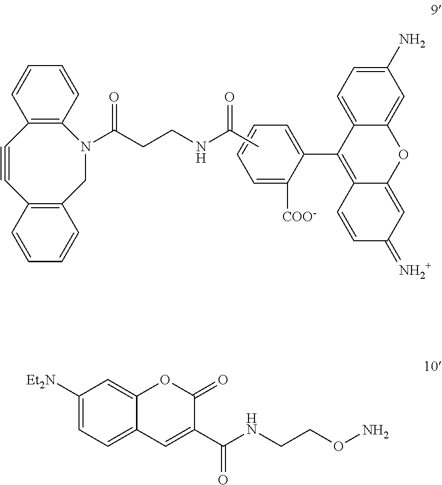

To QBP(3′+4′) (0.024 mM to 0.035 mM) was added compound 9′ (50 equiv.) and compound 10′ (10 equiv.). The reaction was performed at room temperature overnight. The reaction product was transferred into lysis buffer (50 mM HEPES, 500 mM NaCl, 10 mM imidazole, pH 7.8, 10 mL) with Ni Sepharose™ 6 Fast Flow (1 mL) and incubated at 4° C. for 1 h. The resin was loaded onto an empty column and the dye was washed away by lysis buffer (400 mL). The labeled QBP(3′+4′) was eluted out by elution buffer (50 mM HEPES, 500 mM NaCl, 250 mM imidazole, pH 7.8, 6 mL), concentrated, dialyzed against ABC buffer (20 mM, pH 8.1) and then analyzed by mass spectrometry. It is noteworthy that excessive lysis buffer must be used to completely remove compound 9′, which has very poor solubility in water.

### 7. Mass Spectrometry Analysis

Nanoelectrospray ionization in positive mode was performed using an Applied Biosystems QSTAR Pulsar (Concord, ON, Canada) equipped with a nanoelectrospray ion source. Solution was flowed at 700 mL/min through a 50 μM ID fused-silica capillary that was tapered at the tip. Electrospray needle voltage was held at 2100 V.

### 8. FRET Assay

QBP(3′+2′)-6′-5′ and QBP(3′+4′)-9′-10′ were diluted with various concentrations of guanidine hydrochloride (GndCl, 0 M, 1 M, 2 M, 3 M, 4 M, 5 M and 6 M, respectively) in PBS buffer (pH 7.8). The fluorescent emission of those solutions was tested by QuantaMaster™ 40 Intensity Based Spectrofluorometer from Photon Technology International Inc. (Birmingham, N.J.) with excitation at 350 nm (QBP(3′+2′)) or 430 nm (QBP(3′+4′)). Emission change based on the concentration of GndCl was plotted. All measurements were taken on freshly made samples and the data was collected every 0.2 second with 0.5 nm intervals.

While illustrative embodiments have been illustrated and described, it will be appreciated that various changes can be made therein without departing from the spirit and scope of the invention.

The embodiments of the invention in which an exclusive property or privilege is claimed are defined as follows:

1. A method of incorporating at least two different noncanonical amino acids into a target protein, comprising:
(a) providing to a translation system a first polynucleotide encoding a first tRNA mutated to suppress an ochre codon, an opal codon, or a four-base codon, or the first mutant tRNA encoded thereby;
(b) providing to the translation system a second polynucleotide encoding a first aminoacyl tRNA synthetase (aaRS) that may be a mutant or wild-type, or the first aaRS encoded thereby, wherein the first aaRS is capable of charging the first mutant tRNA with a first noncanonical amino acid (NAA);
(c) providing to the translation system a third polynucleotide encoding a second tRNA mutated to suppress an amber codon, or the second mutant tRNA encoded thereby, wherein the second mutant tRNA is orthogonal to the first mutant tRNA;
(d) providing to the translation system a fourth polynucleotide encoding a second aaRS that may be a mutant or wild-type, or the second aaRS encoded thereby, wherein the second aaRS is capable of charging the second mutant tRNA with a second NAA that is different than the first NAA and wherein the second aaRS is orthogonal to the first aaRS;
(e) providing to the translation system the first NAA;
(f) providing to the translation system the second NAA;
(g) providing to the translation system a fifth polynucleotide encoding the target protein, wherein the fifth polynucleotide comprises (1) a sequence encoding an ochre codon, an opal codon, or a four-base codon at a first specified position, and (2) a sequence encoding an amber codon at a second specified position; and
(h) allowing translation of the fifth polynucleotide, thereby incorporating into the target protein (1) the first NAA at the first specified position and (2) the second NAA at the second specified position.

2. The method of claim 1, wherein the provision of steps (a)-(g) occurs in a host cell.

3. The method of claim 2, wherein the host cell is a bacteria cell, a yeast cell, an insect cell, or a mammalian cell.

4. The method of claim 2, wherein the host cell is an Escherichia coli cell, a Bacillus subtilis cell, a Saccharomyces cerevisiae cell, a Pichia pastoris cell, an SF9 cell, a Chinese Hamster Ovary (CHO) cell, or a human cell.

5. The method of claim 2, wherein the first and second mutant tRNAs are orthogonal to the endogenous tRNAs in the host cell.

6. The method of claim 2, wherein the first and second mutant aaRSs are orthogonal to the endogenous aaRSs in the host cell.

7. The method of claim 1, wherein the provision in steps (a)-(g) occurs in a cell-free system.

8. The method of claim 7, wherein the cell-free environment comprises a cell lysate.

9. The method of claim 1, wherein the first NAA and the second NAA are capable of forming a reactive pair.

10. The method of claim 9, wherein one of the first NAA and second NAA comprises a donor moiety and the other NAA comprises an acceptor moiety, wherein the donor and acceptor moieties are capable of undergoing Förster resonance energy transfer (FRET).

11. The method of claim 10, wherein one or both of the donor and acceptor moieties is incorporated into the cognate NAA before the cognate NAA has been incorporated into the target protein.

12. The method of claim 10, wherein one or both of the donor and acceptor moieties is incorporated into the cognate NAA after the cognate NAA has been incorporated into the target protein.

13. The method of claim 1, wherein the first aaRS is derived from a first organism and the second aaRS is derived from a second organism.

14. The method of claim 1, wherein the first aaRS is derived from a Methanococcus mazei aaRS or a Methanococcus barkeri aaRS.

15. The method of claim 1, wherein the first aaRS is pyrrolysyl-tRNA synthetase (PylRS) or a mutant PylRS.

16. The method of claim 1, wherein the second aaRS is derived from a Methanococcus jannascii aaRS.

17. The method of claim 1, wherein the second aaRS is a mutant M. jannaschii tyrosyl-tRNA synthetase (MjTyrRS).

18. A translation system comprising:
(a) a first polynucleotide encoding a first tRNA mutated to suppress an ochre codon, an opal codon, or a four-base codon, or the first mutant tRNA encoded thereby;
(b) a second polynucleotide encoding a first aminoacyl tRNA synthetase (aaRS) that may be a mutant or wild-type, or the first aaRS encoded thereby, wherein the first aaRS is capable of charging the first mutant tRNA with a first noncanonical amino acid (NAA);
(c) a third polynucleotide encoding a second tRNA mutated to suppress an amber codon, or the second tRNA encoded thereby, wherein the second mutant tRNA is orthogonal to the first mutant tRNA;
(d) a fourth polynucleotide encoding second aaRS that may be a mutant or wild-type, or the second aaRS encoded thereby, wherein the second aaRS is capable of charging the second mutant tRNA with a second NAA that is different than the first NAA and wherein the second aaRS is orthogonal to the first aaRS; and
(e) a fifth polynucleotide encoding a target protein, wherein the fifth polynucleotide comprises (1) a sequence encoding an ochre codon, an opal codon, or a four-base codon at a first specified position, and (2) a sequence encoding an amber codon at a second specified position.

19. The translation system of claim 18, wherein a host cell comprises (a)-(e).

20. The translation system of claim 18, wherein (a)-(e) are in a cell-free translation system.

21. The translation system of claim 18, further comprising the first NAA, the second NAA, or both.

22. A mutant tRNA^{Pyl} (pylT) comprising a mutation to suppress an ochre codon, an opal codon, or a four-base codon.

23. The mutant pylT of claim 22, wherein the mutant pylT is pylT_{UUA}, pylT_{UCA}, or pylT_{UCUA}.

24. The mutant pylT of claim 22, is aminoacylated with an NAA.
